(19) United States
(12) Patent Application Publication
(10) Pub. No.: US 2026/0015674 A1
(43) Pub. Date: Jan. 15, 2026

(54) USE OF PA-TN5 TO GENERATE DNA LIBRARIES

(71) Applicant: The Trustees of Columbia University in the City of New York, New York, NY (US)

(72) Inventors: Zhiguo Zhang, Fort Lee, NJ (US); Regina Santella, Teaneck, NJ (US); Hui Zhou, New York, NY (US); Hui-Chen Wu, New York, NY (US)

(21) Appl. No.: 19/227,903

(22) Filed: Jun. 4, 2025

Related U.S. Application Data
(63) Continuation of application No. PCT/US2023/085339, filed on Dec. 21, 2023.
(60) Provisional application No. 63/476,517, filed on Dec. 21, 2022.

(51) Int. Cl. C12Q 1/6886; C12N 15/10

(52) U.S. Cl. CPC C12Q 1/6886; C12N 15/1065; C12Q 2600/154

(57) ABSTRACT

Disclosed herein are methods of preparing a DNA library using a pA-Tn5 transposase, methods of identifying a modification of interest in DNA using a pA-Tn5 transposase, and kits for carrying out the disclosed methods.

### CROSS-REFERENCE TO RELATED APPLICATIONS

This application is a continuation of International Patent Application No. PCT/US2023/085339, filed Dec. 21, 2023, which claims priority to U.S. Provisional Application Ser. No. 63/476,517, filed on Dec. 21, 2022, the disclosures of each of which are hereby incorporated by reference in their entireties.

### GOVERNMENT RIGHTS

This invention was made with government support under R35 GM118015 awarded by the National Institutes of Health. The government has certain rights in the invention.

### TECHNICAL FIELD

Disclosed herein are methods of preparing DNA libraries.

### BACKGROUND

The generation of DNA libraries from genomic DNA as well as analysis of DNA methylation by methylated DNA immunoprecipitation often require the use of sonication to shear genomic DNA into small fragments. However, sonication leads to a loss of DNA. Furthermore, extensive and inefficient procedures need to be followed for library preparation. Therefore, large amount of input genomic DNA are needed for most current procedures.

### SUMMARY

Disclosed herein are methods of preparing a DNA library from isolated DNA, the methods comprising incubating the isolated DNA with a fusion protein comprising protein A and a Tn5 transposase (pA-Tn5) to thereby generate DNA fragments, and isolating the DNA fragments to form the DNA library.

Also disclosed herein are methods of identifying a modification of interest in isolated DNA, the methods comprising incubating the isolated DNA with pA-Tn5 to thereby generate DNA fragments, isolating DNA fragments having the modification of interest, and identifying the modification of interest from the DNA fragments.

Also disclosed herein are kits comprising pA-Tn5 and instructions for use in performing the herein disclosed methods.

Also disclosed herein are methods of diagnosing cancer in a subject, the method comprising incubating DNA obtained from the subject with pA-Tn5 to thereby generate DNA fragments, isolating DNA fragments having a modification of interest, and identifying the position of the modification of interest from the DNA fragments, thereby diagnosing cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

The summary, as well as the following detailed description, is further understood when read in conjunction with the appended drawings. For the purpose of illustrating the disclosed methods and kits, the drawings show exemplary embodiments of the methods and kits; however, the methods and kits are not limited to the specific embodiments disclosed. In the drawings:

FIG. 1A illustrates a pA-Tn5-based methylated DNA Immunoprecipitation sequencing (MeDIP-Scq) method for analysis of methylomes of genomic DNA. The exemplary methodology shows MeDIP-Seq procedures for the analysis of DNA methylation of genomic double-stranded (ds) DNA (gMeDIP-Seq). FIG. 1B depicts the average density of gMeDIP-seq signals of eight liver cancer samples at genes with and without CpG islands at their promoters (TSS: transcription start site/TTS: transcription termination site). FIG. 1C shows a snapshot of differentially methylated regions (DMRs) at the TBX2 gene locus of three liver cancer samples and their corresponding adjacent normal tissue samples. FIG. 1D shows a heatmap of DMRs between liver tumor and adjacent normal tissues. Z score represents the log 2 (RPKM) value of gMeDIP-Seq signals. FIG. 1E illustrates the overlaps between DMRs in liver cancer identified by gMeDIP-seq in this study and those from TCGA tumor samples by 450K methylation arrays. Violin plots represent the random distribution of overlaps from 100 permutations (one sided P values were computed using random permutation distribution). Diamonds represent observed overlap between DMRs identified from TCGA liver tumors and DMRs identified by gMeDIP-Seq.

FIG. 2A illustrates a new ss-cfMeDIP-Seq procedure to analyze methylomes of cell free DNA (cfDNA). The exemplary ss-cfMeDIP-Seq method depicts the analysis of cfDNA methylomes. As the method starts with single-stranded (ss) DNA tailing and can detect DNA methylation in strand-specific (ss) manner, it is referred to herein as ss-cfMeDIP-Seq. Furthermore, this method can analyze DNA methylation of dsDNA, ssDNA and damaged DNA presented in μl cell free DNA. FIG. 2B shows the average ss-cfMeDIP-seq signals (n=10) at genes from TSS to TTS with and without CpG islands at their promoters (TSS: transcription start site/TTS: transcription termination site. FIG. 2C shows a snapshot ss-cfMeDIP-seq signals at the TBX2 gene locus from two liver cancer plasma and two control plasma samples. Sequence reads at this locus from the two inputs without methylated DNA immunoprecipitation are also shown. FIG. 2D is a heatmap of cfDNA DMRs identified between liver cancer patients and healthy controls. The Z score represents log 2 (RPKM) of ss-cfMeDIP-seq signals. FIG. 2E illustrates the overlaps between cfDNA DMRs from liver cancer patients with DMRs of liver tumor DNA samples identified in this study using gMeDIP-seq. Violin plots represent the random distribution of overlaps from 100 permutations (one sided P values were computed using random permutation distribution). Diamonds represent observed overlaps. FIG. 2F illustrates the overlaps between cfDNA DMRs from liver cancer patients with DMRs of liver tumor DNA samples identified in this study as well as using independent cohort of liver tumor samples from the TCGA dataset. Violin plots represent the random distribution of overlaps from 100 permutations (one sided P values were computed using random permutation distribution). Diamonds represent observed overlaps.

FIG. 3A depicts a workflow for the prediction of cancer types by cfDNA methylomes using the Deep Neural Network (DNN) model. Methylomes of 87 cfDNA samples from three groups of individuals (controls, brain and liver cancer patients) were analyzed. ss-cfMeDIP-seq datasets from 46 samples were used as the training cohort with the remaining 41 samples as the independent validation cohort. The training cohort was used for DMR selection and model training, yielding 10 subsets of DNN models for each sample group. The validation cohort was then evaluated by each of the 10 DNN models, with the average probability from the 10 trials reported. FIG. 3B shows a heatmap of top 600 DMRs from three groups of samples including 100 hyper- and 100 hypo-methylated DMRs from each group chosen for prediction. Z score is log 2 (RPKM) of ss-cfMeDIP-seq signals. FIG. 3C is an evaluation of prediction performances on the validation cohort using the ROC curve. Arca under each ROC curve for brain and liver cancer patients and controls are 0.95, 0.94, and 0.96, respectively, with the best sensitivity and specificity labeled as a dot on the curve. FIG. 3D illustrates the average prediction probability of each group of samples. Each column represents the group of validation samples, and each rows representing model prediction probabilities. Bars represent the probability of samples being from brain cancer, liver cancer, and healthy controls.

FIG. 4A shows the detection of brain cancer subtypes, IDH wild type and IDH mutant tumors using the validation cohort and four-class (controls, liver cancer, IDH wild type glioma, and IDH mutant glioma) DNN model. Area under each ROC curve and the best sensitivity and specificity (denoted with a dot) were indicated on the curve. FIG. 4B illustrates the average prediction probability of control, liver cancer, IDH wild type glioma and IDH mutant glioma based on four-class DNN, with bars representing the probability of IDH mutant glioma, IDH wild type glioma, liver cancer, and controls. FIG. 4C depicts the detection of early stage liver cancer using cfDNA methylomes. The ss-cfMeDIP-Seq data of liver cancer samples from the validation cohort were divided into early and late stage. The DNN model was used to evaluate these samples independently. FIG. 4D depicts the detection of late stage liver cancer using cfDNA methylomes. The ss-cfMeDIP-Seq data of liver cancer samples from the validation cohort were divided into early and late stage. The DNN model was used to evaluate these samples independently.

FIG. 5A is a prediction of brain cancer using the same ss-cfMeDIP-seq datasets with different sequence depth. Area under the ROC, specificity and sensitivity were evaluated using ss-MeDIP-Seq datasets with different sequence depth (2M, 4M, 6M, 8M, 10M reads) generated by random sampling of sequencing reads. FIG. 5B is a prediction of liver cancer using the same ss-cfMeDIP-seq datasets with different sequence depth. Area under the ROC, specificity and sensitivity were evaluated using ss-MeDIP-Seq datasets with different sequence depth (2M, 4M, 6M, 8M, 10M reads) generated by random sampling of sequencing reads. FIG. 5C is a prediction of healthy controls using the same ss-cfMeDIP-seq datasets with different sequence depth. Arca under the ROC, specificity and sensitivity were evaluated using ss-MeDIP-Seq datasets with different sequence depth (2M, 4M, 6M, 8M, 10M reads) generated by random sampling of sequencing reads. FIG. 5D illustrates a correlation analysis using ss-cfMeDIP-Seq signals on CpG islands genome wide from the healthy control, with correlation coefficients shown and represented by shade. cfDNAs from one healthy control were used for the generation of ss-cfMeDIP-Seq datasets. FIG. 5E illustrates a correlation analysis using ss-cfMeDIP-Seq signals on CpG islands genome wide from brain cancer, with correlation coefficients shown and represented by shade. cfDNAs from one brain cancer patient were used for the generation of ss-cfMeDIP-Seq datasets. FIG. 5F illustrates a correlation analysis using ss-cfMeDIP-Seq signals on CpG islands genome wide from liver cancer, with correlation coefficients shown and represented by shade. cfDNAs from one liver cancer patient was used for the generation of ss-cfMeDIP-Seq datasets. FIG. 5G shows the prediction probability of the control individual using ss-cfMeDIP-Seq datasets generated with indicated amount of cfDNAs. Bar shading represents the probability of brain cancer or, liver cancer or control. FIG. 5H shows the prediction probability of the patient with brain cancer using ss-cfMeDIP-Seq datasets generated with indicated amount of cfDNAs. Bar shading represents the probability of brain cancer or, liver cancer or control. FIG. 5I shows the prediction probability of the patient with liver cancer using ss-cfMeDIP-Seq datasets generated with indicated amount of cfDNAs. Bar shading represents the probability of brain cancer or, liver cancer or control.

FIG. 6A depicts an outline to identify genes having at least one liver cancer specific cfDNA DMR within 20 Kb from their promoters and whose expression in TCGA liver tumors being associated with patient survival. FIG. 6B illustrates ss-cfMeDIP-seq density at DMRs close to the 216 genes whose expression in liver cancer being associated with patient survival. The z-score is log 2 (RPKM) of ss-cfMeDIP-Seq signals. For these data, a “tumor suppressor gene” is a gene with at least one hypermethylated cfDNA DMR nearby and its low expression in liver tumors being associated with poor survival. For these data, an “Oncogene” is a gene with a hypomethylated cfDNA DMR nearby and its high expression in liver tumors is associated with poor survival. FIG. 6C shows the classification of 371 liver tumors in the TCGA-LIHC cohort based on expression of the 216 marker genes identified above. Patients are classified into three clusters. The shade represents the z-score of log 2 (RPKM) of RNA-seq of 216 genes in liver cancer samples. FIG. 6D illustrates Kaplan-Meier survival analysis of 371 liver cancer patients in three different clusters defined by the expression of 216 genes. P value is calculated by log rank test. FIG. 6E shows ss-cfMeDIP-Seq density at 73 DMRs that are also identified in liver tumor tissue using gMeDIP-Seq. The shade represents the z-score of log 2 (RPKM) of ss-cfMeDIP-Seq in the training cohort of 41 samples. FIG. 6F shows ss-cfMeDIP-Seq density at 73 DMRs that are also identified in liver tumor tissue using gMeDIP-Seq. The shade represents the z-score of log 2 (RPKM) of ss-cfMeDIP-Seq in the training cohort of 41 samples. FIG. 6G illustrates the classification of 371 liver tumors in TCGA-LIHC dataset based on the expression of 73 genes. FIG. 6H depicts Kaplan-Meier survival analysis of 371 liver cancer patients in different clusters defined by the expression of 73 genes. P value is calculated by log rank test.

FIG. 7A shows a snapshot of gMeDIP-Seq signals close to the DMR at the TBX2 gene locus of three liver tumor samples with their corresponding inputs without methylated DNA immunoprecipitation shown. FIG. 7B illustrates gMeDIP-Seq signals at different CpG related regions. Data are represented as mean+SEM of eight liver tumor, their corresponding adjacent normal tissues and six brain tumor samples. FIG. 7C shows the average density of gMeDIP-Seq signals of six brain tumors at genes with and without CpG islands at their promoters. Data are represented as mean±95% confidence interval. (TSS: transcription start site/TTS: transcription termination site).

FIG. 8A shows the average ss-cfMeDIP-Seq signals of 10 samples from controls surrounding TSS and TTS of genes with and without CpG islands at their promoters. Data are represented as mean±95% confidence interval. (TSS: transcription start site/TTS: transcription termination site). FIG. 8B shows the average ss-cfMeDIP-Seq signals of 10 samples from individuals with brain tumors surrounding TSS and TTS of genes with and without CpG islands at their promoters. Data are represented as mean±95% confidence interval. (TSS: transcription start site/TTS: transcription termination site). FIG. 8C illustrates ss-cfMeDIP-Seq signals at different regions related to CpG islands. Data are represented as the mean+SEM of ss-cfMeDIP-Seq signals of 10 samples from each group (liver cancer, brain cancer and healthy controls).

FIG. 9A shows the training process for the deep neural network (DNN) model. Parameters (accuracy, AUC and loss function) that gauge the performance are plotted along with the epoch number. Lines represent training datasets and cross-validations. The parameters for model performance indicated are improving with the increase of epochs. FIG. 9B illustrates the training process for the Random Forest (RF) model. Model performance (accuracy) is tuned over by the number of randomly selected features. FIG. 9C shows the training process for a GLMnet model. Model accuracy is tuned by the parameters, λ and α, where λ indicates the regularization penalty and a indicates the model mixture between ridge and lasso regressions.

FIG. 10A shows the analysis of 46 samples in the training cohort using the DNN models. The median area under the receiver operator characteristic curve (AUROC) value: brain cancer=0.99, liver cancer=0.98 and controls=1. FIG. 10B shows the analysis of 46 samples in the training cohort using the RF models. The median AUROC value: brain cancer=0.99, liver cancer=0.98, and controls=1. FIG. 10C shows the analysis of 46 samples in the training cohort using the GLMnet models. The median AUROC value: brain cancer=1, liver cancer=0.99, controls=1. FIG. 10D shows the analysis of 41 samples in the validation cohort using the DNN models. The median AUROC value: brain cancer=0.92, liver cancer=0.88, controls=0.96. FIG. 10E shows the analysis of 41 samples in the validation cohort using the RF models. The median AUROC value: brain cancer=0.91, liver cancer=0.88, controls=0.96. FIG. 10F shows the analysis of 41 samples in the validation cohort using the GLMnet models. The median AUROC value: brain cancer=0.92, liver cancer=0.88, controls=0.96. FIG. 10G illustrates ROC curves for the analysis of 41 samples in the validation cohort using the RF model with dots representing the best sensitivity and specificity. FIG. 10H illustrates ROC curves for the analysis of 41 samples in the validation cohort using the GLMnet model. Dots represent the best sensitivity and specificity.

FIG. 11A, FIG. 11B, FIG. 11C, FIG. 11D, and FIG. 11E show the impact of ss-cfMeDIP-Seq sequence depth on tumor detection using the DNN models with 10M, 8M, 6M, 4M, and 2M reads, respectively. ss-cfMeDIP-Seq datasets of 20 samples each with at least 10 million reads in the validation cohorts were chosen. ss-cfMeDIP-Seq reads of 10M, 8M, 6M, 4M, and 2M from each the 20 samples were randomly selected to generate validation cohorts with different ss-cfMeDIP-Seq reads. The DNN models were then applied to analyze these 20 samples with different sequence reads. The best sensitivity and specificity for each analysis is labeled as a dot on the ROC curves.

FIG. 12A illustrates gene identification with a nearby cfDNA DMR in liver cancer and whose expression in tumor tissue is associated with patient survival. ss-cfMeDIP-Seq signals at the CENPM gene locus from three liver cancer, three brain tumor and three controls. A hypomethylated cfDNA DMR (highlighted) was also detected at this gene locus in liver cancer tissue. FIG. 12B shows a Kaplan-Meier survival analysis of 371 liver cancer patients based on the expression of CENPM in patient samples. P value is calculated by log rank test. FIG. 12C illustrates gene identification with a nearby cfDNA DMR in liver cancer and whose expression in tumor tissue is associated with patient survival. A hypermethylated cfDNA DMR (highlighted) is detected in the GNA14 gene locus in samples from liver cancer cases compared to controls or patients with brain cancer. FIG. 12D shows a Kaplan-Meier survival analysis of 371 liver cancer patients based on the expression of GNA14. P value is calculated by log rank test.

FIG. 13A illustrates ss-cfMeDIP-Seq signals at the HOXB2 gene locus, with the hypomethylated DMR for three brain tumor samples (shading) compared to three liver cancer and three normal controls. FIG. 13B shows a Kaplan-Meier survival analysis of 154 brain cancer patients in TCGA database based on the expression of HOXB2 in the tumor samples. P value is calculated by log rank test. FIG. 13C illustrates ss-cfMeDIP-Seq signals at the AEBP1 gene locus, with the hypomethylated DMR for three brain cancer patients highlighted. FIG. 13D shows a Kaplan-Meier survival analysis of 154 brain cancer patients in TCGA database based on the expression of AEBP1. P value is calculated by log rank test. FIG. 13E illustrates ss-cfMcDIP-seq density at the 422 genes with at least one cfDNA DMR nearby and whose expression in brain tumors being associated with patient survival. The z-score is log 2 (RPKM) of ss-cfMeDIP-Seq signals. For these data, a “tumor suppressor gene” is a gene with at least one hypermethylated cfDNA DMR nearby and its low expression in liver tumors being associated with poor survival. For these data, an “Oncogene” is a gene with a hypomethylated cfDNA DMR nearby and its high expression in liver tumors is associated with poor survival. FIG. 13F shows that 154 brain cancer patients in TCGA-GBM cohort can be classified into three clusters based on the expression of 422 genes in their tumors. The shade represents the z-score of log 2 (RPKM) of 422 genes. Glioma samples with IDH mutations were largely found in Cluster 2. FIG. 13G shows a Kaplan-Meier survival analysis of 154 brain cancer patients in different clusters based on the expression of 422 genes in TCGA database. P value is calculated by log rank test. cfDNA DMRs from brain tumor samples likely reflect genes whose expression in brain tumors being associated with patient survival.

FIG. 14A illustrates an outline of ssg-MeDIP-Seq procedures for the analysis of DNA methylation of genomic DNA in a strand-specific manner. FIG. 14B depicts a snapshot of liver tumor DNA DMR at the TBX2 gene locus of three liver cancer samples and their corresponding adjacent non-tumor (Adj-NT) tissue samples. FIG. 14C shows heatmap of DMRs between 8 liver tumor and corresponding Adj-NT. Z score represents the log 2 (RPKM) value of ssg-MeDIP-Seq signals. FIG. 14D illustrates overlaps between liver cancer DMRs identified by ssg-MeDIP-Seq in this study and those from TCGA tumor samples by 450K methylation arrays. Violin plots represent the random distribution of overlaps from 100 permutations (one sided P values were computed using random permutation distribution). Diamonds represent observed overlap between DMRs identified from TCGA liver tumors and DMRs identified by ssg-MeDIP-Seq. FIG. 14E illustrates the sequence element enrichment of liver tumor DNA DMRs. The DMRs were first overlapped with each annotated locus and compared with the overlapped number in random distribution for the calculation of the Z score. The significantly enriched sequence elements were labelled with asterisks for hyper-methylated DMR and blue hypo-methylated DMR. LINE, Long Interspersed Nuclear Element retrotransposons; LTR, long-terminal repeat; SINE, short interspersed nuclear element; DNA, DNA transposon.

FIG. 15A is an illustration of symmetric methylation and hemi-methylation. Symmetric methylation refers to DNA methylation at CpG dinucleotides of both Watson and Crick strands equally, where a hemi-methylation region (HMR) refers to preferential methylation of CpG dinucleotides at one strand over the other strand. FIG. 15B depicts a snapshot of tumor DNA differentially hemi-methylation region (DHMR) at the C1QTNF4 gene locus in two liver tumor samples, but not in their corresponding Adj-NT controls. FIG. 15C illustrates the enrichment for liver tumor DNA HMRs. The HMRs, with the cut off of bias score over 0.3, was overlapped with each annotated locus. The Z score was calculated by compared with the overlapped number in random distribution. The significantly enriched sequence elements was labelled with asterisks, with HMRs for liver tumor and Adj-NT DNA shown. FIG. 15D shows a heatmap of total 12,612 DHMRs from 8 liver tumor samples compared to their corresponding Adj-NT controls. Hemi-methylation level is shown from −1 to 1, with 4,686 liver tumor DNA DHMRs showing increased hemi-methylation at either Watson or Crick strand, and 7926 DHMRs with reduced hemi-methylation compared to controls. FIG. 15E depicts overlap of DMRs and DHMRs of eight liver tumor samples compared to their corresponding Adj-NT controls. FIG. 15F illustrates the enrichment for liver tumor DNA DHMRs of increased and reduced hemi-methylation compared to controls samples. FIG. 15G illustrates the GO function enrichment for genes closest to liver tumor DNA HMRs. FIG. 15H illustrates the GO function enrichment for genes closet to liver tumor DNA DHMRs compared to Adj-NT control samples. FIG. 16A shows an outline of the sscf-MeDIP-Seq method for analyzing cfDNA methylomes. FIG. 16B depicts a snapshot of cfDNA DMR at the TBX2 gene locus from two plasma cfDNA samples and two control plasma cfDNA samples. Also shown are sequence reads of two input samples in which methylated DNA immunoprecipitation were also not performed. FIG. 16C is a heatmap of cfDNA DMRs from 10 plasma samples of liver cancer and 10 plasma samples of non-tumor controls. The Z score represents log 2 (RPKM) of sscf-MeDIP-seq signals. FIG. 16D illustrates overlaps between liver tumor cfDNA DMRs identified by sscf-MeDIP-Seq and liver tumor DNA DMRs identified in this study using ssg-MeDIP-seq. Violin plots represent the random distribution of overlaps from 100 permutations (one sided P values were computed using random permutation distribution). Diamonds represent observed overlaps. FIG. 16E depicts a snapshot of cfDNA DHMR at the NAALADL2 gene locus from two liver cancer plasma samples and two control plasma samples. FIG. 16F shows a heatmap of plasma cfDNA DHMRs of 10 liver cancer samples and 10 controls. The hemi-methylation score represents hemi-methylation level. FIG. 16G depicts overlap of plasma cfDNA DMRs and cfDNA DHMRs of the same 10 liver cancer samples compared to 10 controls.

FIG. 17A illustrates a workflow of machine learning model training. Methylomes of 221 cfDNA samples from three groups of individuals (controls, brain and liver cancer patients) were analyzed using sscf-MeDIP-Seq. 175 sscf-MeDIP-seq datasets (80%) were used as the training cohort with the remaining 46 (20%) samples as the independent validation cohort. The training cohort was used for DMR and DHMR selection and training of machine learning models, results in 10 models for each sample group using DMRs or DHMRs as the input for the training. The DMR- and DHMR-based models were then further unified to build a final calibration model. The validation cohort was then evaluated using models using DMR, DHMR and DMR+DHMR as inputs. FIG. 17B, FIG. 17C, and FIG. 17D show graphs evaluating model performances for the prediction of control (FIG. 17B), liver tumor (FIG. 17C) and brain tumor (FIG. 17D) cfDNA samples in the validation cohort using DMR, DHMR, or DMR+DHMR models. The best sensitivity and specificity point were marked with a dot: control cfDNA samples: specificity=94.3% and sensitivity=81.8% for DMR model; specificity=82.9% and sensitivity=72.7% for DHMR model; and specificity=88.6% and sensitivity=100% for DMR+DHMR model; liver cancer cfDNA samples: DMR-based model: specificity=80.6% and sensitivity=100%; DHMR-based models: specificity=90.3% and sensitivity=86.7%; DMR+DHMR-based models: specificity=93.5% and sensitivity=93.3%; brain tumor cfDNA samples: DMR model: specificity=100% and sensitivity=85.0%; DHMR models; specificity=92.3% and sensitivity=70.0%; DMR+DHMR models: specificity=100% and sensitivity=90.0%. FIG. 17E illustrates the average prediction probability of each group of samples using models trained with DMRs+DHMRs. Each column represents the group of validation samples, and each rows representing model predictions. Bars represent probability of samples being from brain cancer, liver cancer, and healthy controls.

FIG. 18A illustrates a workflow for building brain tumor subtype models. Models for the IDH WT and IDH mutant gliomas were first trained by DMRs and DHMRs identified using the training cohort samples as the inputs and then combined with the three class models (controls, liver and brain tumor) based on the Bayes' theorem to derive models for predicting four sample groups of the IDH WT and IDH mutant brain tumor, liver tumor and control samples. FIG. 18B and FIG. 18C show graphs evaluating predicting IDH mutant (FIG. 18B) and IDH wild type (FIG. 18C) brain cancer samples in validation cohort using models trained with DMRs, DHMRs, DMRs+DHMRs. The best sensitivity and specificity point are labeled as dots on the curve: IDH mutant brain tumors: DMR models: specificity=100% and sensitivity=63.6%; DHMR models: specificity=77.1% and sensitivity=81.8%; DMR+DHMR models: specificity=82.9% and sensitivity=90.9%. IDH WT brain tumors: DMR models: specificity=94.6% and sensitivity=100%; DHMR models: specificity=78.4% and sensitivity=77.8%; DMR+DHMR models: specificity=97.3% and sensitivity=100%. FIG. 18D illustrates the average prediction probability of each group of samples. Each column represents the sample groups in the validation cohort, and each rows representing model predictions. Bars represent probability of samples being from brain IDH mutant cancer, brain IDH WT cancer, liver cancer, and healthy controls.

FIG. 19A depicts an exemplary outline to identify genes having at least one liver cancer cfDNA DMR or DHMR within 20 Kb from their promoters and whose expression in TCGA liver tumor tissue samples being associated with patient survival. FIG. 19B illustrates sscf-MeDIP-seq density at DMRs close to the 78 genes with at least one cfDNA DMR nearby. The z-score is log 2 (RPKM) of sscf-MeDIP-Seq signals. A “Hyper DMR” refers to a gene with at least one hyper-methylated cfDNA DMR nearby, A “Hypo DMR” is defined as a gene with a hypo-methylated cfDNA DMR nearby. FIG. 19C shows the classification of 371 liver tumors in the TCGA-LIHC cohort based on expression of the 78 marker genes identified above. Patients are classified into two clusters. Shown is the z-score of log 2 (RPKM) of RNA-seq signals of 78 genes in 371 liver cancer samples. FIG. 19D illustrates Kaplan-Meier survival analysis of 371 liver cancer patients separated into two clusters as in c. P value is calculated by log rank test. FIG. 19E illustrates liver tumor cfDNA DHMRs at the 72 genes with at least one DHMR nearby and whose expression in liver cancer tissue samples being associated with patient survival. The hemi-methylation ranges from −1 to 1. FIG. 19F shows the classification of 371 liver tumor samples in the TCGA-LIHC cohort based on expression of the 72 marker genes close to cfDNA DHMRs. Shown is the z-score of log 2 (RPKM) of RNA-seq signals of 72 genes in liver cancer samples. FIG. 19G illustrates Kaplan-Meier survival analysis of 371 liver cancer patients separated into two clusters based on analysis in j. P value is calculated by log rank test.

FIG. 20A illustrates the average genomic DNA methylation density surrounding genes (3000 bp upstream of transcription start site (TSS) and 3000 bp downstream transcription termination site (TTS) in eight liver tumor samples measured by ssg-MeDIP-Seq. FIG. 20B, FIG. 20C, and FIG. 20D illustrate the average cfDNA methylation density surrounding genes in 10 control samples (FIG. 20B), 10 liver cancer samples (FIG. 20C) and 10 brain tumor samples (FIG. 20D) based on sscf-MeDIP-Seq analysis. Genes are grouped those with (13,553) and without (6,713) CpG islands at their promoters. TSS: transcription start site, TTS: transcription termination site. Data are represented as mean±95% confidence interval.

FIG. 21A, FIG. 21B, and FIG. 21C are graphs showing the enrichment of liver cancer cfDNA DMR (FIG. 21A), cfDNA HMR (FIG. 21B) and liver cancer cfDNA DHMR (FIG. 21C) compared to controls. In FIG. 21A, hyper- and hypo-differentially methylated regions of liver cancer cfDNA are annotated separately. In FIG. 21B, hemi-methylated regions (HMR) of liver cancer cfDNA and control cfDNA samples are annotated separately. In FIG. 21C, differentially hemi-methylated regions (DHMR) of liver cancer cfDNA samples compared to control samples with increased and reduced hemi-methylation are annotated separately. The interested region is firstly overlapped with each annotated locus, then compared with the overlapped number in random distribution and calculated the Z score. The significantly enriched locus is labelled with asterisks. FIG. 21D illustrates overlaps between liver cancer cfDNA DHMRs and liver cancer DNA DHMRs. cfDNA DHMRs were identified using control cfDNA samples, and tumor DNA DHMRs identified using corresponding Adj-NT tissue DNA, respectively. Violin plots represent the random distribution of overlaps from 100 permutations (one sided P values were computed using random permutation distribution) with diamonds being observed overlaps. FIG. 21E illustrates overlaps between liver cancer cfDNA DHMRs and liver cancer DNA DHMRs with both cfDNA and tumor DNA DHMR identified using the same control cfDNA samples.

FIG. 22A, FIG. 22B, and FIG. 22C are graphs evaluating the performance of random forest models in validation cohort using the ROC curve on the control samples (FIG. 22A), liver cancer samples (FIG. 22B) and brain cancer samples (FIG. 22C) using cfDNA DMR, DHMR or DMR+DHMR as inputs for modeling training. FIG. 22D, FIG. 22E, and FIG. 22F are graphs evaluating the performance of deep neural network models for prediction of control cfDNA samples (FIG. 22D), liver cancer cfDNA samples (FIG. 22E) and brain cancer cfDNA samples (FIG. 22F) in the validation cohort. Models were trained using DMRs, DHMRs or DMRs+DHMRs. FIG. 22G depicts AUROC values of the validation cohort samples predicted using GLMNET models trained with DMRs using different cutoff parameters: p=0.05, LFC=0.58; p=0.05, LFC=1; p=0.01, LFC=1. LFC: log fold changes in DNA methylation density based on sscf-MeDIP-Seq signals. FIG. 22H depicts AUROC values of different GLMNET models trained using DHMRs selected from different cutoffs: p=0.01, feature importance=0; p=0.01, feature importance=30; p=0.01, feature importance=50. FIG. 22I depicts AUROC values of the validation cohort samples predicted using GLMNET models trained with DMR and DHMRs with different cutoffs: parameters 1: DMR p=0.05, LFC=0.58, DHMR p=0.01, importance=0; parameters2: DMR p=0.05, LFC=0.58, DHMR p=0.01, importance=50; parameters3: DMR p=0.01, LFC=1, DHMR p=0.01, importance=0; parameters4: DMR p=0.01, LFC=1, DHMR p=0.01, importance=50.

FIG. 23A, FIG. 23B, and FIG. 23C illustrate the prediction probability using sscf-MeDIP-Seq datasets generated from different amount of cfDNA from one control cfDNA sample (FIG. 23A), one liver cancer cfDNA sample (FIG. 23B) and one brain cancer cfDNA sample (FIG. 23C). Shadings represents the probability of brain cancer, liver cancer or healthy normal.

FIG. 24A depicts a snapshot of hypomethylated DMR surrounding SOX14 gene in liver cancer compared to control and brain tumor samples. FIG. 24B illustrates survival analysis of 371 liver cancer samples in the TCGA database separated into two groups based on the median expression of SOX14 in these 371 samples. FIG. 24C depicts a snapshot of a liver cancer cfDNA DHMR with increased hemi-methylation surrounding the PATE3 gene compared to control and brain tumor samples. FIG. 24D illustrates survival of two groups of patients separated based on the median expression of PATE3. FIG. 24E illustrates overlap of 78 genes with at least one cfDNA DMR nearby and 72 genes with at least cfDNA DHMR nearby and the expression of these genes in liver cancer tissues is associated with patient survival.

FIG. 25A depicts a snapshot of brain cancer cfDNA hypomethylated DMR at the TRIM56 gene locus. FIG. 25B illustrates survival analysis of 156 patients separated into two groups based on the median expression of TRIM56. FIG. 25C depicts a snapshot of brain cancer cfDNA DHMR with increased hemi-methylation at the BET1 gene locus. FIG. 25D illustrates survival analysis of 156 brain tumor patients separated into two groups based on the median expression of BET1. FIG. 25E illustrates overlap of 61 genes identified using cfDNA DMR and 17 genes identified using cfDNA DHMR in brain cancer. These genes have at least one cfDNA DMR or DHMR nearby and the expression of each of these genes in brain tumor tissues is associated with patient survival.

FIG. 26A illustrates cfDNA methylation density at the 61 genes with at least one brain cancer cfDNA DMR nearby and with their expression in brain cancer tissue being associated with patient survival. The z-score is log 2 (RPKM) of sscf-MeDIP-Seq signals. A “Hyper DMR gene” refers to a gene with at least one hyper-methylated cfDNA DMR nearby. A “Hypo DMR gene” is defined as a gene with a hypo-methylated cfDNA DMR nearby. FIG. 26B shows the classification of 156 brain tumor samples in the TCGA-GBM cohort based on expression of the 61 genes identified above. Shown is the z-score of log 2 (RPKM) of 61 genes in brain cancer samples based on RNA-seq. FIG. 26C illustrates Kaplan-Meier survival analysis of 156 brain cancer patients in two clusters defined by the expression of 61 genes. P value is calculated by log rank test. FIG. 26D illustrates cfDNA hemi-methylation at 17 genes with at least one brain cancer cfDNA nearby and with their expression in brain cancer tissues being associated with patient survival. Hemi-methylation is ranged from −1 to 1. A “Reduced DHMR gene” refers to a gene with at least one cfDNA DHMR with reduced hemi-methylation nearby. An “Increased DHMR gene” is defined as a gene with a cfDNA DHMR with increased hemimethylation nearby. FIG. 26E shows the Classification of 156 brain tumor samples in the TCGA-GBM cohort based on expression of the 17 marker genes identified by DHMRs. Shown is the z-score of log 2 (RPKM) of RNA-seq of 17 genes in brain cancer samples. FIG. 26F illustrates Kaplan-Meier survival analysis of 156 brain cancer patients in two clusters defined by the expression of 17 genes shown in e. P value is calculated by log rank test.

### DETAILED DESCRIPTION OF ILLUSTRATIVE EMBODIMENTS

The disclosed methods and kits may be understood more readily by reference to the following detailed description taken in connection with the accompanying figures, which form a part of this disclosure. It is to be understood that the disclosed methods and kits are not limited to the specific methods and kits described and/or shown herein, and that the terminology used herein is for the purpose of describing particular embodiments by way of example only and is not intended to be limiting of the claimed methods and kits.

Unless specifically stated otherwise, any description as to a possible mechanism or mode of action or reason for improvement is meant to be illustrative only, and the disclosed methods and kits are not to be constrained by the correctness or incorrectness of any such suggested mechanism or mode of action or reason for improvement.

Throughout this text, the descriptions refer to methods and kits. Where the disclosure describes or claims a feature or embodiment associated with the methods, such a feature or embodiment is equally applicable to the kits. Likewise, where the disclosure describes or claims a feature or embodiment associated with the kits, such a feature or embodiment is equally applicable to the methods.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed methods which are, for clarity, described herein in the context of separate embodiments, may also be provided in combination in a single embodiment. Conversely, various features of the disclosed methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

The term “about” when used in reference to numerical ranges, cutoffs, or specific values is used to indicate that the recited values may vary by up to as much as 10% from the listed value. Thus, the term “about” is used to encompass variations of #10% or less, variations of ±5% or less, variations of ±1% or less, variations of ±0.5% or less, or variations of ±0.1% or less from the specified value.

Disclosed herein are methods of preparing a DNA library from isolated DNA comprise incubating the isolated DNA with a fusion protein comprising protein A and a Tn5 transposase (pA-Tn5) to thereby generate DNA fragments, and isolating the DNA fragments to form the DNA library.

In some embodiments, the DNA library contains DNA fragments that are about 100 nucleotides to about 300 nucleotides in length. In some embodiments, the DNA library contains DNA fragments that are about 100 nucleotides in length. In some embodiments, the DNA library contains DNA fragments that are about 200 nucleotides in length. In some embodiments, the DNA library contains DNA fragments that are about 300 nucleotides in length.

About 100 ng of DNA can be incubated with the pA-Tn5 to prepare the DNA library. In some embodiments, about 100 ng of isolated DNA is incubated with the pA-Tn5.

The pA-Tn5 can attach one or more tags to the DNA fragments. The tag can be attached to both strands of the DNA fragments. In some embodiments, the pA-Tn5 attaches a first tag to the 5′ end of the DNA fragments. The pA-Tn5 can attach a first tag to the 5′ end of both strands of a DNA fragment. The tag can be a DNA sequence for Tn5 binding and/or a primer for polymerase chain reaction (PCR). In some embodiments, a second tag is ligated to the 3′ end of the DNA fragments though oligo-replacement. The oligo-replacement can comprise replacing a 3′ tag on each strand of the isolated DNA fragment with a different tag. The 3′ tag can be replaced with oligo-replacement as described or incorporated herein.

The isolated DNA can be obtained from any source containing suitable DNA.

Isolated DNA includes, for example, DNA isolated from cells or patients samples, such as from tissues or bodily fluids. In some embodiments, the isolated DNA is genomic DNA. In some embodiments, the isolated DNA is from a cell.

The pA-Tn5 can be used in the absence of an antibody. In some embodiments, the method does not use an antibody or other DNA-targeting molecule to target the pA-Tn5 to the DNA.

The methods can further comprise amplifying one or more of the DNA fragments from the DNA library using polymerase chain reaction (PCR). In some embodiments, the methods of amplifying the DNA fragments further comprise sequencing the amplified DNA fragments.

Also disclosed herein are methods of identifying a modification of interest in isolated DNA, the method comprises incubating the isolated DNA with pA-Tn5 to thereby generate DNA fragments, isolating DNA fragments having the modification of interest, and identifying the modification of interest from the DNA fragments.

The DNA fragments having the modification of interest can be isolated via any suitable method, including for example, methods utilizing an antibody that binds the modification of interest. In some embodiments, isolating the DNA fragments having the modification of interest comprises immunoprecipitating the DNA fragments having the modification of interest with an antibody. The disclosed methods can be used for identifying, for example, methylation, hydroxymethylation, formylation, and carboxylation. Consequently, the antibody used for isolation can be an anti-methylation-specific antibody, an anti-hydroxymethylation-specific antibody, an anti-formylation-specific antibody, or an anti-carboxylation-specific antibody. In some embodiments, the antibody is an anti-methylation-specific antibody. Suitable methylation-specific antibodies include, for example, an anti-5-methylcytosine (5mC) antibody.

The identifying can comprise amplifying immunoprecipitated DNA fragments using polymerase chain reaction (PCR). In some embodiments, the identifying comprises sequencing the immunoprecipitated DNA fragments.

The pA-Tn5 can attach one or more tags to the DNA fragments. The tag can be attached to both strands of the DNA fragments. In some embodiments, the pA-Tn5 attaches a first tag to the 5′ end of the DNA fragments. The pA-Tn5 can attach a first tag to the 5′ end of both strands of a DNA fragment. The tag can be a DNA sequence for Tn5 binding and/or a primer for polymerase chain reaction (PCR). In some embodiments, a second tag is ligated to the 3′ end of the DNA fragments though oligo-replacement. The oligo-replacement can comprise replacing a 3′ tag on each strand of the isolated DNA fragment with a different tag. The 3′ tag can be replaced with oligo-replacement as described or incorporated herein.

The methods of identifying a modification of interest in isolated DNA can further comprise denaturing the DNA fragments into single stranded DNA fragments prior to immunoprecipitation.

In some embodiments, the DNA fragments having the modification of interest are about 100 nucleotides to about 300 nucleotides in length. In some embodiments, the DNA fragments are about 100 nucleotides in length. In some embodiments, the DNA fragments are about 200 nucleotides in length. In some embodiments, the DNA fragments are about 300 nucleotides in length.

About 100 ng of DNA can be incubated with the pA-Tn5 to generate DNA fragments having the modification of interest. In some embodiments, about 100 ng of isolated DNA is incubated with the pA-Tn5. In some embodiments, the isolated DNA is genomic DNA. In some embodiments, the isolated DNA is from a cell.

The pA-Tn5 can be used in the absence of an antibody. In some embodiments, the method does not use an antibody or other DNA-targeting molecule to target the pA-Tn5 to the DNA.

Also disclosed herein are kits comprising pA-Tn5 and instructions for use in performing any of the herein disclosed methods. The instructions can be for the use of the kit in preparing a DNA library from isolated DNA using pA-Tn5. The instructions can be for the use of the kit in identifying a modification of interest in isolated DNA using pA-Tn5. In some embodiments, the kit further comprises one or more oligonucleotides. The one or more oligonucleotides can comprise oligonucleotides adapted to serve as a 3′ tag. The 3′ tag can be ligated to DNA fragments being analyzed or processed using the herein disclosed kits. The one or more oligonucleotides can comprise oligonucleotide primers. The primers can be complementary to a 5′ tag or a 3′ tag on the DNA fragments being analyzed or processed using the herein disclosed kits. The primers can be used for PCR amplification of the DNA fragments being analyzed or processed using the herein disclosed kits.

In some embodiments, the kit further comprises an antibody that binds to a DNA modification. The antibody can be an anti-methylation-specific antibody, an anti-hydroxymethylation-specific antibody, an anti-formylation-specific antibody, or an anti-carboxylation-specific antibody. In some embodiments, the antibody is an anti-methylation-specific antibody. Suitable methylation-specific antibodies include, for example, an anti-5-methylcytosine (5mC) antibody. The antibody can be used for immunoprecipitation of DNA fragments being analyzed or processed using the herein disclosed kits. In some embodiments, the immunoprecipitated DNA fragments are amplified with PCR using the one or more oligonucleotide primers that the kits can further comprise.

Also disclosed herein are methods of diagnosing cancer in a subject, the methods comprising incubating DNA obtained from the subject with pA-Tn5 to thereby generate DNA fragments, isolating DNA fragments having a modification of interest, and identifying the position of the modification of interest from the DNA fragments, thereby diagnosing cancer. In some embodiments, the isolating comprises immunoprecipitating the DNA fragments having the modification of interest with an antibody. The immunoprecipitated DNA fragments can be sequenced. The antibody can be an anti-methylation-specific antibody, an anti-hydroxymethylation-specific antibody, an anti-formylation-specific antibody, or an anti-carboxylation-specific antibody. In some embodiments, the antibody is an anti-methylation-specific antibody. Suitable methylation-specific antibodies include, for example, an anti-5-methylcytosine (5mC) antibody. In some embodiments, the identifying comprises amplifying immunoprecipitated DNA fragments with polymerase chain reaction (PCR). The identifying can comprise sequencing the immunoprecipitated DNA fragments. The DNA sequencing can occur after amplification of the DNA fragments via PCR.

The pA-Tn5 can attach one or more tags to the DNA fragments. The tag can be attached to both strands of the DNA fragments. In some embodiments, the pA-Tn5 attaches a first tag to the 5′ end of the DNA fragments. The pA-Tn5 can attach a first tag to the 5′ end of both strands of a DNA fragment. The tag can be a DNA sequence for Tn5 binding and/or a primer for polymerase chain reaction (PCR). In some embodiments, a second tag is ligated to the 3′ end of the DNA fragments though oligo-replacement. The oligo-replacement can comprise replacing a 3′ tag on each strand of the isolated DNA fragment with a different tag. The 3′ tag can be replaced with oligo-replacement as described or incorporated herein.

In some embodiments, the DNA fragments are denatured into single stranded DNA fragments prior to immunoprecipitation. The pA-Tn5 can fragment the DNA into DNA fragments that are about 100 nucleotides to about 300 nucleotides in length. In some embodiments, the DNA fragments are about 100 nucleotides in length. In some embodiments, the DNA fragments are about 200 nucleotides in length. In some embodiments, the DNA fragments are about 300 nucleotides in length.

In some embodiments, about 100 ng of DNA is incubated with the pA-Tn5. The DNA can be obtained from any source containing suitable DNA, including for example, DNA from cells or patients samples, such as from tissues or bodily fluids. In some embodiments, the DNA is genomic DNA. The DNA can be isolated from a cell from the subject. The DNA can be obtained from plasma from the subject. The DNA can be obtained from blood from the subject. In some embodiments, the method does not use an antibody or other DNA-targeting molecule to target the pA-Tn5 to the DNA.

### EXAMPLES

The following examples are provided to further describe some of the embodiments disclosed herein. The examples are intended to illustrate, not to limit, the disclosed embodiments.

### Example 1

Aberrant DNA methylation occurs early in tumorigenesis, and DNA methylomes of cancer cells and plasma cell-free DNA (cfDNA) have been used for cancer detection and classification. Here optimized methods of methylated DNA immunoprecipitation sequencing (MeDIP-Seq) are reported for genomic DNA (gMeDIP-Seq) and plasma cfDNA (ss-cfMeDIP-Seq), which allows the production of reliable MeDIP-Seq datasets from less than 100 ng genomic DNA and from cfDNA purified from about 200-300 μl of plasma samples, respectively. Using the ss-cfMeDIP-Seq method, cfDNA methylomes were analyzed of 87 samples from subjects with liver cancer, brain cancer and healthy individuals. Among them, 46 samples were chosen as the discovery cohort for the identification of differentially methylated regions (DMRs) specific in each group of subjects and for the training of Deep Neural Network (DNN) models of multi-cancer classifications. Based on the trained DNN models, the other 41 samples in the validation cohort can be classified into three groups with high accuracy. Moreover, brain tumor subtypes and early-stage liver cancer can be accurately detected in the validation cohort. Finally, based on cfDNA DMRs, a panel of signature genes were identified specific either for liver or brain cancer that are associated with patient survival. Together, these studies provide proof-of-principle for cancer detection by analyzing methylomes of plasma cfDNA using this sensitive method.

Early tumor detection has potential to improve prognosis for cancer patients because the average 5-year patient survivals for cancer detected at early and late stages are 91% and 26%, respectively. Liquid biopsy offers several advantages for cancer early detection. For instance, liquid biopsy samples can be obtained non-invasively and in principle can overcome the challenges arising from tumor heterogeneity that confounds tissue biopsy procedures. One challenge in tumor detection by liquid biopsy is the requirement for the highly sensitive assays that can detect cancer-associated alterations.

Plasma cell free DNA (cfDNA) molecules are a mixture of extracellular DNA fragments released from apoptotic and/or necrotic cells or released via active secretion. While the majority of the plasma cfDNAs comes from normal cells such as lymphocytes, cancer cells also release DNA fragments into circulation. Analysis of cancer related mutations in plasma cfDNA has been reported for early cancer detection. Due to limited mutations in cancer cells and the evolving nature of these mutations, a significant amount of test material (7.5-10 ml plasma) is required for specific mutation detection. In addition to genetic mutations, aberrant epigenetic changes have been well documented in tumorigenesis and tumor evolution. For instance, aberrant DNA methylation (5-mC) likely precedes genetic mutations during cell transformation. Furthermore, 5-mC patterns are unique to cell types. Therefore, 5-mC patterns of plasma cfDNA are likely associated with tumor cell origin.

Several methods have been developed to analyze the methylome of plasma cfDNA for tumor detection. The CCGA (Circulating Cell-Free Genome Atlas) employed targeted bisulfite sequencing to analyze methylation regions of cfDNA in more than 50 cancer types. Because bisulfite treatment of DNA results in a marked loss of DNA, this method, on average, requires up to 80 ml plasma and over 100 million sequence reads per sample for tumor detection. Shen et al. and Nassiri et al. developed a cell-free methylated DNA immunoprecipitation and high-throughput sequencing (cfMeDIP-seq) method based on double strand DNA ligation. This method requires cfDNA purified from 0.5-3.5 ml plasma samples and 30-60 million reads per sample. A large fraction of cfDNA are single-stranded (ss) DNA fragments and damaged double-stranded DNA fragments. These DNA molecules will most likely not be included in analysis when cfDNA is used in the double stranded DNA ligation method. To address this problem, a new method was developed to analyze methylomes of cfDNA based on the preparation of ssDNA libraries for next-generation sequencing, which allows for the inclusion of all DNA molecules including ssDNA, dsDNA and damaged DNA in methylation analysis. Using this method, reliable datasets were produced with cfDNA isolated from 200-300 μl of plasma and tumors were classified with high accuracy.

Moreover, differentially methylated regions (DMRs) identified with cfDNA from liver cancer patients are consistent with DMRs identified in liver cancer tissues. Furthermore, using DMRs identified from a cohort of training samples and machine learning, early-stage liver cancer and brain tumor subtypes were detected with high accuracy and specificity. Finally, cfDNA DMRs identified from subjects with liver and brain tumors likely reflect alterations in gene expression in the primary liver and brain cancer tissues, respectively, as the expression of these DMR-associated genes are associated with patient survival. Herein provided is a proof-of-principle study that cfDNA methylomes identified by the ss-cfMeDIP-seq procedures can be used for tumor detection and for the prediction of cancer prognosis.

Development of Genomic Methylated DNA Immunoprecipitation Sequencing Method (gMeDIP-Seq)

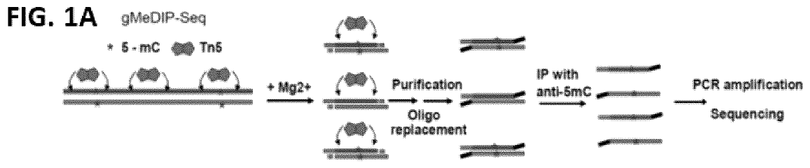

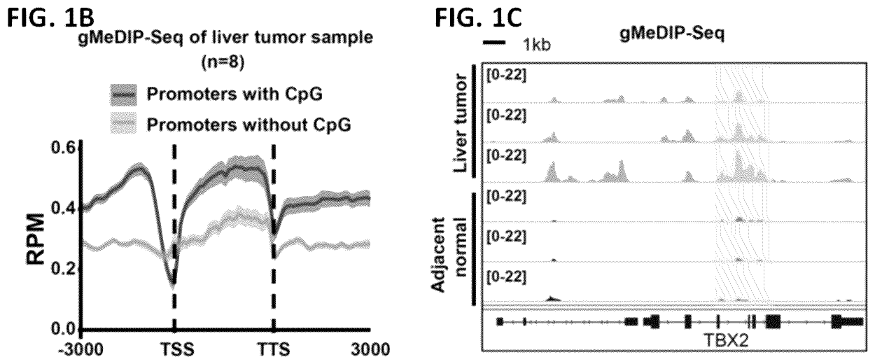

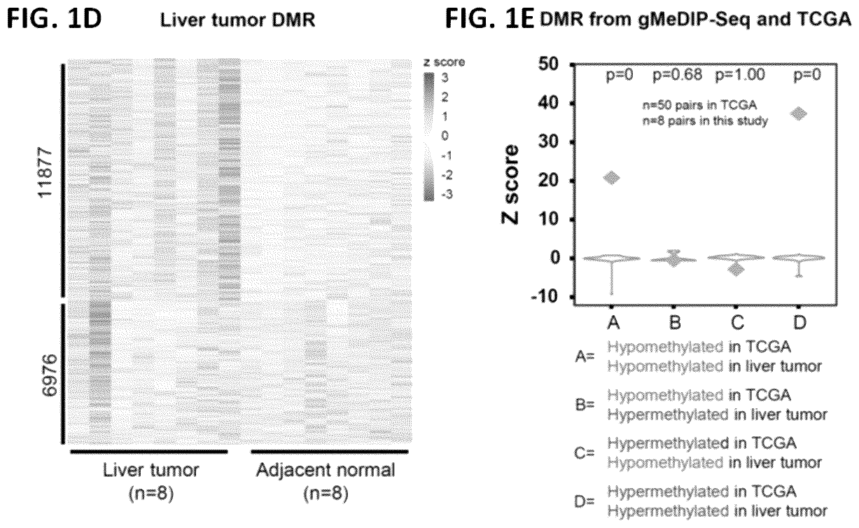

MeDIP-seq has been used to analyze DNA methylation (5-mC) in cells. Almost all published procedures rely on sonication of genomic DNA into small fragments followed by immunoprecipitation with antibodies against methylated DNA. The Tn5 transposase was tested for its ability to fragment genomic DNA before immunoprecipitation (FIG. 1A). Briefly, 100 ng of genomic DNA isolated from tissues was incubated with a fusion protein containing protein A and the Tn5 transposase (pA-Tn5), which inserts an adaptor into dsDNA in a sequence independent manner. As pA-Tn5 covalently ligates one strand of the adaptor to the target DNA, a different adaptor was ligated at the 3′ end through the oligo-replacement step. In this way, DNA methylation patterns could be analyzed in a strand-specific manner. Following the adaptor ligation, DNA fragments were denatured into single stranded DNA (ssDNA) and methylated DNAs were immunoprecipitated using antibodies against 5-mC.

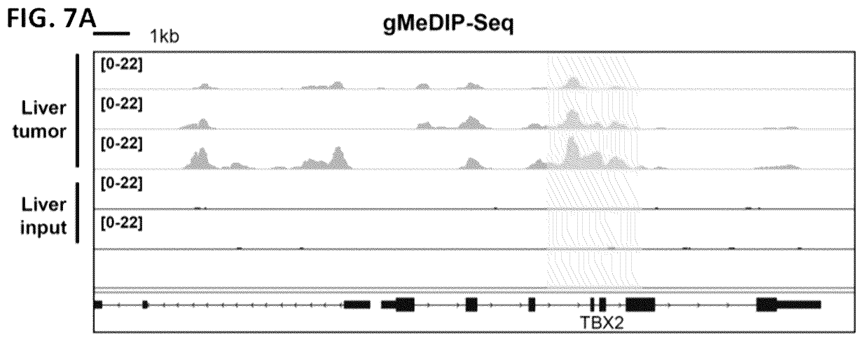

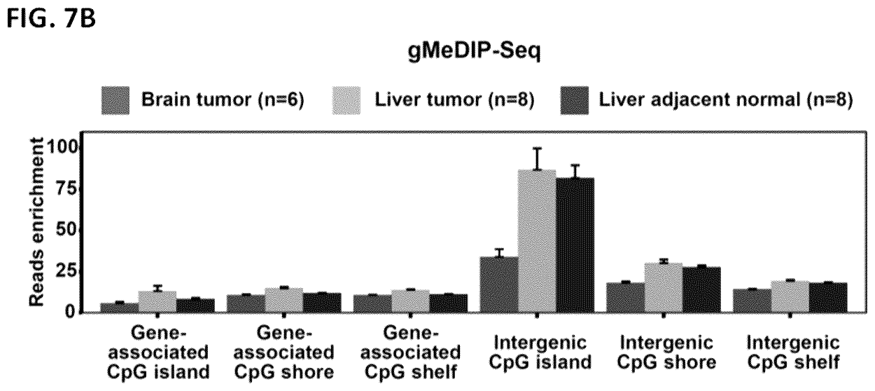

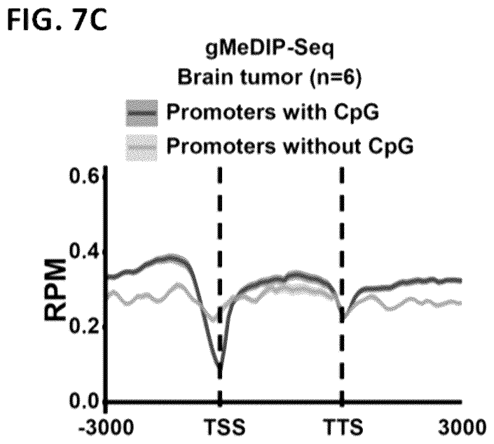

The enriched methylated ssDNAs were amplified by PCR for library preparation and subsequent sequencing (FIG. 1A). Using this method, DNA methylation of 16 tissue samples was analyzed, eight isolated from liver tumors and eight from their corresponding adjacent normal tissues. gMeDIP-seq signals were depleted at the promoters of genes with CpG islands compared to those without CpG islands (FIG. 1B). The MeDIP-seq reads enrichment was calculated at gene-associated CpG islands, CpG shores and CpG shelfs, and intergenic CpG islands, CpG shores and shelfs and it was observed that the signals are enriched at intergenic CpG islands in both liver tumor and tumor-adjacent tissues (FIG. 7A). Similar distribution patterns of DNA methylation in six brain tumors were also detected (FIG. 7B). Together, these results indicate that gMeDIP-Seq method is reliable to detect genome wide DNA methylomes.

By inspection of MeDIP-seq signals at the gene locus of TBX2, a gene known to be methylated in liver cancer, DMR was identified specifically in tumors compared to adjacent normal tissues and input samples (FIG. 1C and FIG. 7C). Next, by comparing methylomes of eight liver tumors to their corresponding adjacent non-tumor tissues, 11877 hypermethylated DMRs and 6976 hypomethylated DMRs were identified (FIG. 1D). To determine whether these DMRs identified in liver tumors showed concordance with DMRs of liver tumors from an independent source, the DNA methylation profiles of liver cancer were analyzed from TCGA, which were generated using 450K methylation microarrays. Despite the dramatic technical differences between MeDIP-Seq and 450K methylation arrays, hypomethylated and hypermethylated DMRs identified in liver tumors using gMeDIP-seq overlapped significantly with hypomethylated and hypermethylated DMRs identified using the TCGA liver cancer datasets, respectively (FIG. 1E and Table 1). In contrast, concordance between hypermethylated DMRs identified using eight liver cancer samples and hypomethylated DMRs using TCGA datasets and vice versa was not significant. Therefore, these results demonstrate the developed of a simple and reliable MeDIP-seq procedures for analysis of DNA methylomes using low amounts of genomic DNA.

TABLE 1
Patient information including cancer type, sex and
age of all 87 cfDNA samples used in this study

| | Training cohort | Validation cohort |
|---|---|---|
| | (N = 46) | (N = 41) |
| Sex and age | | |
| Male | 30 | 27 |
| Female | 16 | 14 |
| Age at diagnosis/recruitment | | |
| Young (<30 years) | 6 | 2 |
| Middle age (30~60 years) | 19 | 16 |
| Old age (>60 years) | 21 | 23 |
| Brain cancer | 20 | 13 |
| IDH WT | 10 | 7 |
| IDH mutant | 10 | 6 |
| Liver cancer | 16 | 18 |
| early stage | 5 | 7 |
| later stage | 11 | 11 |
| Controls | 10 | 10 |

Develop Single-Stranded (Ss)-cfMeDIP-Seq Method for Analyzing cfDNA Methylomes

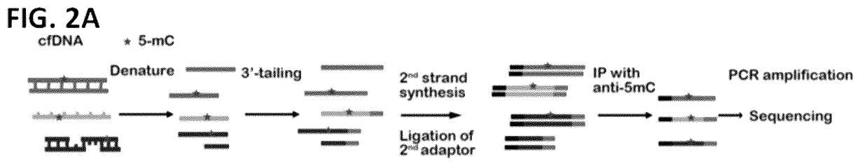

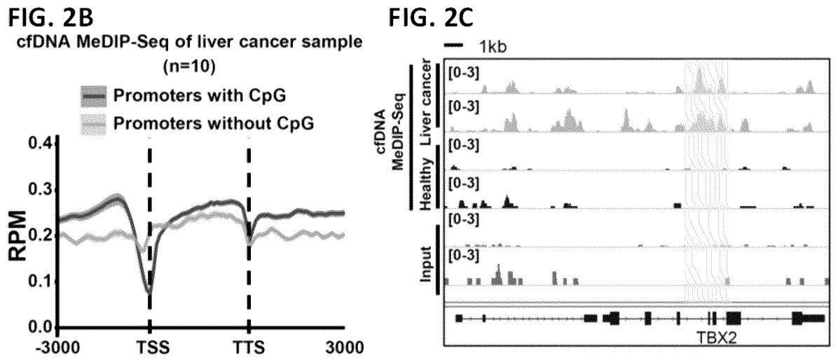

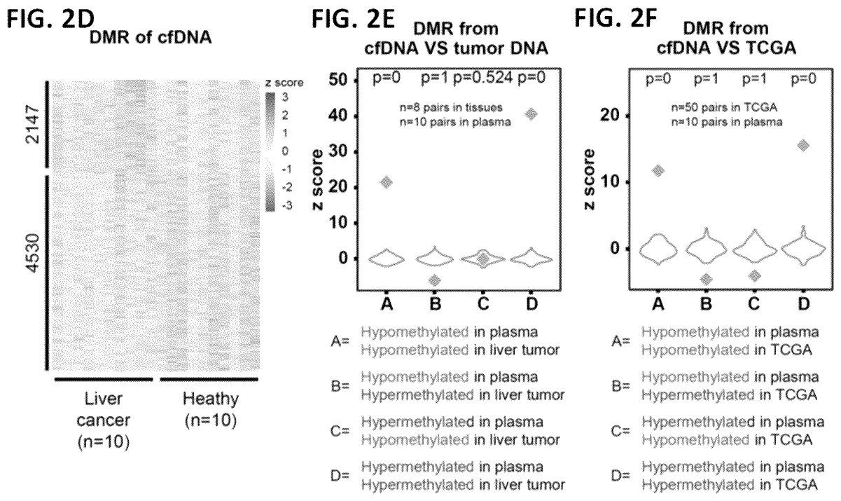

There is a tremendous interest in analyzing cfDNA methylomes for tumor detection. Compared to large dsDNA isolated from tumor samples, plasma cfDNAs are a mixture of dsDNA and ssDNA with fragment sizes about 160-170 nucleotides. Furthermore, some of these DNA are damaged. Therefore, it is impossible to apply the Tn5 based procedures outlined above to analyze methylomes of cfDNAs. A new MeDIP-Seq procedure termed single stranded (ss)-cfMeDIP-Seq was developed to analyze cfDNA methylomes. All DNAs including ssDNA, dsDNA and damaged DNA were utilized for methylome analysis (FIG. 2A). Briefly, after denaturing cfDNA into ssDNA, an adaptor was ligated to the 3′ end of cfDNA using an ssDNA ligase followed by converting ssDNA into dsDNA by a DNA polymerase. After the ligation of the second adaptor, a small fraction of DNA (10%) was used as input, and the remaining DNA was denatured again and subjected to immunoprecipitation using antibodies against 5-mC. The immunoprecipitated DNA as well as the input DNA were then amplified by PCR for library preparation and sequencing (FIG. 2A). This method can analyze DNA methylation on either Watson or Crick strands.

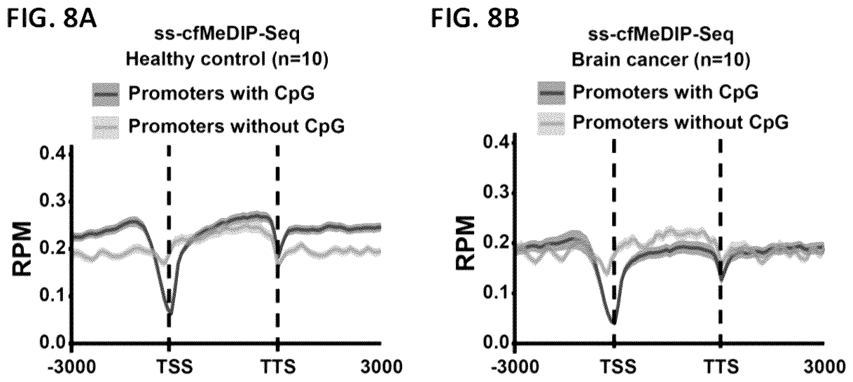

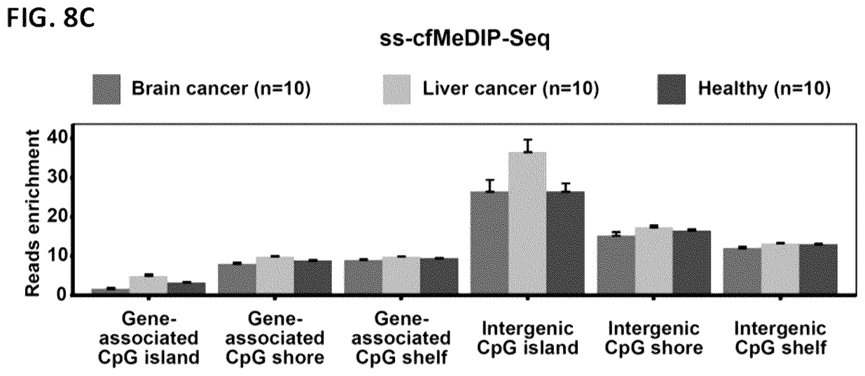

Using this method, high quality ss-cfMeDIP-seq datasets were produced from three groups of samples, controls (healthy) and those with liver or brain cancer. ss-cfMeDIP-seq signals were depleted at the promoter regions of genes with CpG islands compared to those without CpG islands for all the three groups (FIG. 2B, FIG. 8A, and FIG. 8B). Furthermore, ss-cfMeDIP-seq signals were enriched at intergenic CpG islands compared to other methylated regions such as gene-associated CpG islands (FIG. 8C). These distributions of cfDNA methylation are consistent with the general distributions of DNA methylation in cells, validating the reliability of the procedures for analysis of cfDNA methylomes.

To determine whether cfDNA methylomes could detect DNA methylation from tumor cells, methylomes of cfDNAs from patients with liver cancer were compared to healthy controls. Inspection of ss-cfMeDIP-seq signals at the TBX2 gene loci identified a hypermethylated region specific for liver cancer patients (FIG. 2C), suggesting these hypermethylated fragments are likely from liver tumor cells, but not from normal cells (FIG. 7A). To systematically test this idea, cfDNA methylomes from 10 liver cancer patients were compared to those of 10 control samples with similar age and sex distributions (Table 2), and 2147 hypermethylated DMRs and 4530 hypomethylated DMRs were identified (FIG. 2D).

TABLE 2
Patient information of 10 liver samples and 10 controls
to identify cfDNA DMRs described in FIG. 2A, FIG.
2B, FIG. 2C, FIG. 2D, FIG, 2E, and FIG. 2F

| | Liver Cancer Cases | Controls |
|---|---|---|
| | (N = 10) | (N = 10) |
| Sex | | |
| Male | 7 | 7 |
| Female | 3 | 3 |
| Unknown | 0 | 0 |
| Age at diagnosis/recruitment | | |
| Young (<30 years) | 0 | 1 |
| Middle age (30~60 years) | 4 | 3 |
| Old age (>60 years) | 6 | 6 |
| Unknown | 0 | 0 |

It was then determined whether these cfDNA DMRs overlapped with DMRs identified in liver tumor samples using gMeDIP-Seq and DMRs from TCGA datasets obtained using 450K DNA methylation arrays. Both hypermethylated and hypomethylated cfDNA DMRs exhibited significant overlap with hypermethylated and hypomethylated DMRs from liver tumor DNA analyzed by either gMeDIP-Seq (FIG. 2E) or from the TCGA datasets (FIG. 2F). In contrast, there were no significant overlaps between hypomethylated cfDNA DMRs and hypermethylated genomic DNA DMRs from liver tumors (FIG. 2E) or the TCGA datasets (FIG. 2F) or vice versa. Together, these results show that cfDNA methylomes generated by ss-cfMeDIP-Seq method most likely reflect DNA methylation changes in liver cancer cells.

Identification of Cancer Types Using cfDNA Methylomes and Deep Neural Networks

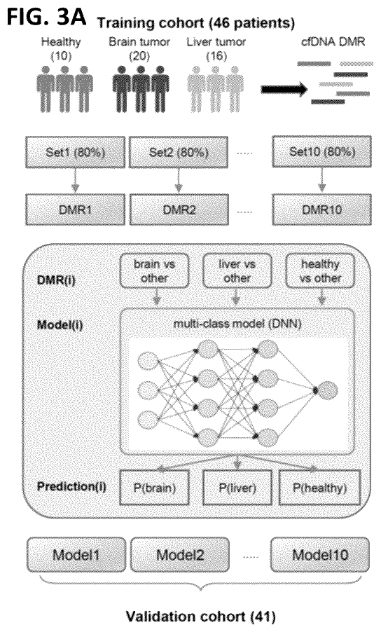

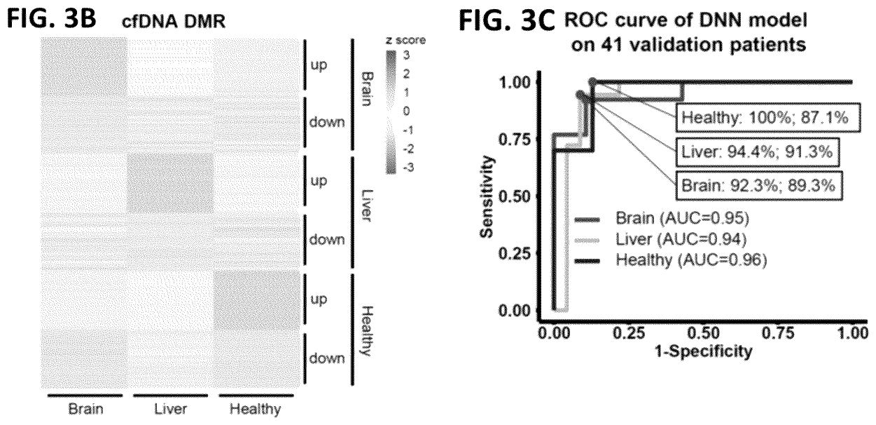

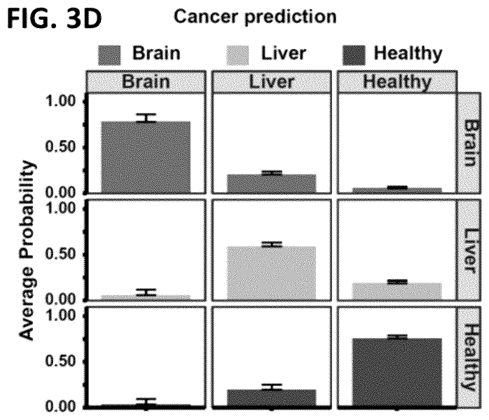

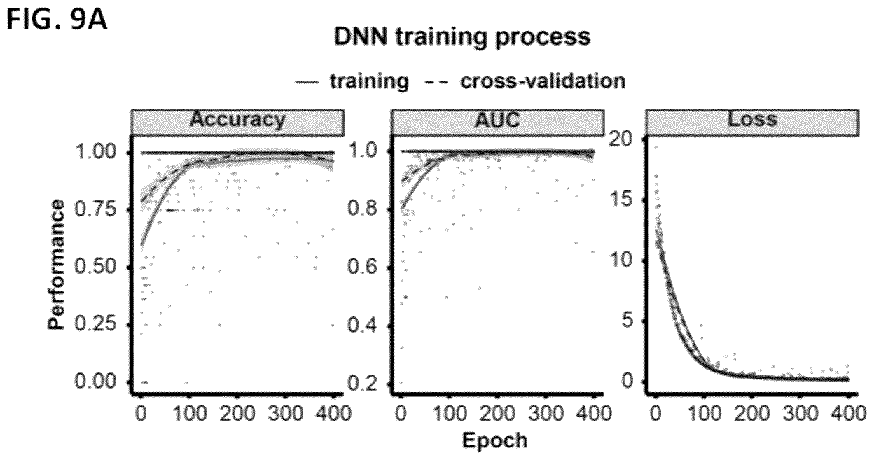

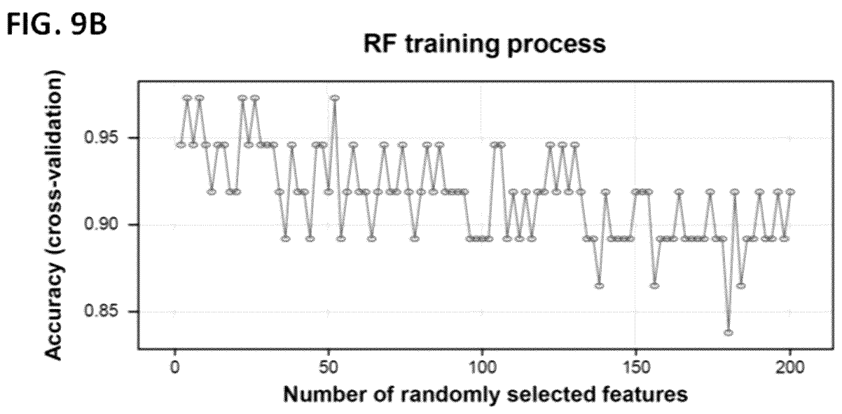

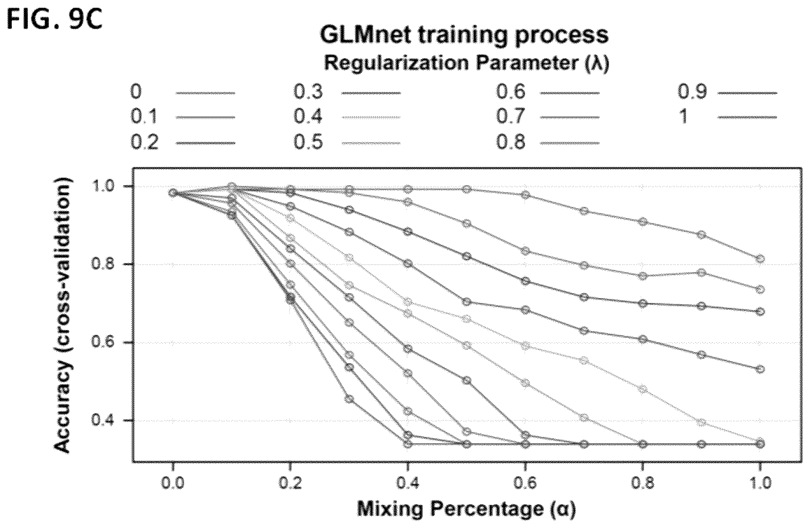

To determine whether ss-cfMeDIP-Seq procedures could be used for tumor prediction, cfDNA methylomes were analyzed from three groups of plasma samples: patients with liver (34 samples) or brain cancer (33 samples) and controls (20 samples) (Table 1). 20 samples shown in FIG. 2A were also included in these 87 samples. Of the 87 datasets generated, 46 datasets including 16 liver cancer and 20 brain cancer samples, and 10 controls were randomly selected from samples with more than six million unique reads and used as the training cohort. To reduce the influence of diversity of individual samples on the training models, 80% of the training cohort was randomly sampled 10 times in a balanced way (control, brain and liver cancer), and identified cfDNA DMRs specific for each sample group in a one-versus-other way and selected the top 100 hyper- and 100 hypo-DMRs for each sample group (FIG. 3A and FIG. 3B). In total, top 600 DMRs were selected from the three sample groups for each of the 10 rounds of training using a Deep Neural Network (DNN) model and 10 different DNN models were built based on the training cohort (FIG. 3A, FIG. 3B, and FIG. 9A). Each of 10 models were used to predict status in the remaining 41 samples in the validation cohorts (13 brain cancer patients, 18 liver cancer patients and 10 controls). Finally, the average probability of 10 predictions was reported as the final prediction probability. All 41 samples in the validation cohort were successfully identified with AUROC of 0.95, 0.94, and 0.96 for the brain cancer patients, liver cancer patients and controls, respectively. Moreover, the average probabilities of identifying brain cancer, liver cancer and control groups were 0.78, 0.59 and 0.76, respectively (FIG. 3D). Together, these studies indicate that cfDNA methylomes detected by the ss-cfMeDIP-seq can be used for tumor detection of liver and brain cancer with high confidence.

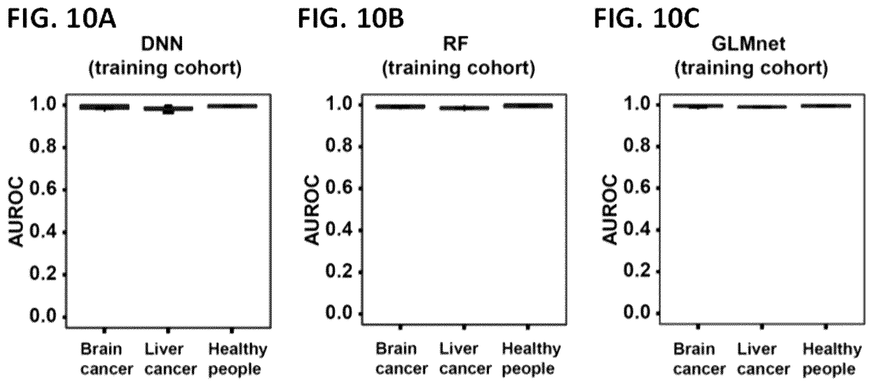

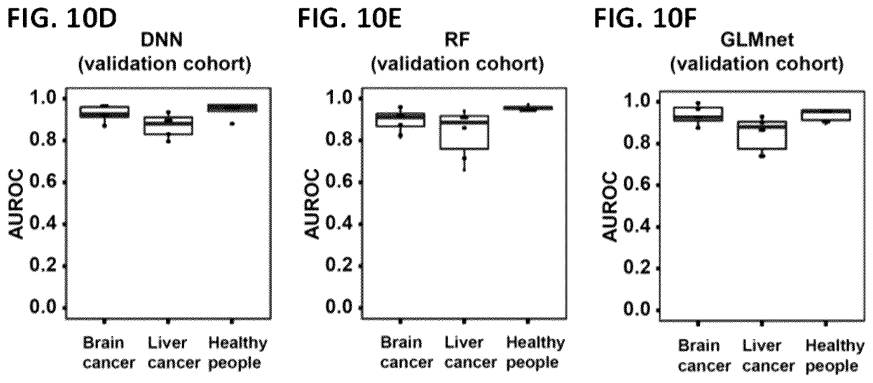

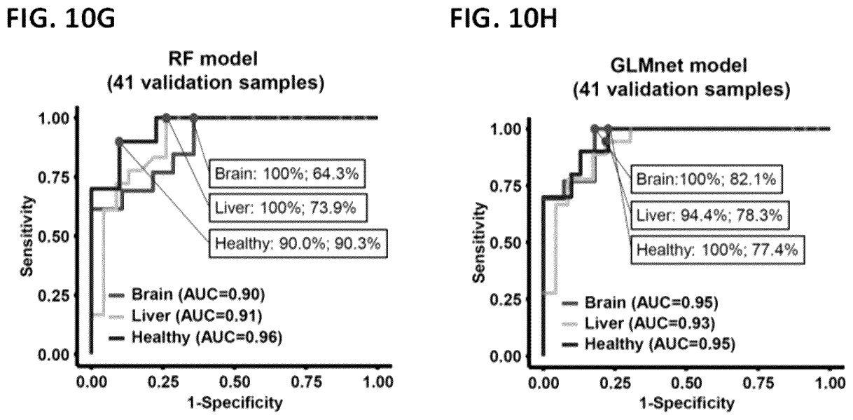

To analyze the cfDNA methylome further, training models were generated using two additional machine learning methods, Random Forest (RF), a non-linear classification method, and GLMnet, a generalized linear classification method (FIG. 9B and FIG. 9C) using the same 46-sample training cohort and then the status of the 41 samples in the validation cohort was predicted. All three models, RF and GLMnet and DNN, showed a high AUROC value >0.98 on the training cohorts (FIG. 10A, FIG. 10B, and FIG. 10C). Furthermore, both RF and GLMnet models also predicted brain and liver cancer in the validation cohort, with a slightly reduced performance compared to the DNN model (FIG. 10D, FIG. 10E, FIG. 10F, FIG. 10G, and FIG. 10H). Taken together, these results indicate that all three machine learning methods could be used to analyze ss-cfMeDIP-Seq datasets for cancer classification, with the best performance from the DNN model.

Differentiation of Glioma Subtypes by cfDNA Methylomes

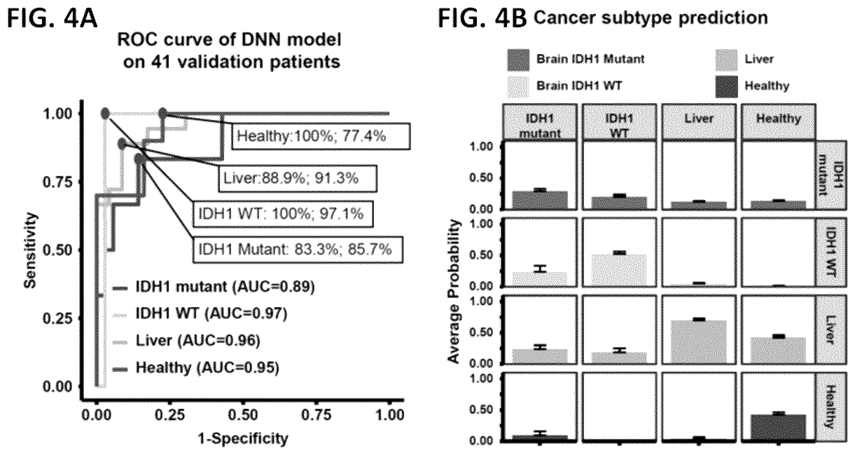

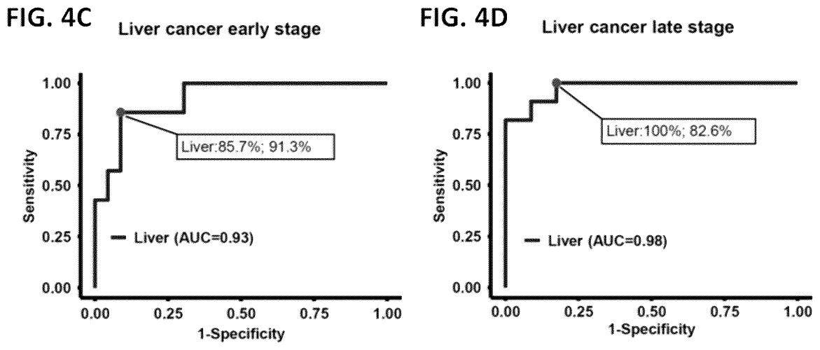

It was determined whether cfDNA methylome analysis can be used to differentiate the subtypes of brain tumors. Of 20 cfDNA samples from brain tumor patients in the training cohort, 10 samples were from patients with IDH mutations and 10 with IDH wild type tumors. Therefore, the 46 samples of the training cohort were separated into four groups (16 liver cancer, 10 gliomas with IDH mutation, 10 gliomas with IDH wild type and 10 controls), and followed the same procedures outlined above to train the DNN models. Briefly, after randomly sampling 80% of the training cohort in a balanced way for each sample group, the top 800 DMRs were selected with 200 from each group from the training cohort. In this way, 10 DNN models were built based on cfDNA methylomes of these 46 samples in the training cohort. Then, each of the 10 DNN models was applied to predict the status of the 41 samples in the validation cohort (18 liver cancers, 6 gliomas with IDH mutation, 7 IDH wild type gliomas, and 10 controls) 10 times. Finally, the average probabilities of the 10 predictions of each sample were calculated. As shown in FIG. 4A, the validation samples could be separated successfully into four groups with AUROC of 0.89, 0.97, 0.96, and 0.95 for subjects with IDH mutant gliomas, IDH wild type gliomas, liver tumors, and controls, respectively. The average probabilities of IDH mutation gliomas, IDH wild type gliomas, liver cancer, and control groups were 0.29, 0.52, 0.70, and 0.43, respectively (FIG. 4B). Together, these studies indicate that DNA methylomes generated by ss-cfMeDIP-Seq can accurately identify glioma subtypes.

Detection of Early-Stage Liver Cancer by cfDNA Methylomes Generated by Ss-cfMeDIP-Seq

Finally, it was determined whether cfDNA methylomes could be used to accurately detect early-stage liver cancer. Of the 18 liver cancer patients in the validation cohort, seven were classified as stage “A” (early stage), with the remaining 11 stage “B” or “C” (late-stage) liver tumors based on histology analysis. The capability of the DNN model was evaluated for detection of these two groups of liver cancer samples independently. As shown in FIG. 4C and FIG. 4D, the DNN model could identify seven early stage and 11 late-stage liver cancer samples with high accuracy and AUROC values of 0.93 and 0.98 for early and late-stage liver cancer, respectively. These results indicate that cfDNA methylomes generated by ss-cfMeDIP-Seq can also detect early-stage liver tumors.

### Evaluation of the Sensitivity of the Ss-cfMeDIP-Seq Method

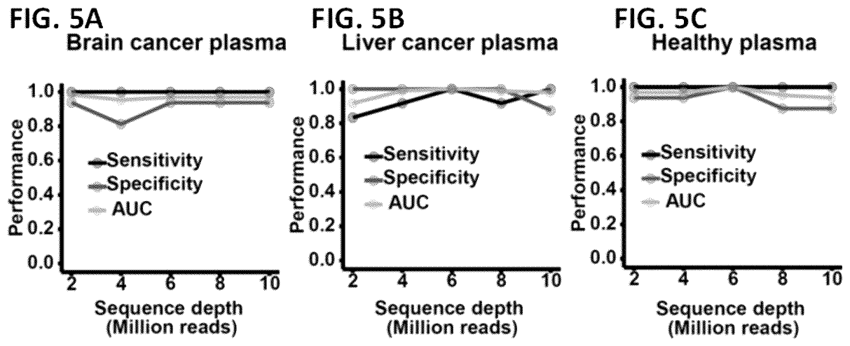

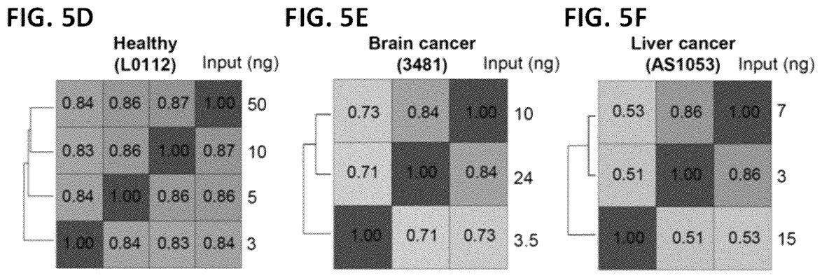

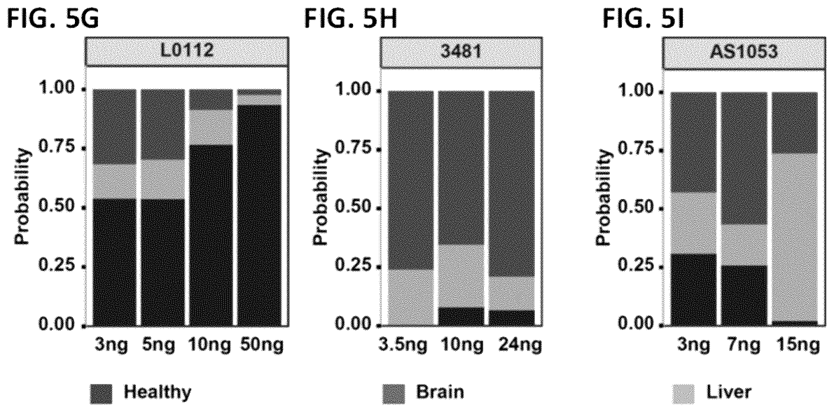

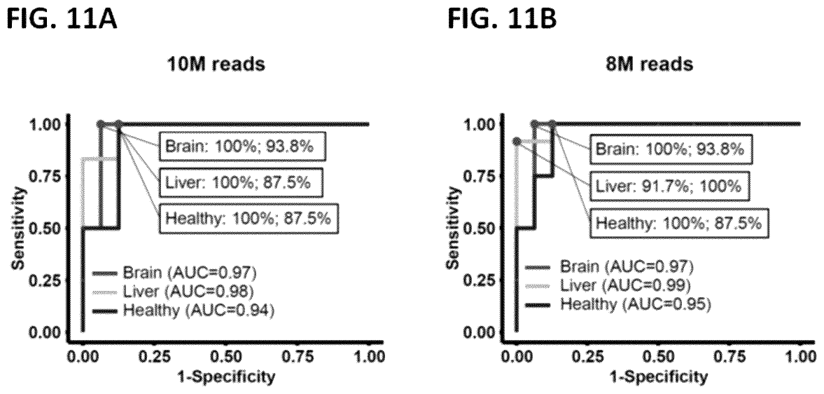

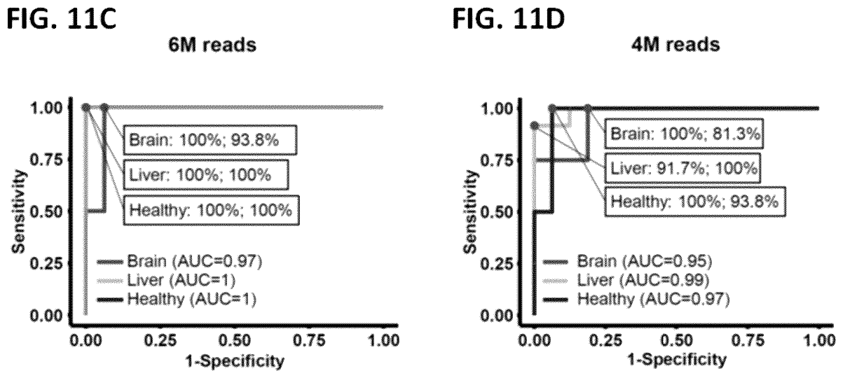

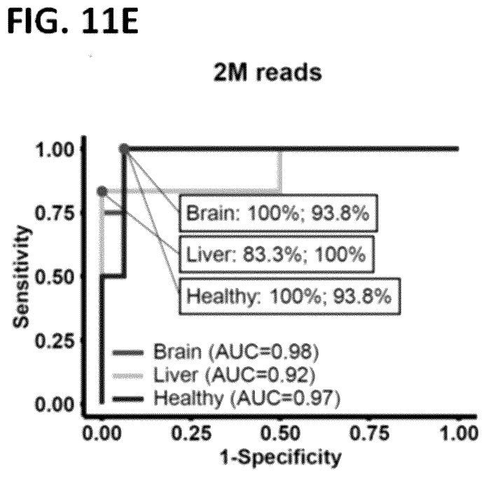

Because the cost of sequencing likely represents a major component of total costs for tumor detection using ss-cfMeDIP-Seq, the influence of sequence depth on prediction outcomes was evaluated. Briefly, 20 ss-cfMeDIP-Seq datasets were selected including 12 from liver cancer, four from brain cancer, and four from controls, each with more than 10 million unique reads, from the 41-sample validation cohort. 10, 8, 6, 4, and 2 million unique reads were then randomly selected from each of the 20 samples and generated datasets with different sequence depth. Next, the DNN models trained in FIG. 3A were applied to evaluate the prediction of these ss-cfMeDIP-Seq samples with different amount of sequence reads. The sequence depth of the validation samples did not, in general, affect the overall prediction outcome (FIG. 5A, FIG. 5B, FIG. 5C, FIG. 11A, FIG. 11B, FIG. 11C, FIG. 11D, and FIG. 11E). However, when the number of unique reads was below 6 million, there was a trade-off between specificity and sensitivity. For example, in the validation cohort containing 4 million reads, the sensitivity and specificity of the ROC curve for brain tumor samples were 0.81 and 0.95, respectively (FIG. 5A). With the same samples but with 2 million reads each, the sensitivity and specificity for liver cancer were 0.83 and 0.92, respectively (FIG. 5B). In contrast, in the validation cohorts with unique sequence reads over 6 million, the average sensitivity is above 0.97 and the average specificity is above 0.92 for each sample group.

The amount of cfDNA in plasma differs from sample to sample, with early-stage tumors in general releasing less cfDNA into blood than late-stage tumors. Therefore, the amount of cfDNA needed for the generation of high quality ss-cfMeDIP-Seq datasets for tumor prediction was tested. Briefly, three cfDNA samples were randomly chosen, with each from controls and individuals with liver and brain tumors, respectively. ss-cfMeDIP-Seq datasets were generated using different amounts of DNA isolated from each of the sample. For instance, four ss-cfMeDIP-Seq datasets were generated using 3, 5, 10, and 50 ng cfDNA from the control cfDNA, which was equivalent to 18, 30, 60, and 300 μl of plasma (FIG. 5D). The ss-cfMeDIP-Seq signals generated from these four different amounts of cfDNA were highly correlated (FIG. 5D). Three datasets were generated using three different amounts of cfDNA from one brain cancer sample and one liver cancer sample (FIG. 5E and FIG. 5F). High correlations were also observed among the three ss-cfMeDIP-Seq datasets generated using three different amounts of cfDNA of the brain cancer sample (FIG. 5E). However, the correlations between the ss-cfMeDIP-Seq dataset generated using 15 ng cfDNA of the liver cancer sample and those using lower amounts of cfDNA (3 and 7 ng) were poor (FIG. 5F), likely due to a high percentage of PCR duplications (about 70%) in the datasets generated using lower amount of input samples.

Next, the DNN model trained in FIG. 3A was applied to predict ss-cfMeDIP-Seq samples from different amounts of input DNA. The model could reliably predict brain cancer and healthy control samples with different amount of cfDNA (FIG. 5G and FIG. 5H). However, liver cancer could be predicted in the dataset generated using 15 ng cfDNA, but not with 3 or 7 ng cfDNA (FIG. 51). These results indicate that the amount of cfDNA isolated from different subjects varies, with higher input cfDNA generally yielding better quality of ss-cfMeDIP-Seq datasets. Furthermore, these studies indicate that ss-cfMeDIP-Seq datasets can be produced from as little as 20 μl plasma. Of note, cfDNA from 200 to 300 μl of plasma were used to generate ss-cfMeDIP-Seq datasets for almost all samples used in this study.

cfDNA DMRs of Liver and Brain Tumor Samples are Associated with Gene Expression in Tumor Samples and Patient Survival

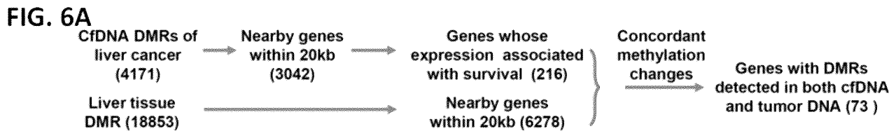

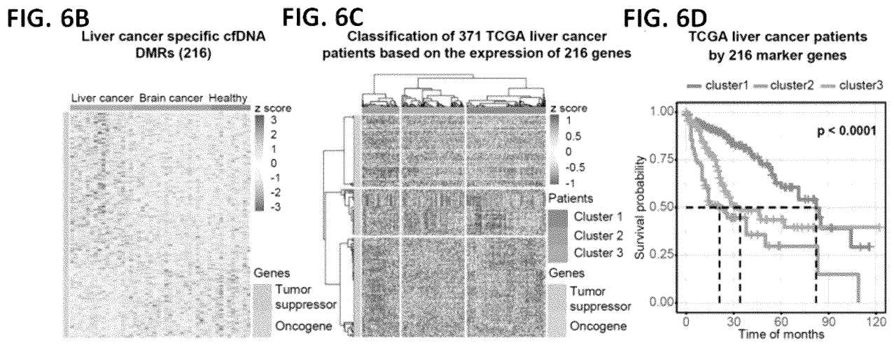

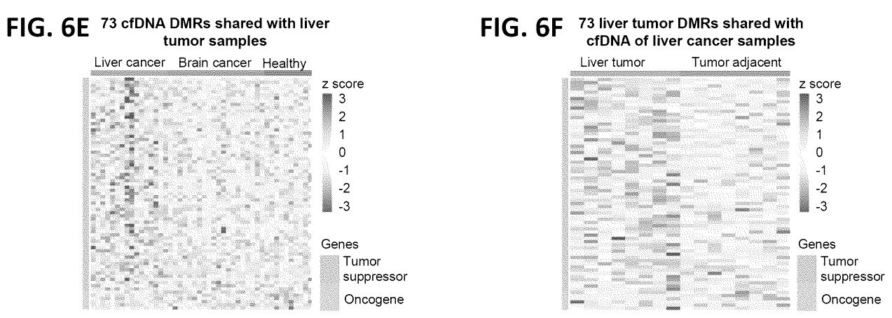

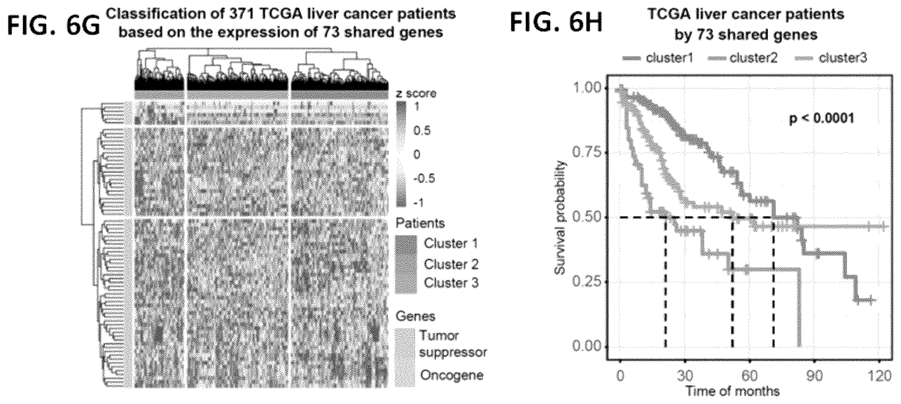

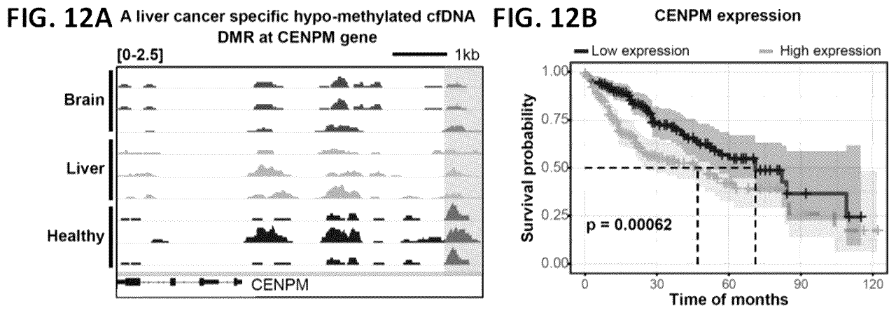

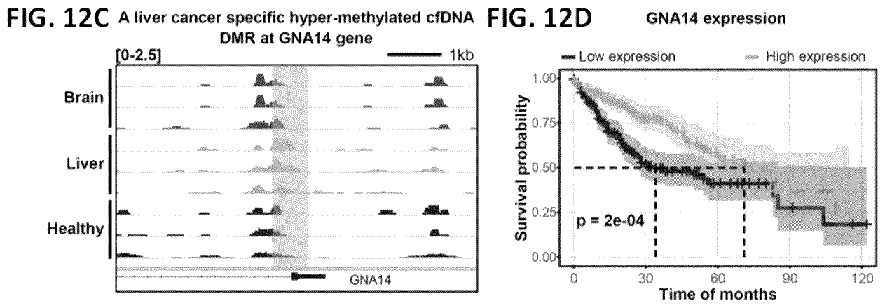

Promoter and enhancer DNA methylation is associated with gene transcription. To understand the relationship between cfDNA DMRs and gene expression in tumors, each of the 4171 cfDNA DMRs specific for liver cancer were annotated to their closest genes and 3042 genes were identified whose promoters were within 20 Kb of at least one of the 4171 cfDNA DMRs. Then, RNA-seq and clinical datasets of liver cancer from TCGA were downloaded and it was determined whether the expression of each of the 3042 genes is associated with patient survival (FIG. 6A). For instance, a hypomethylated region specific for liver cancer was identified at the CENPM gene locus compared to cfDNA from controls and patients with brain tumors (FIG. 12A). Furthermore, the high expression of CENPM in liver cancer tissue was significantly associated with poorer survival than in those with lower expression (FIG. 12B). In contrast, a hypermethylated region at the GNA14 gene locus was identified on cfDNA samples from liver cancer patients. Furthermore, high expression of GNA14 in liver tumor tissues was associated with better survival (FIG. 12C and FIG. 12D). Through the analysis, of the 3042 genes with at least one nearby cfDNA DMR specific for liver cancer, the expressions of 216 genes were associated with patient survival, suggesting that these genes, potentially regulated by DNA methylation, can be as biomarkers for liver cancer and are likely associated with tumorigenesis of liver cancer (FIG. 6B). These 216 genes were classified into “tumor suppressor genes” if their nearby cfDNA DMRs were hyper-methylated and patients with low expression of the gene showed poorer survival (p<0.05) than patients with high expression (e.g., GNA14). Conversely, if a gene has a hypo-methylated cfDNA DMR nearby and patients with high expression of the gene showed significantly poorer survival than those with low expression (p<0.05), the gene is referred to as an “oncogene” (e.g., CENPM).

Next, it was determined whether the expression of these 216 genes could be used to cluster the 371 TCGA liver cancer patient samples using unsupervised clustering analysis. The 371 liver cancer samples could be separated into three clusters, with “Cluster 1” showing high expression of “tumor suppressor genes”, “Cluster 2” showing high expression in oncogenes, and “Cluster 3” being an intermediate state between Cluster 1 and Cluster 2 (FIG. 6C). Importantly, patients in these three clusters showed dramatically different survival times, with the median survival of patients in Cluster 1 and Cluster 2 of ˜80 and 25 months, respectively (FIG. 6D). These results indicate that a significant fraction of cfDNA DMRs specific for liver cancer likely reflects the changes in expression of nearby genes that may contribute to tumorigenesis. To further validate these functional cfDNA DMRs, 18853 DMRS were identified from liver tumors and were annotated to their closest genes within 20 Kb, resulting in 6278 genes with liver tumor DMRs. Of the 216 genes identified in FIG. 6A based on their proximity with cfDNA DMR and the association of their expression with patient survival, 73 genes showed concordant DNA methylation changes in liver tumor samples, with the majority of these DMRs being hypermethylated in liver cancer (FIG. 6E and FIG. 6F). Importantly, 371 TCGA liver cancer samples could also be separated into three clusters based on the expression of these 73 genes, with patients in “Cluster 1” showing better survival and patients in “Cluster 2” showing poorer survival (FIG. 6G and FIG. 6H).

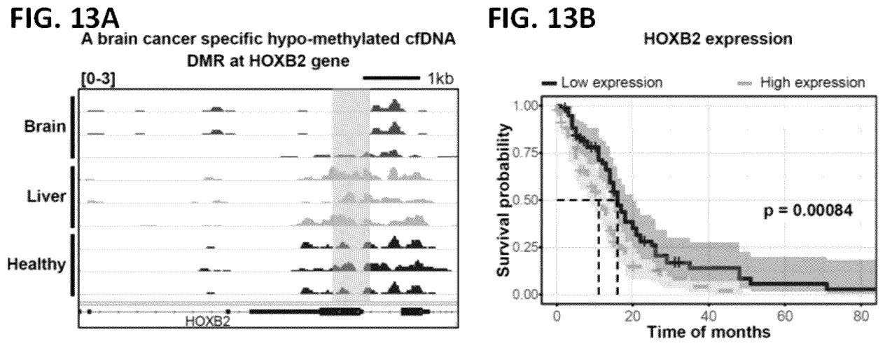

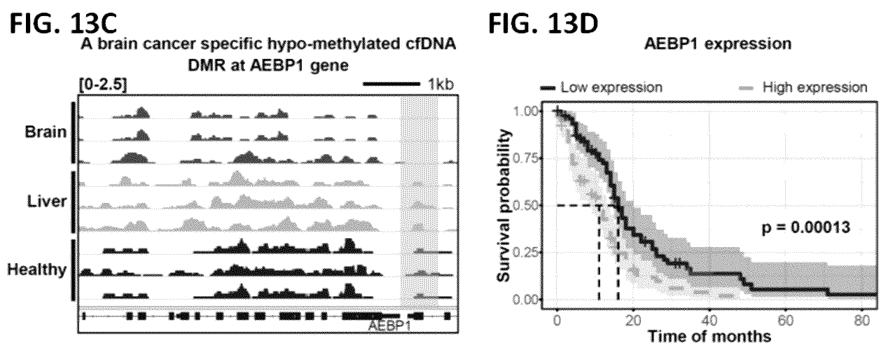

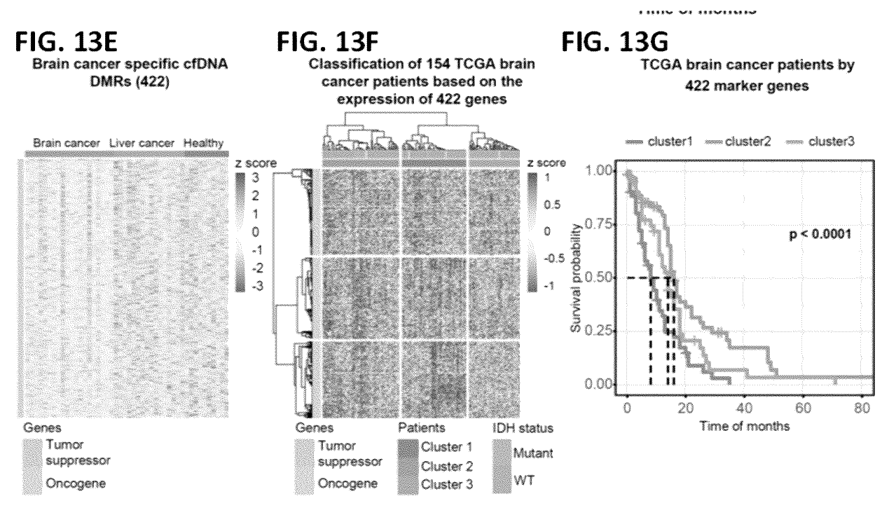

The same approach identified 422 genes with at least one brain tumor specific cfDNA DMR nearby whose expression in primary brain tumor tissues was associated with patient survival. For example, hypomethylated cfDNA DMRs were identified at both HOXB2 and AEBP1 gene loci (FIG. 13A and FIG. 13B), and brain tumors with high expression of these two genes showed poor survival (FIG. 13C and FIG. 13D). Interestingly, most of these brain tumor cfDNA DMRs are hypomethylated compared to samples from controls and liver cancer patients (FIG. 13E). Based on the expression of these 422 genes, 154 brain tumor samples from the TCGA database could also be separated into three clusters, with “Cluster 1” showing the highest expression of putative “oncogenes” and “Cluster 2” showing the lowest expression (FIG. 13F). Importantly, patients in Cluster 2 showed significantly better survival than patients in Cluster 1 (FIG. 13G). Patient samples with IDH mutations were enriched in “Cluster 2” (FIG. 13F), which is consistent with clinical results that brain tumor patients with IDH mutations have a favorable outcome compared to glioma patients with wild type IDH gliomas. Together, these studies indicate that cfDNA DMRs identified for both liver and brain tumor patients likely reflect changes in the expression of genes involved in tumorigenesis.

DNA cytosine methylation plays an important role in gene regulation, chromatin maintenance and genomic stability. Aberrant DNA methylation occurs in a variety of cancers. Therefore, DNA methylomes in cancer tissues have been used for tumor classification and detection. In this study, the gMeDIP-seq procedures were developed for analyzing methylomes of genomic DNA and ss-cfMeDIP-Seq was developed for plasma cell free DNA (cfDNA). These new and markedly simplified MeDIP-seq procedures greatly reduce the amount of DNA and time needed for the generation of MeDIP-seq datasets.

To optimize and simplify MeDIP-seq procedures for analyzing DNA methylomes of genomic DNA isolated from normal or tumor tissues, which in general are double-stranded large DNA fragments, pA-Tn5 loaded with one adaptor was utilized, which fragments and tags (aka “tagments’) dsDNA into small fragments. At the same time, oligos covalently linked to genomic DNA were introduced by pA-Tn5. In this way, the sonication step was not needed for shearing DNA into small fragments, which is the first step in previously published MeDIP-seq procedures. Furthermore, because of tagmentation, MeDIP-seq libraries can be generated through a simple PCR step without other complicated and inefficient steps such as primary ligation and/or T/A tailing described in published MeDIP-seq procedures. These modifications allow for the generation of high-quality MeDIP-seq libraries from 100 ng tumor DNA in less than two days.

Importantly, DMRs identified from six liver cancer samples using gMeDIP-Seq show significant concordance with DMRs of liver cancer samples based on DNA methylation analysis using 450K arrays. Together, these studies indicate that the gMeDIP-seq can be used to analyze DNA methylation of low amount genomic DNA (100 ng) including those isolated from cancer cells. One potential complication for the utilization of pA-Tn5 is over tagmentation of DNA fragments. However, relatively large DNA fragments (200-300 bp) were produced after tagmentation with pA-Tn5 when compared with Tn5 alone when the relative amount of genomic DNA and pA-Tn5 were controlled in the tagmentation reactions. Therefore, pA-Tn5 proteins described here may be suitable for the generation of next generation sequencing libraries without additional modifications.

In contrast to large dsDNA fragments for genomic DNA, most plasma cfDNAs are a mixture of dsDNA and ssDNA. Therefore, to optimize MeDIP-seq procedures for the generation of high quality cfDNA MeDIP-seq libraries, dsDNA was denatured to ssDNA first, and then the Swift 1S DNA library kit was used to mark the 3′ end of ssDNA molecules before immunoprecipitation with antibodies against 5mC. These modifications enabled the production of MeDIP-seq libraries using 5-20 ng ssDNA. While the amount of cfDNA varied from sample to sample, 5-20 ng single stranded cfDNA could be readily purified from 200-300 μl plasma for almost every sample, with as little as 30 μl plasma for some samples.

Furthermore, the procedures described herein can measure relative amount of methylation on both Watson and Crick strands. Using this method, cfDNA methylomes from 87 samples including controls and patients with liver or brain tumors were analyzed. 46 of 87 samples were chosen as the training cohort and DMRs for each sample group were identified, and these DMRs were used to train three different models based on Deep Neural Network (DNN), GLMnet model and Random Forest methods. Importantly, DMRs identified in each model could be used to predict tumor types of the remaining 41 samples in the validation cohort with the DNN model showing the best performance. Furthermore, the DNN models also differentiated IDH wild type and IDH mutant glioma samples as well as detected early-stage liver cancer. Importantly, these studies demonstrate that high-quality MeDIP-seq datasets could be generated from low amounts of cfDNA samples using the ss-cfMeDIP-Seq. It is generally accepted that much less cfDNA from brain tumor cells will be released into the blood than other tumor types.

Evidence is provided herein that cfDNA DMRs identified in this study likely reflect changes in DNA methylation in tumor tissues. First, cfDNA DMRs of liver cancer are highly concordant with DMRs identified from liver cancer samples. Importantly, cfDNA DMRs from liver and brain tumor samples could be used for the identification of genes whose expression in primary tumor tissue was associated with patient survival. For instance, 216 genes with at least one liver-specific cfDNA DMRs nearby and their expression in liver cancer samples is associated with patient survival. Importantly, the expression of these 216 genes in liver tumors from TCGA datasets can separate these samples into different clusters with different survival, strongly supporting the idea that these liver-cancer specific cfDNA DMRs likely reflect the changes in expression of genes involved in tumorigenesis. Indeed, a hypomethylated DMR was identified at the locus of CENPM, an oncogene in hepatocellular carcinoma (HCC) that promotes cell proliferation, migration and invasion in liver cancer. Furthermore, a hyper-methylated DMR was identified at the locus of GNA14, a tumor suppressor in HCC that is involved in regulation of the Rb pathway. Similar analysis of cfDNA DMRs from brain tumor patients also allowed the identification of genes whose expression in brain tumor tissues are also associated with patient survival. Together, these studies indicate that plasma cfDNA DMRs from liver and brain cancer patients can be used for prediction of tumor types as well as for the prediction of patient survival undergoing current treatment regimens.

### Biospecimens

Hepatocellular cancer patients' samples were from an IRB-approved, hospital-based prospective study conducted at Columbia University Irving Medical Center (CUIMC) that recruited HCC patients (>18 years older) from October 2008 to July 2014. Brain cancer patients' samples were collected as part of an IRB-approved protocol to collect, bank and distribute de-identified samples from brain tumor patients at CUIMC. Subjects without cancer were recruited from advertisements around CUIMC also with IRB approval. All subjects provided blood samples which were rapidly centrifuged to obtain plasma which was aliquoted and frozen at −80° C. until use. Basic epidemiologic variables were obtained by a structured questionnaire while clinical information on patients was obtained from medical records. Written informed consent was obtained from all participants and this research project was approved by the Columbia University Institutional Review Board.

### Protein, Antibody and Reagents

Purification of pA-Tn5 and pA-Tn5-oligo complex assembly used for analysis of methylation of tumor DNA were as described previously (Li, Z., et al., Efficient and strand-specific profiling of replicating chromatin with enrichment and sequencing of protein-associated nascent DNA in mammalian cells. Nat Protoc 16, 5739, 2021; Li, Z. M. et al., DNA polymerase alpha interacts with H3-H4 and facilitates the transfer of parental histones to lagging strands. Sci Adv 6, 2020). Antibodies against 5-mC (33D3) were purchased from Diagenode (C15200081). Mouse IgG used to bridge antibodies against 5-mC and pA-TN5 was purchased from Active Motif (53017), and tRNA was purchased from Sigma (R1753)

### Preparation of Genomic DNA

Genomic DNA was extracted from frozen tumor and adjacent tissues by standard proteinase K and RNase treatment followed by phenol and chloroform extraction. Tagmentation of genomic DNA was performed as previously described with minor modifications. In brief, 100 ng of DNA and 1.5 μl of pA-Tn5-AA complex were mixed in the Tagmentation buffer (5 mM TAPS-NaOH pH8.5, 5 mM MgCl2, 10% DMF), and were incubated in 37° C. with gentle shaking for 30 min. DNA was then purified by CHIP DNA clean kit (Zymo 11-379C), and oligo replacement and GAP repair followed the same procedures as described previously (Li, Z., et al., Efficient and strand-specific profiling of replicating chromatin with enrichment and sequencing of protein-associated nascent DNA in mammalian cells. Nat Protoc 16, 5739, 2021; Li, Z. M. et al., DNA polymerase alpha interacts with H3-H4 and facilitates the transfer of parental histones to lagging strands. Sci Adv 6, 2020). The processed DNA was then subjected to immunoprecipitation using antibodies against 5-mC described below.

### Preparation of Plasma Cell Free DNA

Plasma cell free DNA (cfDNA) was extracted from 0.5-1.5 ml of plasma using the Circulating Nucleic Acid Kit (Qiagen 55204) and AMPure XP beads. After purification, the ssDNA Assay kit (Invitrogen Q10212) was used to measure the concentrations of cfDNA. For each sample, 5-20 ng of cfDNA as determined by the kits was taken to the ligation step using a Swift 1S DNA library kit following the manufacturer's protocol.

### Methylated DNA Immunoprecipitation (MeDIP) and Library Preparation

The processed DNAs were diluted to 200 μl with the binding buffer (50 mM Tris pH 8, 350 mM NaCl, 0.05% Triton X-100, 1 mM EDTA), heated to 98° C. for 10 min, then cooled on ice immediately for 5 min. 5 μg tRNA (R1753 sigma) and 0.6 μg anti-5-mC monoclonal antibody 33D3 (C15200081) were added to the mixture and rotated at 4° C. for 1 hr. After addition of 1 μl of bridge antibody (Active Motif 53017) and 10 μl pre-washed Protein G beads (Invitrogen 10004D), the reaction mixtures were incubated at 4° C. for 16 hrs. After incubation, protein G beads were washed twice with the binding buffer, twice with wash buffer (50 mM Tris pH8, 140 mM NaCl, 0.05% Triton X-100, 1 mM EDTA) and twice with TE buffer. DNA on the beads was eluted twice at 65° C. for 15 min with 15 μl Elution buffer (10 mM Tris-HCl, pH8.0, 10 mM EDTA, 150 mM NaCl, 5 mM DTT, 1% SDS). Eluted DNAs were then combined and purified with CHIP DNA Clean & Concentrator (Zymo 11-379C). The purified DNAs were eluted in 20 μl low EDTA TE (Swift 90296). For the genomic DNAs, Illumina Nextera Dual Index primers were used for library amplification. Briefly, PCR reactions consisting of 20 μl eluted DNA, 1.5 μl 10 mM N7 primer, 1.5 μl 10 mM N5 primer, and 23 μl 2×PCR master mix (NEB 0541S) were assembled for library preparation.

For cfDNA samples, 1S Plus Set Indexing kits (Swift 16024) were used for sample indexing. Briefly, reaction mixtures consisting of 20 μl eluted DNA, 5 μl R1, 25 μl 2×PCR master were assembled in PCR tubes and used for PCR amplification (98° C. 1 min: 98° C. 10 sec, 63° C. 20 sec, 10-11 cycles: 72° C., 1 min). After PCR amplification, the reaction mixtures were mixed with 25 μl AMPure XP beads (Beckman A63880) for 5 min at room temperature. The supernatant was then transferred to a new tube with 25 μl of AMPure XP beads. After a 10-minute incubation at room temperature, the DNA on beads were washed twice with 200 μl 80% ethanol, and cluted with 14 μl low EDTA TE.

### MeDIP-Seq Data Analysis

MeDIP-Seq libraries were sequenced using a paired-end method on Illumina Nextseq 500/550 platforms. Adaptor sequences of all raw reads were removed by Cutadapt30 and reads <10 nt were removed. Reads passed through these cleanup steps were then mapped to the human reference genome (hg19) by Bowtie231. Duplicate reads were removed using Sambamba software32. Read coverage in a bin of 1 bp was calculated from filtered bam files by deepTools2 33 and then normalized with total number of filtered reads into reads per million (RPM).

Protein coding gene annotation was downloaded from GENCODE (v28) 34 and the CpG islands annotation was downloaded from UCSC Table Browser35. Protein coding genes were then classified into genes with and without CpG islands based on the overlap with their promoters ([−3 kb, 3 kb] surrounding TSS). Normalized reads density (RPM) of MeDIP-Seq was used to calculate from transcription start sites (TSS) to transcription termination sites (TTS) for each class of genes respectively by deepTools2 33.

Differentially Methylated Region (DMR) Identification for Genomic DNA and Plasma cfDNA

As described in Shen, S. Y. et al., Sensitive tumour detection and classification using plasma cell-free DNA methylomes. Nature 563, 579-583, 2018, a database of DNA methylation regulatory regions was built including FANTOM5 enhancers36, CpG islands35, CpG shores (2 kb flanking CpG islands) and CpG shelves (2 kb flanking outwards from a CpG shore). The datasets were collapsed to 1,086,958 windows of 200 bp as the candidate regions for DMR identification. To identify DMR of genomic DNA of liver tumor and adjacent regions, gMeDIP-seq datasets were compared from eight liver tumor samples to each corresponding adjacent normal region, and 11877 hypermethylated DMRs and 6976 hypomethylated DMRs were identified by DESeq238 with a cutoff of p<0.05 and |log 2 (fold change)|>1. For comparison, TCGA LIHC39 methylation datasets of Illumina 450K array were downloaded. Methylation level β values of 50 liver tumor samples were compared with their corresponding adjacent samples, and 10362 hypermethylated DMRs and 46969 hypomethylated DMRs were identified using the T-test with a cutoff of Bonferroni adjusted p<0.05.

To estimate the significance of overlaps between liver cancer DMRs identified in this study and TCGA liver tumor DMRs, 100 permutations were performed by bedtools 40 with the command “bedtools shuffle—incl regulation.bed”. 4432 concordantly hypermethylated and 3030 concordantly hypomethylated DMRs between the liver cancer DMRs found in this study and TCGA liver tumor samples using 450K methylation arrays were identified. The observed number of overlapping DMR (4432 and 3030) was compared with the null hypothesis distribution generated from corresponding 100 permutations and standard normalized to Z score (P value is calculated by the null distribution in a one-sided way). Both of the concordant hypermethylated and hypomethylated DMRs showed significantly (p=0 for 4432 hypermethylated DMRs and p=0 for 3030 hypomethylated DMRs, respectively) higher enrichment than random permutation distributions (FIG. 1E). However, discordant DMRs were not significant (p=0.68 for 17 DMRs and p=1.00 for 2 DMRs, respectively) (FIG. 1E).

To identify cfDNA DMRs, cfDNAs MeDIP-Seq datasets were compared from 10 liver cancer patients with those from 10 healthy controls using DESeq238 with a cutoff of p<0.05 and |log 2 (fold change)|>1. To evaluate whether these DMRs were also found in liver tumor DNA, the overlap between cfDNA DMRs was compared with DMRs identified using gMeDIP-seq in this study. cfDNA DMR identified from liver cancer samples was compared with DMRs identified using the TCGA datasets analyzed by 450K DNA methylation arrays. Similarly, discordantly methylated regions (hyper-methylated in one setting vs hypomethylated in another setting) between cfDNA DMR and DMRs on tumor DNAs from this study or from TCGA were also identified. Subsequently, the observed numbers of concordantly and discordantly overlapped DMRs were compared with the null distributions generated from the corresponding 100 random permutations. The observed numbers of overlapping DMRs were normalized to Z score by null distributions and p values were calculated in a one-sided way to evaluate the significance of overlap.

### Machine Learning Models

To detect and classify tumors by cfDNA methylomes, different machine learning models were built for tumor classification, including a deep learning model of Deep Neural Network (DNN), an ensemble learning model of Random Forest (RF) and a regularized regression model of Lasso and Elastic-Net Regularized Generalized Linear Model (GLMnet). Briefly, these 87 MeDIP-seq were separated into a training cohort and a validation cohort with a similar age and sex distribution. The training cohort consisted of 46 samples with more than 6 million unique sequenced reads (16 liver cancer, 20 brain cancer and 10 controls) and the validation cohort included 41 samples (18 liver cancer, 13 brain cancer, 10 controls). To reduce the influences of individual samples on the training model, 80% of the training cohort was sampled 10 times in a balanced way for each sample group (healthy, brain and liver), and specific DMRs for each sample group were identified in a one-versus-other way and the top 100 hyper- and 100 hypo-DMRs were selected for each group based on the Wald-statistic calculated by DEScq2. In each of 10 rounds, the top 600 DMRs were selected as features and the RPKM values were fed into the machine learning model for training. In total, 10 three-class machine learning models were built based on the training cohort. Each model was used to predict 41 samples in the validation cohort. Then, the average probability of each of the 10 subset models was calculated for each sample and used as the outcome of prediction. To evaluate the performance of machine learning models, the receiver operating characteristic (ROC) curve was plotted in sensitivity against (1-specificity) for each class, where sensitivity=(true positives/total positives) and specificity=(true negatives/total negatives). The area under ROC curve (AUROC) and its 95% confidence interval were also calculated for comparison by “PROC” package in R.

The Deep Neural Network (DNN) model is a deep learning architecture with multiple hidden layers of nodes between the input and output layers. Each node is a neuron with a linear or non-linear activation function and the whole DNN can handle complex non-linear relationships. Considering the relatively small sample size in this study, the model consisted of an input layer of 600 nodes, which were DMRs from each subset training cohort, an output layer of three nodes for the predicted probability of each of the three types, and two hidden layers with 64 and 32 nodes. To process the input signal, the activation function for the first hidden layer of 64 nodes was the rectified linear unit (ReLU); the second hidden layer of 32 nodes was the linear function; and the output layer was the softmax function for multinomial classification. The L2 penalty was set to 0.1 for regularization of the hidden layers to reduce the risk of overfitting. For the backprogation process when adjusting the weights for all the nodes, the default optimizer with a loss function of categorical cross entropy was used. During the training process, the batch size was set as default to 32 samples; the epoch number was set to 400 cycles based on learning curve graphs to make the model sufficiently trained and avoid overfitting (FIG. 9A). The validation data percentage was set to 0.1 to evaluate the model performance on each epoch. In this study, the DNN models were applied by “Keras” package in R.

Random forests combine decisions from multiple tree predictors to sample classification. Its generalization error depends on the strength of the individual trees in the forest and the correlations among these trees. To generate each tree and to avoid the correlated trees, only a subset of features is randomly selected to build the trees. In this study, the number of subset features for random selection was tuned over a grid of values by from 2 to (total features)/3 in increments of 2, and 1000 trees were generated in each round (FIG. 9B). Model training was performed using 10-fold cross-validation and applied by “caret” package in R.

GLMnet is a generalized linear model with the entire lasso or elastic-net regularization penalty. The elastic net penalty is controlled by a, bridging the gap between lasso regression (α=1) and ridge regression (α=0). The tuning parameter λ controls the overall strength of the penalty (λ=0 for no penalty and λ=1 for full penalty). In this study, α and λ were tuned over a grid of values to optimize the model from 0 to 1 in increments of 0.1, and the family function was set to “glmnet” for regression (FIG. 9C). The training was performed using 10-fold cross-validation and applied by “caret” package in R.

### Detection of Glioma Subtypes and the Stage of Liver Cancer

IDH mutant gliomas are present in >80% of World Health Organization grade II/III cases 22; patients with IDH mutant gliomas have a longer median survival time than IDH wild type glioma patients. Therefore, it was determined whether cfDNA methylomes generated by ss-cfMeDIP-Seq could be used to differentiate glioma subtypes. Briefly, the training cohort of 46 samples was separated into four groups (16 liver cancer, 10 IDH mutation, 10 IDH wild type and 10 controls) and the model was trained with four sample classes in a similar fashion as the model of three sample groups. The models were then used to predict samples of validation cohort.

The Barcelona Clinic Liver Cancer (BCLC) system classifies liver cancer into four stages. Stage “A” or early-stage tumors are from patients with asymptomatic tumors suitable for radical therapies, resection, transplantation or percutaneous treatments. Stage “B” or intermediate stage are from patients with asymptomatic multinodular HCC. Stage “C” or the advanced stage are from patients with symptomatic tumors and/or an invasive tumor pattern. Stage “D” is the end-stage of patients with extremely grim prognosis that should merely receive symptomatic treatment. To further evaluate the models' performance for detection of early-stage liver cancer, cfDNA samples were separated into early stage (stage A) and late stage (stage B and C) and interrogated these samples separately with the models.

### Effects of Sequence Depth of Ss-cfMeDIP-Seq Datasets on Tumor Predictions

To evaluate effects of the sequence depth of ss-cfMeDIP-Seq datasets on tumor detection, 20 samples were selected from the validation cohort, each with over 10 million unique reads. Samtools was used to randomly extract 8, 6, 4 and 2 million reads for each sample by command “samtools view -bhS-s depth/total.reads bam.file>out.file” and generated validation cohorts of 20 samples, with the sequencing reads from 10 to 2 million reads. The DNN models were then used to evaluate these in silico generated validation cohorts with different sequence depth.

Link cfDNA DMRs with the Expression of their Nearby Genes in Tumor Tissues and Patient Survival

After the identification of liver and brain tumor specific cfDNA DMRs by the Wald test statistic derived from DESeq, each DMR was annotated to their closest genes and genes were selected whose promoters are within 20 Kb of liver cancer-specific or brain cancer-specific DMRs. The following analysis was then performed on these nearby genes. The RNA expression in RSEM value (RNA-Seq by Expectation Maximization) and patient's clinical data was downloaded from TCGA-LIHC project (371 liver cancer patient samples) and TCGA-GBM project (154 brain cancer patients). Based on the median RNA expression of each of these nearby genes in patients, liver or brain cancer samples were separated into two groups, high and low expression. The cox proportional hazards model was performed for each of the nearby genes to evaluate hazard ratio on patients' survival. The genes whose expression in these cancer samples is associated with patient survival were chosen for further analysis.

### Data Availability

The cfDNA MeDIP-Seq data in this study are all deposited on Gene Expression Omnibus repository (GEO) under accession GSE213890. The R scripts and machine learning models are deposited on GitHub (github.com/clouds-drift/cfDNA_cancer_detection).

TABLE 3
Overlaps between liver cancer DMRs identified using gMeDIP-
Seq in this study with those derived from TCGA dataset
analyzed by 450K methylation arrays. Odds ratio =
4.5e+15, p-value < 2.2e−16 by Fisher's exact test.

| | Hypermethylated | Hypomethylated |
|---|---|---|
| | in liver tumor | in liver tumor |
| Hypermethylated | 4432 | 2 |
| in TCGA | | |
| Hypomethylated | 17 | 3030 |
| in TCGA | | |

TABLE 4
Overlaps between cfDNA DMRs from liver cancer samples
and tumor DMRs from the TCGA dataset. Odds ratio =
208.0, p-value < 2.2e−16 by Fisher's exact test.

| | Hypermethylated | Hypomethylated |
|---|---|---|
| | in TCGA | in TCGA |
| Hypermethylated | 76 | 11 |
| in plasma | | |
| Hypomethylated | 5 | 159 |
| in plasma | | |

TABLE 5
Overlaps between cfDNA DMRs from liver cancer and liver tumor
DMRs identified using ss-cfMeDIP-Seq in this study. Odds
ratio = 59.3, p-value < 2.2e−16 by Fisher's exact test.

| | Hypermethylated | Hypomethylated |
|---|---|---|
| | in liver tumor | in liver tumor |
| Hypermethylated | 305 | 22 |
| in plasma | | |
| Hypomethylated | 34 | 148 |
| in plasma | | |

### Example 2

Aberrant DNA methylation plays a critical role in tumorigenesis. While DNA methylation has been used for cancer detection and classification, DNA hemi-methylation, a novel epigenetic mark found at about 10% CpG dinucleotides, has not been analyzed extensively in cancer epigenomes. Here it is reported that methylated DNA immunoprecipitation and strand-specific (ss) sequencing (MeDIP-Seq) for genomic DNA (ssg-MeDIP-Seq) and plasma cell free (cf) DNA (sscf-MeDIP-Seq), which analyze both symmetrically methylated DNA regions as well as hemi-methylated regions of genomic DNA and plasma cfDNA, respectively. Differentially methylated regions (DMRs), which have been used previously for tumor detection, and differentially hemi-methylated regions (DHMRs) of liver tumor DNA or plasma cfDNA do not overlap, suggesting that DHMRs are novel biomarkers different from DMRs for tumor detection. Furthermore, using the sscf-MeDIP-Seq method, plasma cfDNA methylomes of 221 samples were analyzed from subjects with liver cancer and brain cancer and individuals without cancer (controls). Among them, 175 samples were chosen as the discovery cohort for the identification of DMRs and DHMRs specific in each subject group and for the training of machine learning models of multi-cancer detection (MCD) using DMRs, DHMRs and DMRs+DHMRs as inputs. These models were then used to predict the 46 samples in the validation cohort. Models trained with DMRs+DHMRs as inputs in general outperform models trained with DMRs or DHMRs alone, with AUROC being 0.971, 0.981, and 0.99 in predicting control, liver and brain cancer samples in the validation cohort, respectively, by the DMR+DHMR-trained models. Moreover, brain tumor subtypes can be accurately detected in the validation cohort. Together, these studies reveal a unique and robust method, which utilizes both cfDNA methylation and hemi-methylation as biomarkers, for multi-cancer detection.

Cancer is a major public health threat worldwide. While the cancer death rate has fallen continuously since the peak in 1991 in the United States, it is estimated that over 600,000 people died from cancer in 2021 in the United State alone. World-wide, almost 10 million people died from cancer in 2020, and the death rate increased in some developing countries in recent years. Therefore, there remains an urgent and unmet need to combat cancer. It has been shown that early tumor detection has potential to improve prognosis for cancer patients. For instance, it is estimated that the five-year survival rate for hepatocellular carcinoma (HCC) when diagnosed at early and localized stage is 34%, but drops to 3% when diagnosed at late stage with distant disease. Early cancer detection also contributed to the reduced cancer death rate in the United States in the last couple of decades. Therefore, it is critically important to develop assays for early cancer detection.

Recently, it has been shown that liquid biopsy offers several advantages for cancer early detection. For instance, liquid biopsy samples can be obtained non-invasively and in principle can overcome the challenges arising from tumor heterogeneity that confounds tissue biopsy procedures. Indeed, several methods have been developed to use plasma cell free (cf) DNA for tumor detection. Plasma cfDNA molecules are a mixture of extracellular DNA fragments released from apoptotic and/or necrotic cells or released via active secretion. While the majority of the plasma cfDNA comes from normal cells such as lymphocytes, cancer cells also release DNA fragments into circulation. Analysis of cancer related mutations in plasma cfDNA has been reported for early cancer detection. Due to limited mutations in cancer cells and the evolving nature of these mutations, a significant amount of test material (7.5-10 ml plasma) as well as sequence depth are required for specific mutation detection. In addition to genetic mutations, other cfDNA features including fragmentomics, and epigenetic features including nucleosome patterns and DNA methylation have also been analyzed for detection of a variety of cancer types. Among all these features analyzed, it is reported that whole genome DNA methylation is the best to detect a cancer signal compared to other features including single nucleotide variants paired with white blood cell background removal or cfDNA pan features including fragment length. However, it remains challenging to analyze plasma cfDNA methylomes using a small amount of plasma.

The vast majority of DNA methylation in mammalian cells occurs at CpG dinucleotides in a symmetric manner: the cytosines (C) in a CpG dinucleotide on both Watson strand and its complementary Crick strand are methylated. During DNA replication, hemi-methylated CpG dinucleotides, consisting of methylated CpGs on the parental strand and non-methylated CpGs on the complementary nascent strand are rapidly converted into symmetric and fully methylated CpGs to maintain DNA methylation patterns. While early studies indicate that failure of symmetrical methylation and intermediates of active demethylation in cancer genesis contributes to the generation of hemi-methylated DNA, it has been observed that about 10% of CpG dinucleotides in human embryonic stem cells are hemi-methylated which could be maintained during multiple cell divisions. Therefore, DNA hemi-methylation is a novel epigenetic mark. However, very few studies, have explored these hemi-methylated regions alone or in combination with symmetrically methylated CpGs for tumor detection and for tumorigenesis.

Currently, various methods have been used to analyze cfDNA methylomes for cancer detection. For instance, the CCGA (Circulating Cell-Free Genome Atlas) employed targeted bisulfite sequencing to analyze methylated regions of cfDNA in more than 50 cancer types. Because bisulfite treatment of DNA results in a marked loss of DNA, this method, on average, requires up to 80 ml plasma and over 100 million sequence reads per sample for tumor detection. Shen, S. Y. et al., (Nature 563, 579-583, (2018)) and Nassiri et al., (Nat Med 26, 1044-1047, (2020) developed a cell-free methylated DNA immunoprecipitation and high-throughput sequencing (cfMeDIP-seq) method based on double strand DNA ligation. This method requires cfDNA purified from 0.5-3.5 ml plasma samples. Because a large fraction of cfDNA molecules are single-stranded (ss) DNA fragments and damaged double-stranded DNA fragments, these DNA molecules were unlikely to be used in cfDNA methylome analysis. If all cfDNA molecules including ssDNA, dsDNA and damaged DNA were analyzed, this in principle would increase sensitivity and reduce the amount of plasma needed for analyzing cfDNA methylomes for tumor detection. Importantly, none of these studies, as designed, evaluated DNA methylation on Watson and Crick strand separately, a prerequisite for detecting DNA hemi-methylation.

Described herein are two new methylated DNA immunoprecipitation and strand-specific (ss) sequencing methods (MeDIP-Seq) for genomic DNA (ssg-MeDIP-Scq) and plasma cell free (cf) DNA (sscf-MeDIP-Seq) for analysis of methylomes of genomic DNA and cfDNA, respectively. The sscf-MeDIP-Seq method can analyze methylomes of all cfDNA molecules including ssDNA, dsDNA and damaged DNA. Importantly, both methods analyze symmetrically methylated and hemi-methylated regions. Through in-depth analysis of differentially methylated regions (DMRs) and differentially hemi-methylated regions (DHMRs) of liver tumor DNA and cfDNA samples, the vast majority of tumor DNA as well as cfDNA DMRs do not overlap DHMRs of the same samples, suggesting that DHMRs can serve as biomarkers independent of DMRs. Furthermore, cfDNA methylomes were analyzed from 221 samples from individuals without cancer (controls), and patients with brain or liver cancer. Reliable sscf-MeDIP-Seq datasets were produced with cfDNA isolated from 300-500 μl of plasma. Importantly, machine learning models using both DMRs and DHMRs as input features outperform models based on DMRs or DHMRs alone for tumor detection. Together, these findings reveal that the utilization of both cfDNA DMRs and DHMRs identified by the sscf-MeDIP-seq procedures as biomarkers is a highly sensitive and accurate approach for multi-cancer detection.

Develop Genomic Methylated DNA Immunoprecipitation with a Strand-Specific Sequencing Method (Ssg-MeDIP-Seq)

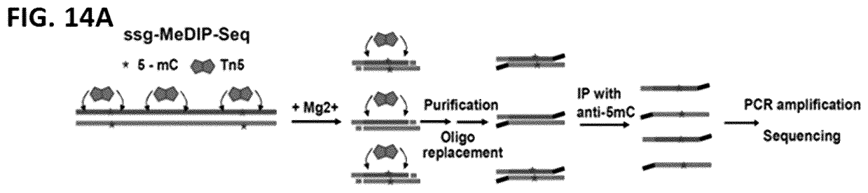

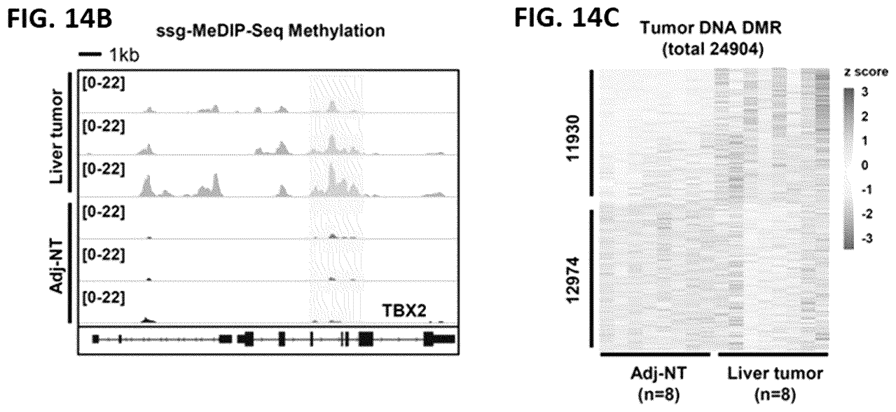

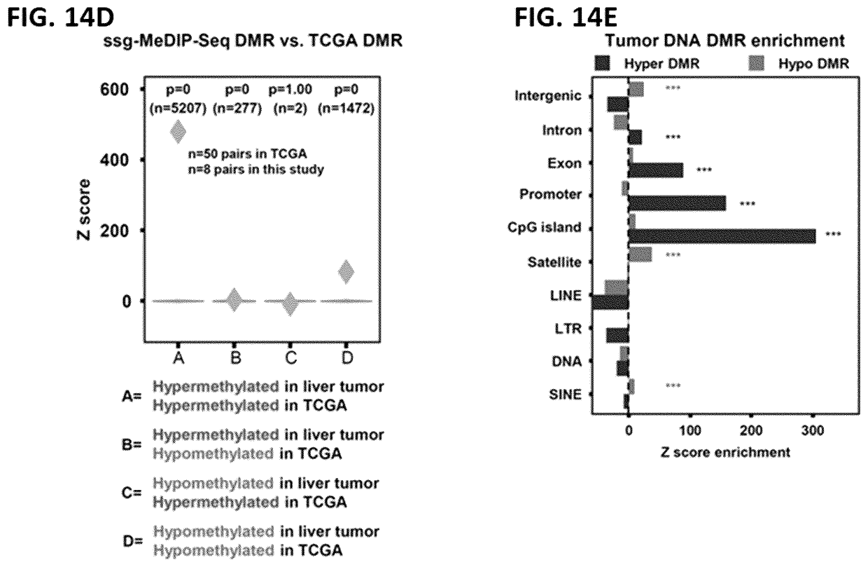

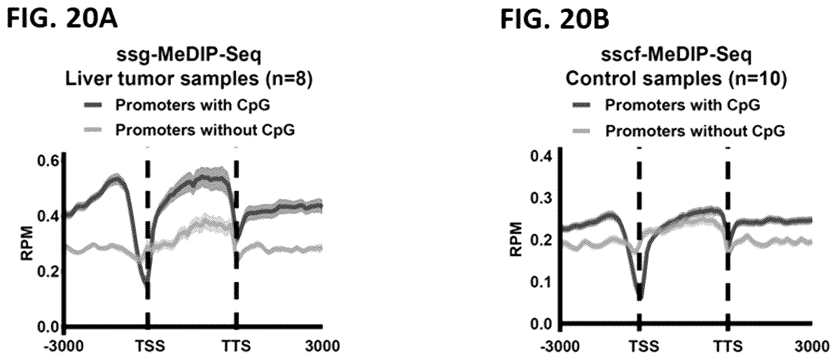

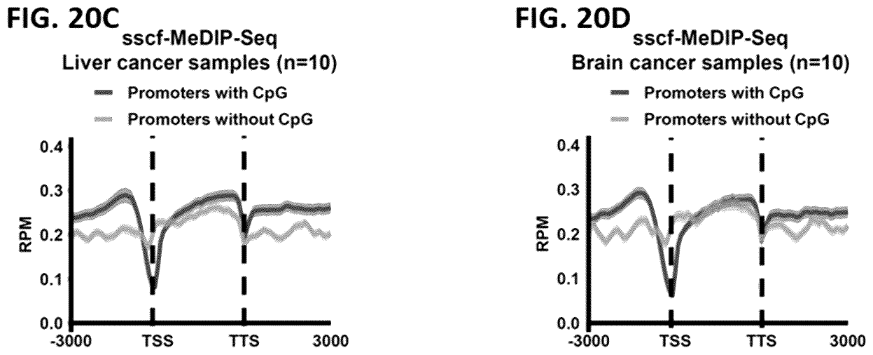

MeDIP-seq has been used to analyze DNA methylation (5-mC), and almost all published MeDIP-Seq procedures rely on sonication of genomic DNA into small fragments followed by immunoprecipitation with antibodies against methylated DNA. As Tn5 transposase has been used for genomic DNA fragmentation for the generation of libraries for next generation sequencing, it was assessed whether Tn5 can be used for fragmentation of genomic DNA before immunoprecipitation (FIG. 14A). Briefly, 100 ng of genomic DNA isolated from tissues was incubated with pA-Tn5 transposase, which fragments and inserts an adaptor into dsDNA in a sequence independent manner. As pA-Tn5 transposase covalently ligates one strand of the adaptor to the target DNA, a different adaptor was ligated at the 3′ end through the oligo-replacement step. In this way, DNA methylation patterns could be analyzed in a strand-specific manner, which in turn allows detection of both DNA methylation density as well as hemi-methylated regions. This method is termed ssg-MeDIP-Seq herein. Following the adaptor ligation, DNA fragments were denatured into single-stranded DNA (ssDNA) and methylated DNAs were immunoprecipitated using antibodies against 5-mC. The enriched methylated ssDNAs were amplified by PCR for library preparation and subsequent sequencing (FIG. 14A). Using this method, DNA methylation of 16 tissue samples were analyzed, eight isolated from liver tumors and eight from their corresponding adjacent non-tumor (Adj-NT) tissues. By inspection of MeDIP-seq signals at the gene locus of TBX2, a gene known to be methylated in liver cancer, a DMR was specifically identified in tumors compared to Adj-NT samples (FIG. 14B). Furthermore, ssg-MeDIP-seq signals were depleted at the promoters of genes with CpG islands compared to those without CpG islands (FIG. 20A), a pattern consistent with DNA methylation detected using other methods. Next, by comparing methylomes of eight liver tumors to their corresponding adjacent non-tumor tissues at 2,002,724 DNA methylation blocks defined recently, which cover 70% of CpG dinucleotides in the genome with each block consisting of at least four CpGs, 11,930 hypermethylated DMRs and 12,974 hypomethylated DMRs were identified (FIG. 14C). To determine whether these DMRs identified in liver tumors showed concordance with DMRs of liver tumors from an independent source, the DNA methylation profiles were analyzed of liver cancer from TCGA, which were generated using 450K CpGs methylation microarrays. Despite the dramatic technical differences between ssg-MeDIP-Seq and 450K methylation arrays, hypomethylated and hypermethylated DMRs identified in liver tumors using ssg-MeDIP-seq overlapped significantly with hypomethylated and hypermethylated DMRs identified using the TCGA liver cancer datasets, respectively (group “A” and “D” in FIG. 14D). In contrast, concordance between hypermethylated DMRs identified using ssg-MeDIP-Seq and hypomethylated DMRs using TCGA datasets and vice versa was not significant (group “B” and “C” in FIG. 14D). Finally, hyper-methylated DMRs were enriched at exons, promoters and CpG islands, whereas hypo-methylated DMRs were enriched at intergenic regions and SINEs (FIG. 14E). Taken together, these results indicate that ssg-MeDIP-seq is a simple and reliable procedure for analysis of DNA methylomes using low amounts of genomic DNA.

### Liver Tumor DNA DHMRs and DMRs are Independent Biomarkers

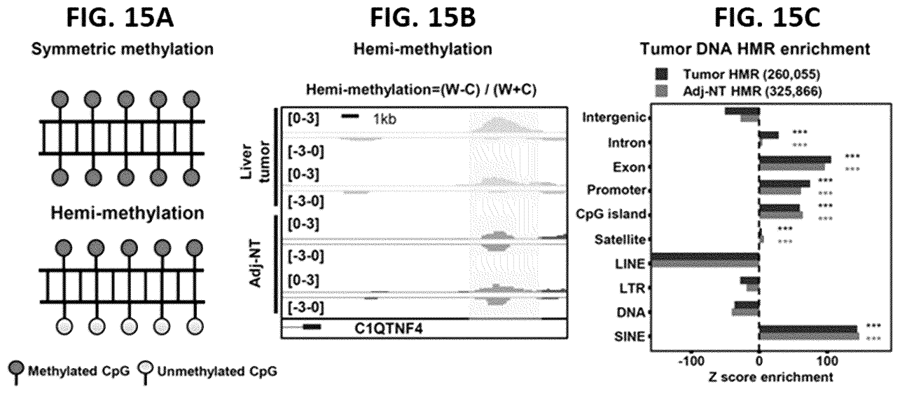

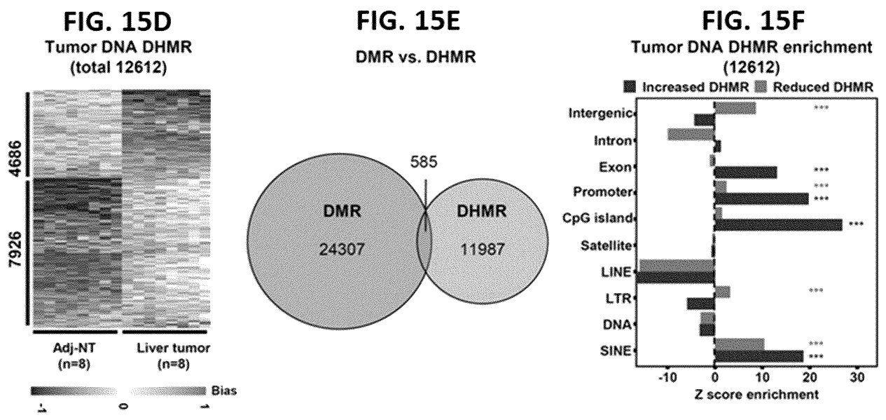

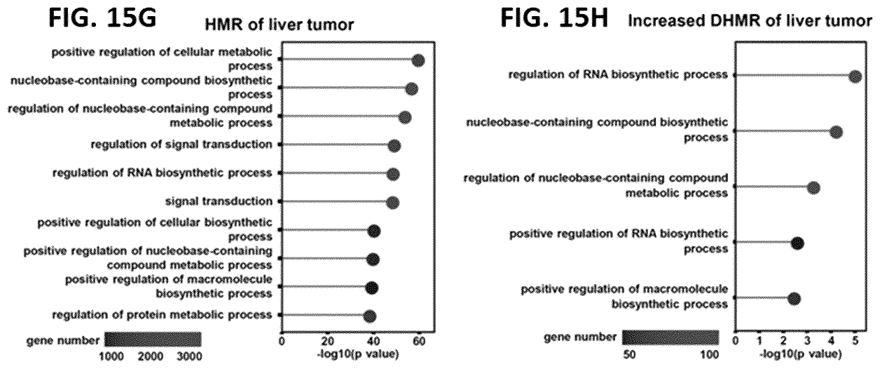

Recently, it has been shown about 10% of CpG dinucleotides are hemi-methylated (FIG. 15A), and these hemi-methylated regions are heritable. However, no studies have been performed to compare differentially hemi-methylated regions (DHMR) and differentially methylated regions in any given cancer type. Because the ssg-MeDIP-Seq method makes it possible to detect DNA methylation at Watson and Crick strands separately, hemi-methylated regions (HMRs) at 2,002,724 blocks were analyzed in 8 liver tumor samples and their matched Adj-NT controls. At the cutoff of hemi-methylation (Watson-Crick)/(Watson+Crick)>0.3 and p<0.01, 260,055 and 325,866 HMRs were identified in 8 liver tumor samples and their Adj-NT controls, respectively. The HMRs of both liver tumor and Adj-NT controls were enriched the most at genomic regions of SINEs, CpG islands, promoters and exons, and with a slight enrichment at satellites and introns (FIG. 15C). Furthermore, 12,612 DHMRs were identified in liver tumor DNA samples compared to their Adj-NT controls with the cutoff of p<0.01 and Δ|hemi-methylation|>0.3. These DHMRs included 4,686 regions with increased DNA hemi-methylation and 7926 regions with reduced hemi-methylation at either Watson or Crick strands compared to the controls (FIG. 15D). Interestingly, the majority of liver tumor DHMRs did not overlap with DMRs of the same sample set (FIG. 15E), suggesting that DHMRs likely could be used as independent biomarkers for tumor detection. Furthermore, regions with increased hemi-methylation in liver tumor samples were enriched at genomic regions of SINE, CpG islands, promoters and exons, whereas regions with reduced hemi-methylation in liver tumor samples were enriched at intergenic regions, SINEs, LTRs and promoters. Remarkably, the closest genes within 20 kb to these liver tumor HMR (FIG. 15G) and DHMR regions with increased hemi-methylation (FIG. 15H) were enriched in processes linking to cellular metabolism. These results indicate that liver tumor DHMRs and DMRs are independent biomarkers and that liver tissue DHMRs may regulate the expression of genes involved in metabolism.

Develop the Sscf-MeDIP-Seq Method for Analyzing cfDNA Methylomes

Compared to large dsDNA isolated from tumor samples, plasma cfDNAs are a mixture of dsDNA and ssDNA with major fragment sizes about 160-170 nucleotides. Furthermore, some of these DNA are nicked or damaged. All DNA molecules including dsDNA, ssDNA, and damaged DNA were utilized for methylome analysis, which in turn increases the detection sensitivity and reduces the amount of cfDNA needed for analysis (FIG. 16A). Furthermore, this method can also analyze DNA methylation of both Watson and Crick strands, thus allowing analysis of cfDNA hemi-methylation. This method is termed single-stranded (ss) cf-MeDIP-Seq herein. Briefly, after denaturing cfDNA into ssDNA, an adaptor was first ligated to the 3′ end of cfDNA using an ssDNA ligase followed by converting ssDNA into dsDNA by a DNA polymerase. After the ligation of the second adaptor, a small fraction of DNA (10%) was used as input, and the remaining DNA was denatured again and subjected to immunoprecipitation using antibodies against 5-mC. The immunoprecipitated DNA as well as the input DNA were then amplified by PCR for library preparation and sequencing (FIG. 16A).

Using this method, high quality sscf-MeDIP-seq datasets were produced from three groups of samples, controls (individuals without cancer) and individuals with liver or brain cancer. An in-depth analysis of 20 sscf-MeDIP-Seq datasets was performed using cfDNAs from 10 individuals with liver tumor and 10 controls with similar age and sex distributions to gain insight into the performance of sscf-MeDIP-Seq and the properties of cfDNA DMR and DHMRs. First, similar to ssg-MeDIP-seq, sscf-MeDIP-seq signals were depleted at the promoter regions of genes with CpG islands compared to those without CpG islands for all the three groups (FIG. 20B, FIG. 20C, and FIG. 20D). Second, inspection of sscf-MeDIP-seq signals at the TBX2 gene loci also identified a hypermethylated region specific for liver cancer cfDNA samples (FIG. 16B), suggesting these hyper-methylated signals of cfDNAs at this locus are likely from liver tumor cells, but not from normal cells.

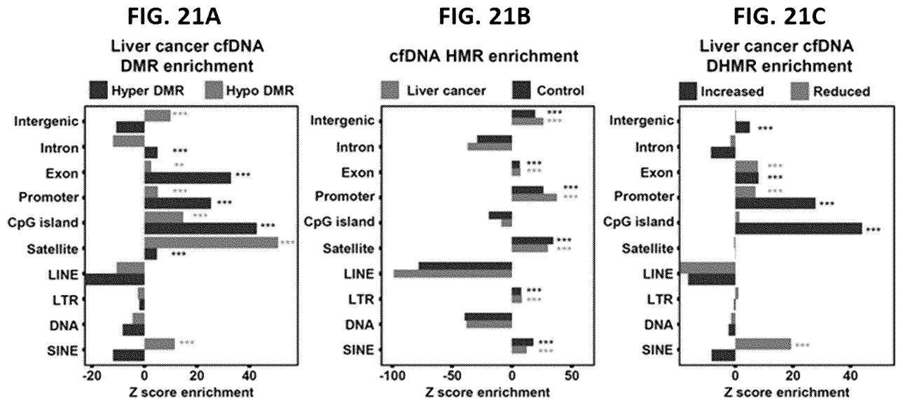

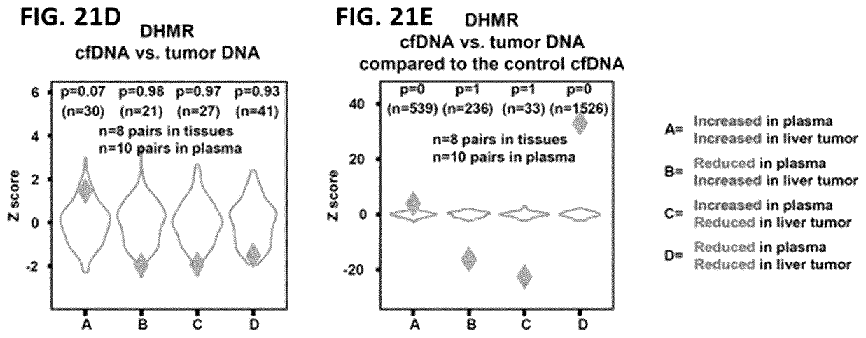

Using the same 2,002,724 blocks, 2,229 hyper-methylated and 5,002 hypo-methylated cfDNA DMRs were identified from 10 liver cancer cfDNA samples compared to the 10 control cfDNA samples (FIG. 15C), with hyper-methylated cfDNA DMRs enriched at CpG islands, promoters, and exons, and hypo-methylated cfDNA DMRs at intergenic regions, satellite DNA, and SINE (FIG. 21A). It was then assessed whether these liver cancer cfDNA DMRs overlapped with liver tumor DNA DMRs identified using ssg-MeDIP-Seq (FIG. 14B, FIG. 14C, FIG. 14D, and FIG. 14E). Both hyper-methylated and hypo-methylated cfDNA DMRs exhibited significant overlap with liver tumor DNA hyper-methylated and hypo-methylated DMRs analyzed by ssg-MeDIP-Seq, respectively (“A” and “D” group in FIG. 15D). In contrast, there were little significant overlaps between hypo-methylated cfDNA DMRs and hyper-methylated liver tumor DNA DMRs or vice versa (“B” and “C” groups FIG. 15D). Together, these results show that cfDNA methylomes generated by sscf-MeDIP-Seq method most likely reflect DNA methylation changes in liver cancer cells.

The Majority of cfDNA DMRs Also do not Overlap with cfDNA DHMRS

cfDNA DHMRs of these 10 liver tumor samples were analyzed compared to the 10 controls (FIG. 16E) and 7,856 and 9,370 regions were identified with increased and reduced hemi-methylation at either Watson or Crick strand, respectively (FIG. 16F). Like tissue samples, cfDNA HMRs from both liver cancer and control samples were enriched at SINEs, satellites, promoters, and exons (FIG. 21B). In contrast, cfDNA DHMRs with increased hemi-methylation specific for liver tumor samples were enriched at CpG islands, promoters, and exons, whereas those with reduced hemi-methylation were enriched at SINEs, promoters, and exons (FIG. 21C). Importantly, like liver tumor DNA DMRs and DHMRs, the vast majority of cfDNA DMRs from liver cancer patients did not overlap with their cfDNA DHMRs (FIG. 16G), indicating that cfDNA DHMRs could also be used as independent biomarkers for tumor detection.

It was assessed whether liver tumor cfDNA DHMRs also showed a significant overlap with liver tumor DNA DHMRs. cfDNA DHMRs with increased hemi-methylation showed significant overlap with tumor DNA DHMRs with increased hemi-methylation (FIG. 21D, “A” group), whereas the cfDNA DHMRs with reduced hemi-methylation group did not show significant overlap with liver tumor DNA DHMRs with reduced hemi-methylation (FIG. 21D, “D” group). One possible reason for the discordance between liver tumor cfDNA DHMRS and DNA DHMRs with reduced hemi-methylation was that it may be challenging to define DHMRs with reduced hemi-methylation due to dramatically different controls used. cfDNA from non-cancer patients was used to define cfDNA DHMRs whereas adjacent non-tumor tissues were used to identify liver tumor DNA DHMRs. To test this idea, liver tumor DNA and cfDNA DHMRs were identified using the same control (control plasma cfDNA samples). In this case, cfDNA DHMRs with both reduced and increased hemi-methylation showed concordance with tumor DNA DHMRs with reduced and increased hemi-methylation, respectively (FIG. 21E). These results indicate that cfDNA DHMRs likely also reflect tumor DNA DHMRs. Taken together, DNA hemi-methylation identified by sscf-MeDIP-Seq likely represent a novel independent biomarker for tumor detection using plasma cfDNA.

### Identification of Cancer Types Using Machine Learning Models Trained Using DMR and DHMRs as Inputs

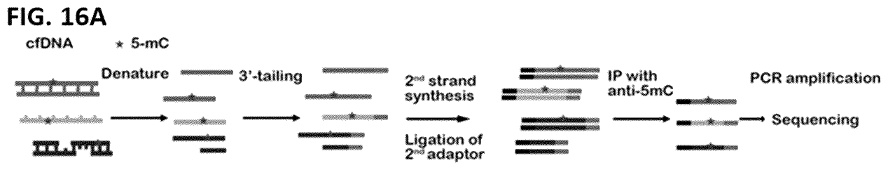

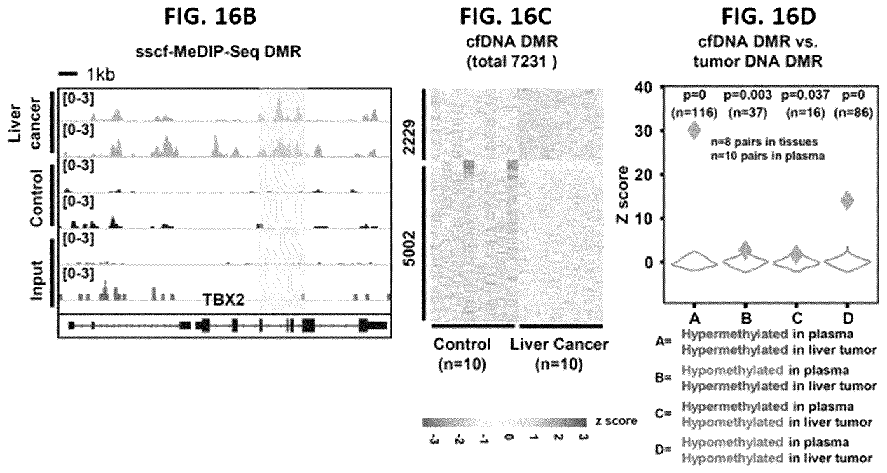

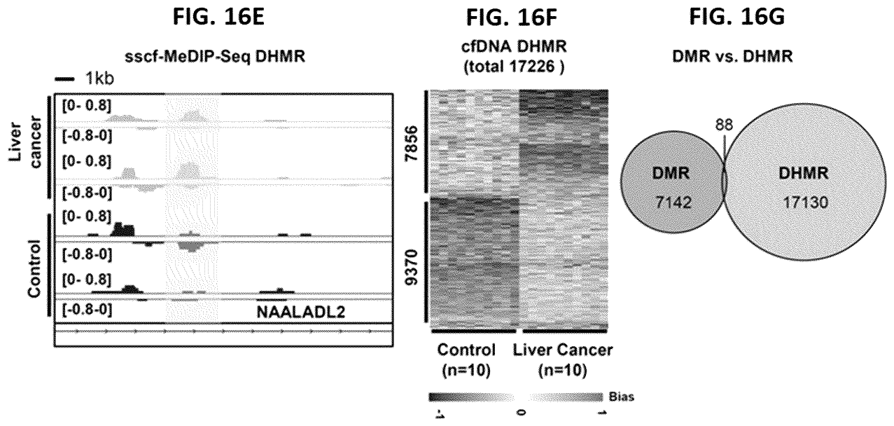

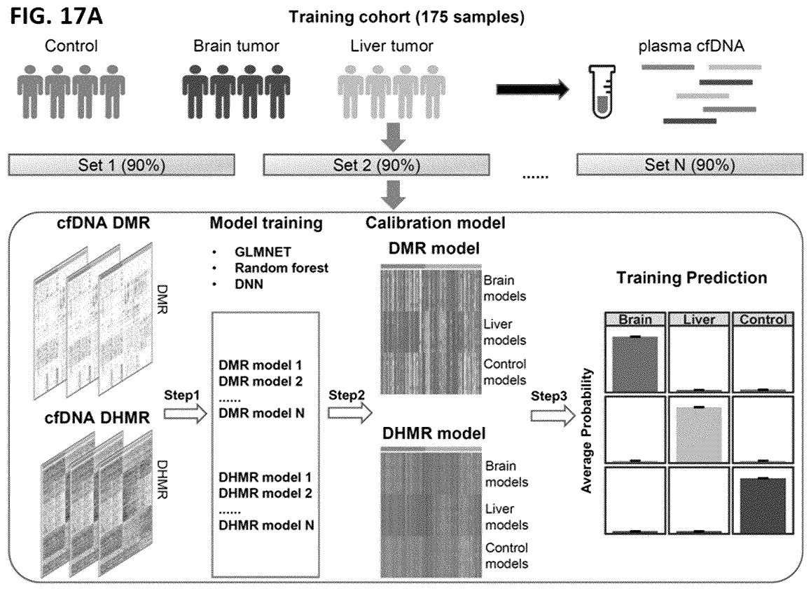

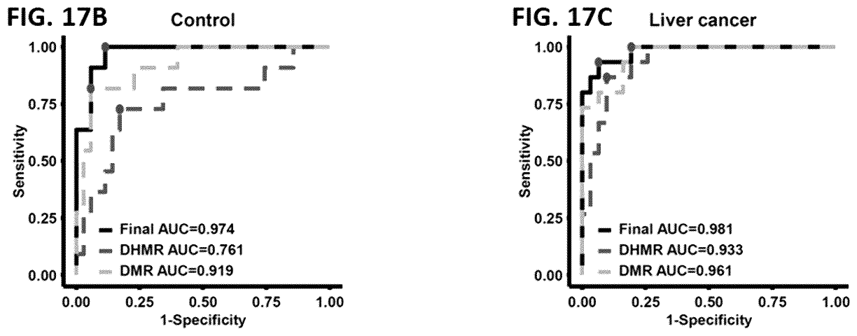

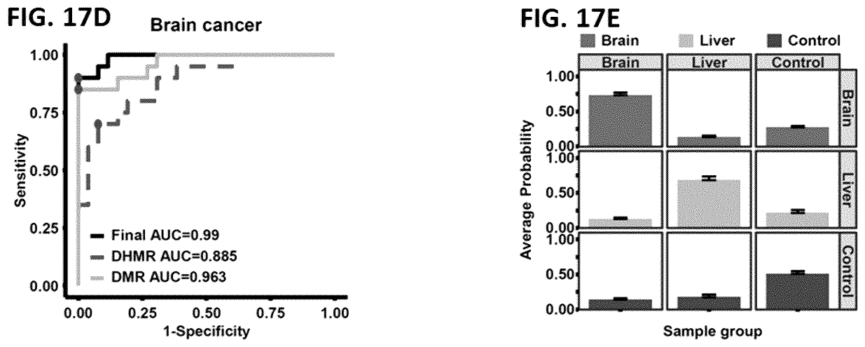

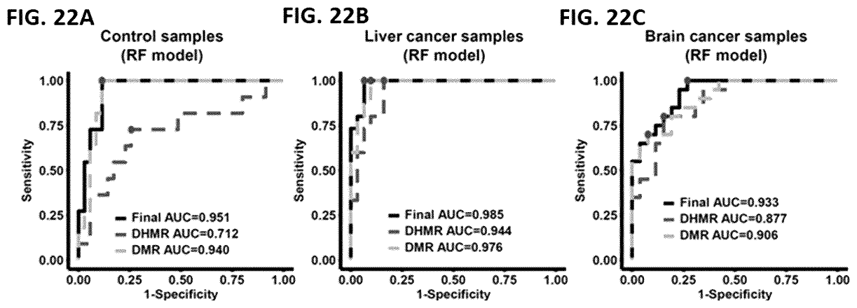

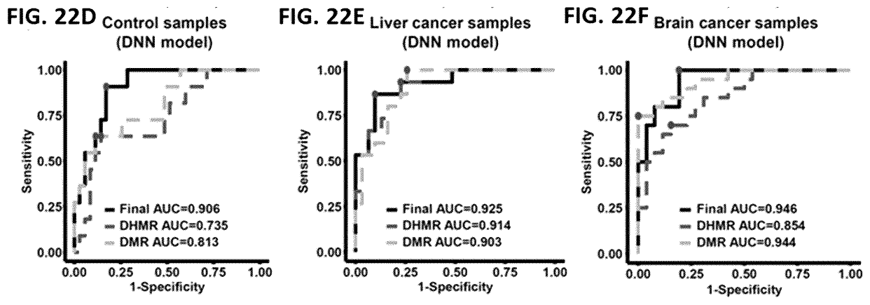

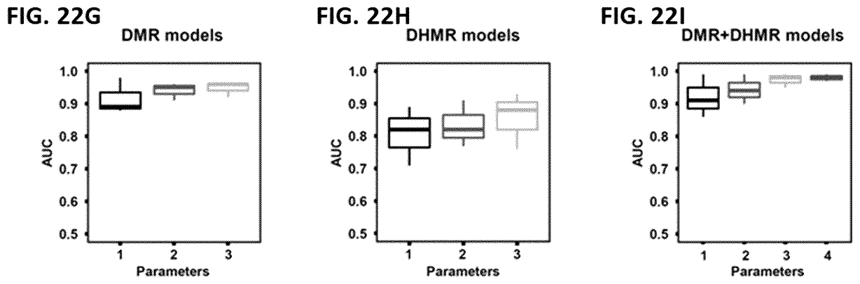

To determine whether sscf-MeDIP-Seq procedures could be used for tumor prediction, cfDNA methylomes of three groups of plasma samples were analyzed: patients with liver (73 samples) or brain cancer (97 samples) and controls (51 samples) (Table 6) and generated a total 221 sscf-MeDIP-Seq datasets including the 20 samples shown in FIG. 16B, FIG. 16C, FIG. 16D, FIG. 16E, FIG. 16F, and FIG. 16G. Of the 221 sscf-MeDIP-Seq datasets generated, 175 datasets including 58 liver cancer and 77 brain cancer samples, and 40 controls were randomly selected and used as the training cohort to train machine learning models of GLMnet, random forest or deep neural network (DNN) (FIG. 17A). All three machine learning models accurately predicted samples in the validation cohorts (46 samples), with GLMnet models showing the best performance (FIG. 17B, FIG. 17C, FIG. 17D, and FIG. 17E, and FIG. 22A, FIG. 22B, FIG. 22C, FIG. 22D, FIG. 22E, and FIG. 22F), highlighting the robustness of the prediction and sscf-MeDIP-Seq datasets. Moreover, as general procedures for model training and sample validation are quite similar for the three models, the description of GLMnet models below was focused on.

TABLE 6
Patient information including cancer type, sex and
age of all 221 cfDNA samples used in this study

| | Training cohort | Validation cohort |
|---|---|---|
| | (N = 175) | (N = 46) |
| Sex and age | | |
| Male | 112 | 28 |
| Female | 62 | 17 |
| Unknown | 1 | 1 |
| Age at diagnosis/recruitment | | |
| Young (<30 years) | 13 | 4 |
| Middle age (30~60 years) | 86 | 21 |
| Old age (>60 years) | 76 | 20 |
| Unknown | 0 | 1 |
| Brain cancer | 77 | 20 |
| IDH WT | 34 | 9 |
| IDH mutant | 43 | 11 |
| Liver cancer | 58 | 15 |
| Controls | 40 | 11 |

To reduce the influence of diversity of individual samples on model training, 90% of the training cohort was randomly sampled 10 times in a balanced way (control, brain and liver cancer), and cfDNA DMRs and DHMRs specific for each sample group were identified in a one-versus-other way, selecting the top DMRs and DHMRs based on the feature importance determined by the GLMnet models. In the beginning, these models were trained using different DMRs and DHMRs of each sample group with DMRs selected by p value and log fold change (LFC) of DNA methylation density and DHMRs selected by feature importance defined by the GLMnet models. There was an increase in model performance when more stringent parameters were used for DMR and DHMR selection (FIG. 22G, FIG. 22H, FIG. 22I). In the end, the top 100 DMRs and 741 DHMRs were selected from the three sample groups for each of the 10 rounds of training using either DMRs or DHMRs as inputs (FIG. 17A). DMR and DHMR models were combined to train a calibration model for the final prediction of each sample in the training cohort. To predict sample identity in the 46-sample validation cohort consisting of 20 brain cancer, 15 liver cancer and 11 control samples, each sample was predicted using 10 models trained with DMRs or DHMRs as the inputs, and the prediction results were combined as the inputs of the calibration model to obtain final prediction probability of each sample.

In general, models based on DMRs alone were better predictors than models based on DHMR alone (FIG. 17B, FIG. 17C, FIG. 17D and FIG. 22A, FIG. 22B, FIG. 22C, FIG. 22D, FIG. 22E, and FIG. 22F). Importantly, when combined, DMR+DHMR-based models yielded more accurate prediction than models based on either DMRs or DHMRs alone (FIG. 17B, FIG. 17C, and FIG. 17D), with AUROC of models using both DMR and DHMR as inputs for brain cancer, liver cancer and controls being 0.99, 0.981, and 0.974, respectively. The average probabilities for identifying brain cancer, liver cancer and control samples using DMR+DHMR-based models were 0.73, 0.69, and 0.51, respectively (FIG. 17E). Finally, two other machine learning models (random forest and DNN) using both DMRs and DHMRs as inputs were also robustly better than models using DMRs or DHMRs alone (FIG. 22A, FIG. 22B, FIG. 22C, FIG. 22D, FIG. 22E, and FIG. 22F). Together, these studies indicate that the sscf-MeDIP-Seq method developed here not only reduces the amount of plasma samples needed to generate high quality genome-wide cfDNA methylomes datasets, but also provides a unique way to analyze both cfDNA DMRs and DHMRs, the latter of which have not been used for tumor detection, to increase the accuracy of tumor detection.

### Evaluate the Sensitivity of the Sscf-MeDIP-Seq Method

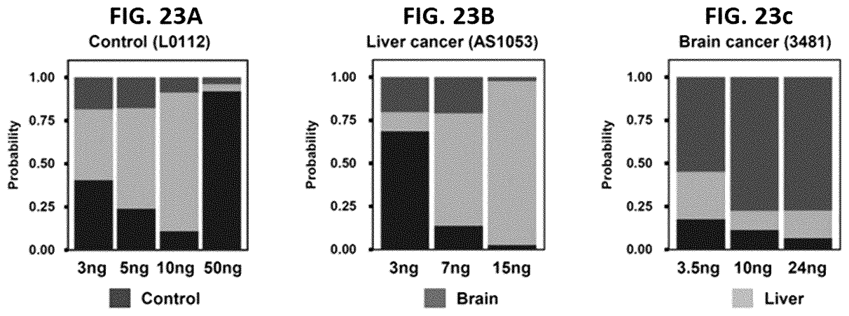

The amount of cfDNA in plasma differs from sample to sample, with early-stage tumors in general releasing less circulating tumor DNA into blood than late-stage tumors. Therefore, the amount of cfDNA needed for the generation of high quality sscf-MeDIP-Seq datasets for tumor prediction was tested. Briefly, two cfDNA samples were randomly chosen, with each from individuals with liver and brain tumors. sscf-MeDIP-Seq datasets were generated using different amounts of DNA isolated from each sample. For instance, three sscf-MeDIP-Seq datasets were generated from three different amounts of cfDNA from one brain (3.5 ng, 10 ng, 24 ng) and one liver cancer sample (3 ng, 7 ng, 15 ng) (FIG. 23A, FIG. 23B, FIG. 23C). The GLMnet models trained in FIG. 16A, FIG. 16B, FIG. 16C, FIG. 16D, FIG. 16E, FIG. 16F, and FIG. 16G were applied to predict sscf-MeDIP-Seq samples from the different amounts of input DNAs. The models could reliably predict brain tumor samples using three different amounts of cfDNAs (FIG. 23A), liver cancers from the two higher amounts of cfDNA (FIG. 23B). For the healthy control samples, the cfDNA amount is usually low ranging from 3-10 ng from 1 ml plasma and a reliable library can generally be obtained from about 3 ng of cfDNA. These results indicate that the amount of cfDNA isolated from different subjects varies, with higher input cfDNA generally yielding better quality of sscf-MeDIP-Seq datasets for prediction. All sscf-MeDIP-Seq datasets of 221 cfDNA samples were generated using cfDNAs purified 300 μl to 500 μl of plasma samples.

Differentiate Glioma Subtypes by cfDNA Methylomes

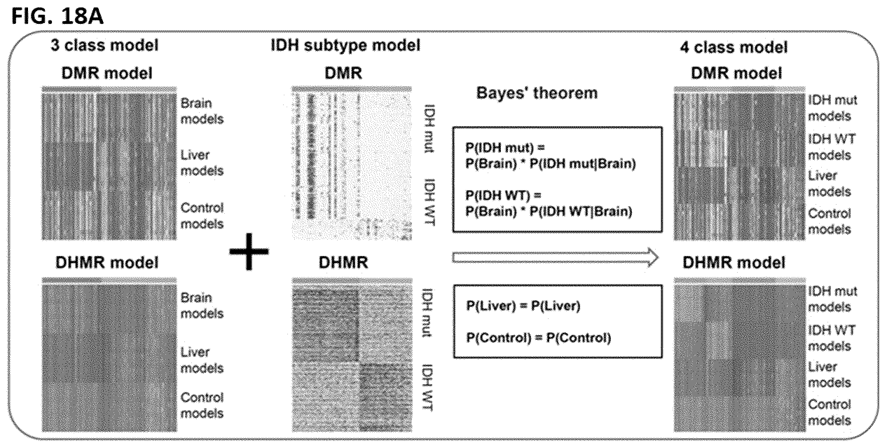

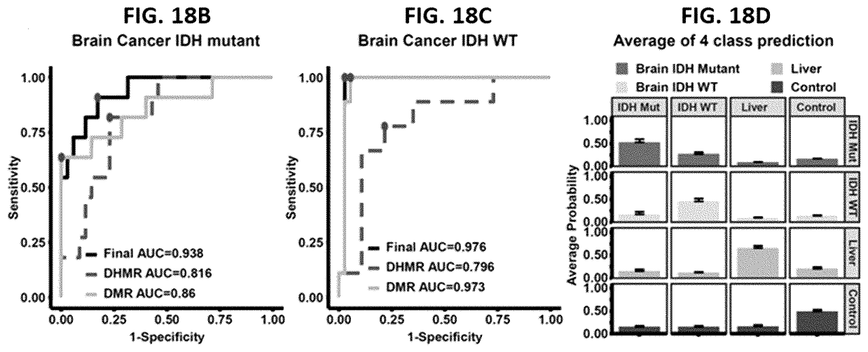

It was also assessed whether cfDNA methylome analysis can be used to differentiate the subtypes of brain tumors. Of 77 cfDNA samples from brain tumor patients in the training cohort, 43 samples were from patients with IDH mutations and 34 with IDH wild type tumors. To train brain tumor subtype models, the 77 brain tumors samples of the training cohort were separated into IDH wild type (34 samples) and IDH mutant groups (43 samples), and same procedures outlined above were followed to train the GLMnet models using either DMRs or DHMRs as inputs. These brain subtype models were then combined with the three-class model (brain cancer, liver cancer and control) based on Bayes's theorem to expand the model for four samples groups (IDH WT and IDH mutant brain cancer, liver cancer, and control) (FIG. 18A). Using the four-sample class model, the prediction probability of each sample in the validation cohort was calculated. As shown in FIG. 18B and FIG. 18C., IDH mutant and IDH wild type brain tumor subtypes could be identified accurately, with the models using both DMR and DHMR having the best performance (AUROC of 0.938 and 0.976 for IDH mutant and IDH WT, respectively). Finally, the average probabilities of IDH mutation gliomas, IDH wild type gliomas, liver cancer, and control groups were 0.52, 0.46, 0.65, and 0.49, respectively (FIG. 18D). Together, these studies indicate that models using both DMRs and DHMRs as inputs could also be used identify glioma subtypes accurately.

cfDNA DMRs and DHMRs are Associated with Genes Whose Gene Expressions in Tumor Tissues Predict Patient Survival

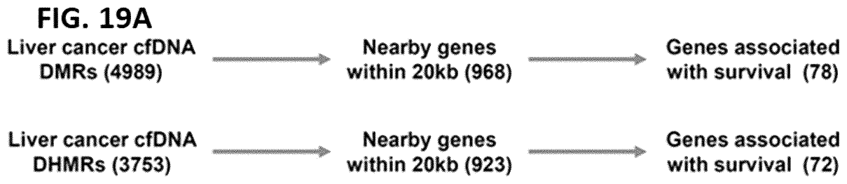

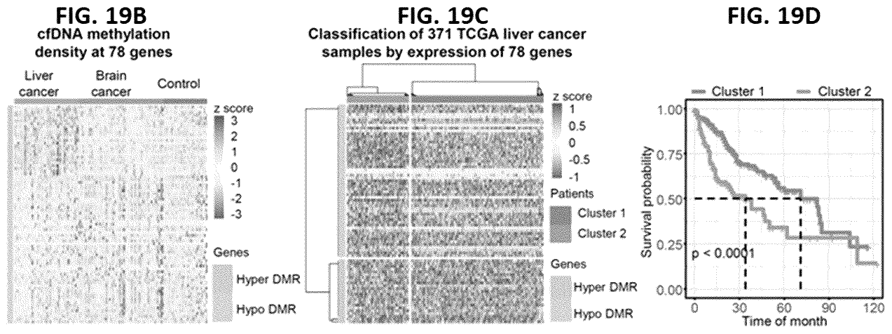

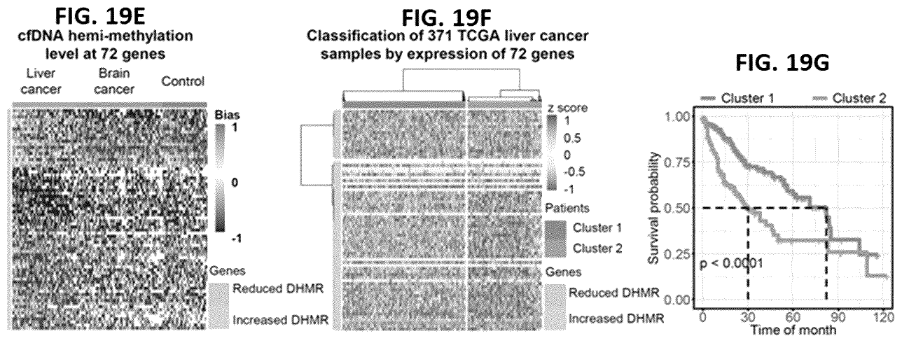

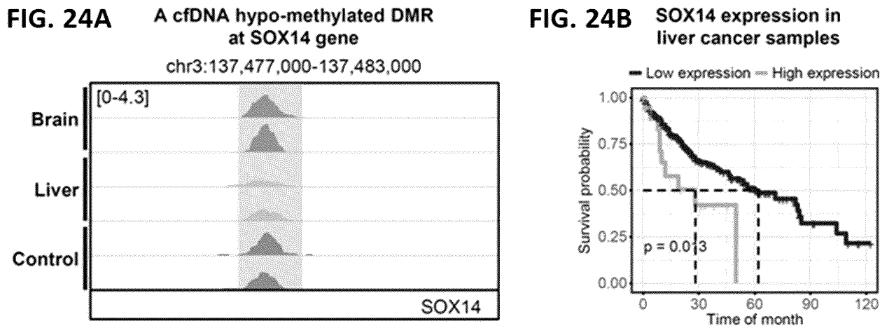

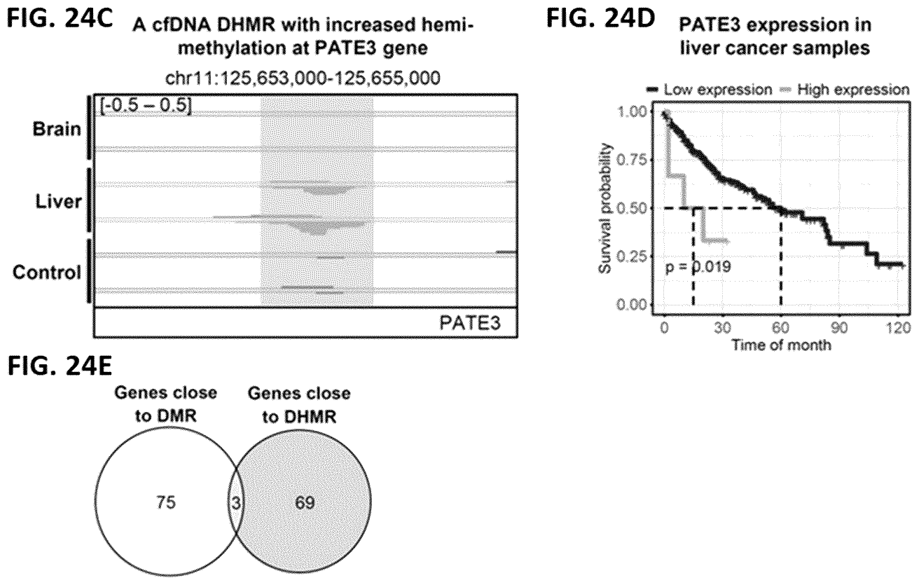

Promoter and enhancer DNA methylation is associated with gene transcription. To probe the potential relationship between cfDNA DMRs and DHMRs and gene expression in tumor samples, each of the liver cancer specific 4989 cfDNA DMRs were annotated, which were identified by comparing cfDNA methylomes of all 58 liver cancer samples to those from control and brain tumor samples in the training cohorts, to their closest genes and identified 968 genes whose promoters were within 20 Kb of one of these DMRs. It was then assessed whether the expression of each of the 968 genes in 371 liver tumor samples in the TCGA database was associated with patient survival (FIG. 19A). For instance, a hypo-methylated DMR at the SOX14 gene locus specific for liver cancer compared to controls and brain tumor samples was identified (FIG. 24A). Furthermore, patients with high expression of SOX14 in the 371 TCGA liver cancer dataset was associated with poor survival compared to those with lower expression (FIG. 24B). Through the analysis, of the 968 genes with at least one liver cancer specific cfDNA DMR nearby, the expression of 78 genes in liver cancer tissues was associated with patient survival. Of these 78 genes, 26 genes were enriched with hyper-methylated cfDNA DMRs, whereas 52 genes were close to hypo-methylated cfDNA DMRs (FIG. 19B). Next, it was assessed whether the expression of these 78 genes could be used to cluster the 371 TCGA liver cancer patient samples using unsupervised clustering analysis. 371 liver cancer samples could be separated into two clusters. Interestingly, genes close to the hypo-methylated cfDNA DMRs are highly expressed in “Cluster 2” liver tumor samples compared to “Cluster 1” (FIG. 19C). In contrast, genes close to hyper-methylated cfDNA DMRs are highly expressed in “Cluster 1” patient samples. Importantly, patients in these two clusters showed dramatically different survival times, with the median survival of patients in Cluster 1 and Cluster 2 being ˜70 and 30 months, respectively (FIG. 19D). The same procedures were applied and 72 genes were identified close to liver cancer specific cfDNA DHMRs and whose expression in liver tumor tissues is associated with patient survival (FIG. 24C and FIG. 24D). Furthermore, only 3 of these 72 genes overlapped with the 78 genes close to liver cancer cfDNA DMRs, consistent with the idea that liver cancer DMRs and DHMRs do not overlap. Importantly, the expression of these 72 genes in 371 liver tumor samples also separated these samples into two groups with a significant difference in survival (FIG. 19E, FIG. 19F, FIG. 19G). Together, these results indicate that a significant fraction of liver cancer specific cfDNA DMRs and DHMRs identified in this study are likely associated with changes in the expression of nearby genes in tumor cells, which in turn may contribute to tumorigenesis.

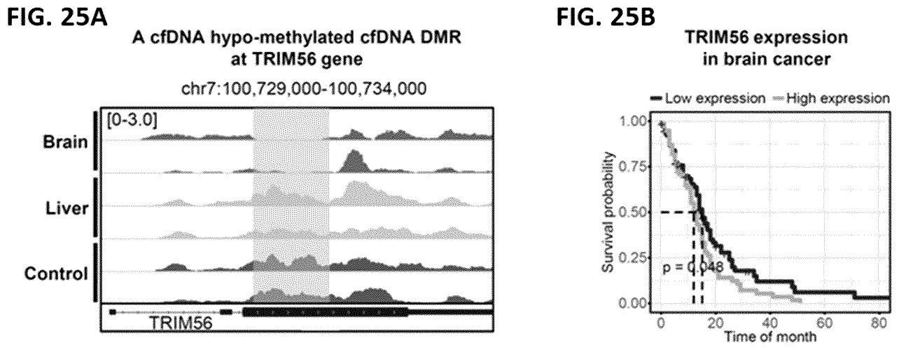

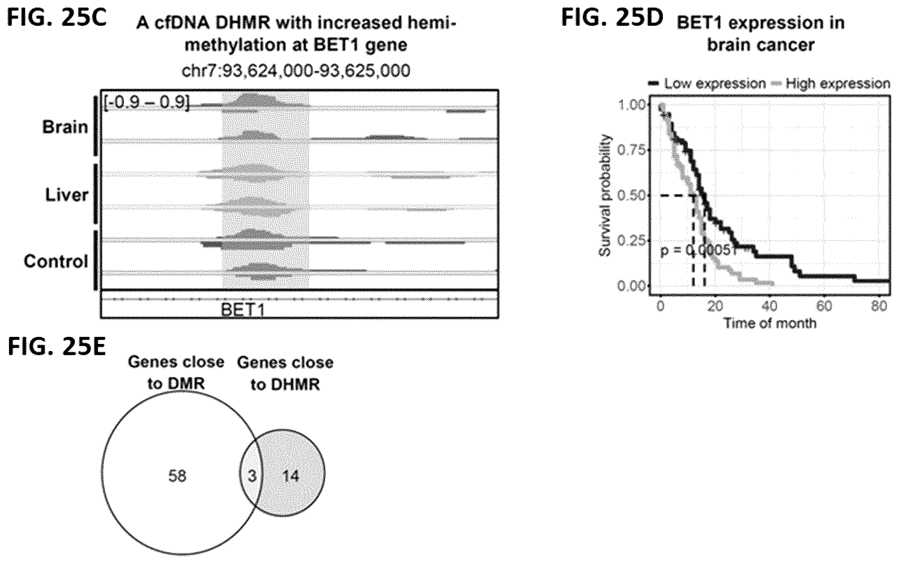

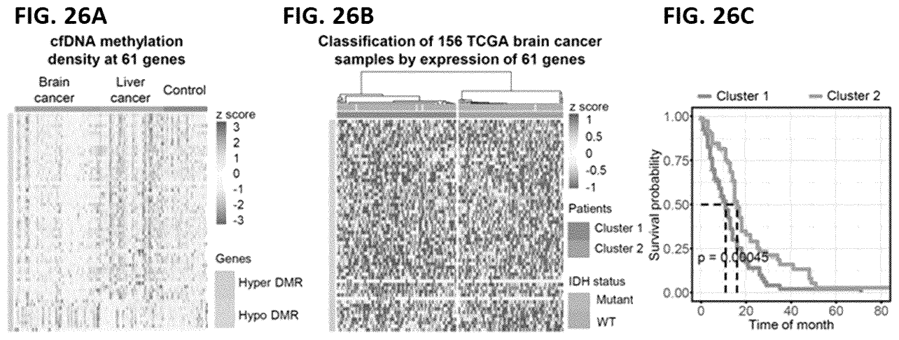

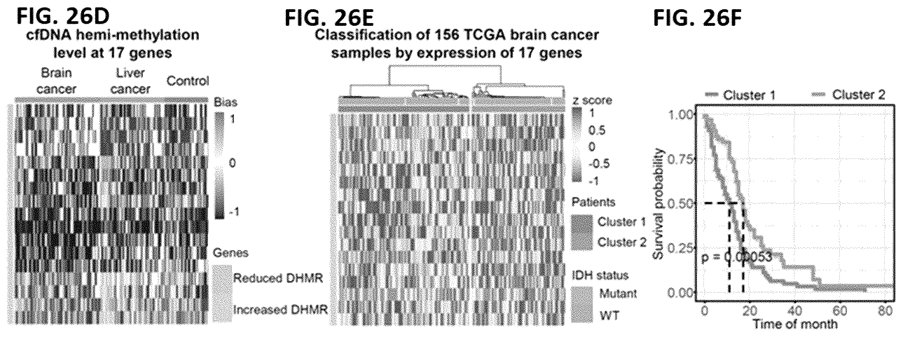

The same approach identified 61 and 17 genes with at least one brain tumor specific cfDNA DMR or DHMR nearby, respectively, with only 3 genes shared between these two groups of genes (FIG. 25A, FIG. 25C, and FIG. 25E). Furthermore, the expression of these genes in primary brain tumor tissue samples was associated with patient survival (FIG. 25B, FIG. 25C, and FIG. 25D). Importantly, the expression of the 61 genes close to a brain cancer specific cfDNA DMR could also separate 156 brain tumor samples from the TCGA database into two different clusters with patients in “Cluster 2” showing better survival than those in “Cluster 1” (FIG. 26A, FIG. 26B, and FIG. 26C). Interestingly, patient samples with IDH mutations were enriched in “Cluster 2” (Fisher test, OR=16.6, p=0.001), and it is known that brain tumor patients with IDH mutations have a favorable outcome compared to glioma patients with wild type IDH gliomas. Similarly, the expression of the 17 genes with at least one brain cancer cfDNA DHMR nearby also separated brain tumor samples into two different groups with different survival (FIG. 26D, FIG. 26E, and FIG. 26F). Together, these studies indicate that cfDNA DMRs and DHMRs for both liver and brain tumor patients are likely associated with changes in expression of genes involved in tumorigenesis.

DNA cytosine methylation plays an important role in gene regulation, chromatin maintenance, and genomic stability. Aberrant DNA methylation occurs in a variety of cancers. Therefore, DNA methylomes in cancer tissues have been used for tumor classification and detection. In this study, the ssg-MeDIP-seq procedures for analyzing methylomes of genomic DNA as well as sscf-MeDIP-Seq for analyzing plasma cell free DNA (cfDNA) were developed. These new and markedly simplified MeDIP-seq procedures greatly reduce the amount of DNA and time needed for the generation of MeDIP-seq datasets. Importantly, these methods allow the analysis of symmetric DNA methylation as well as DNA hemi-methylation, a novel epigenetic mark that has not been used for tumor detection, at the same time.

To optimize and simplify MeDIP-seq procedures for analyzing DNA methylomes of genomic DNA isolated from normal or tumor tissues, pA-Tn5 loaded with one adaptor was utilized, which tagments dsDNA into small fragments. In this way, the sonication step for shearing DNA into small fragments was not needed, which is the first step in previously published MeDIP-seq procedures. Furthermore, because of tagmentation, MeDIP-seq libraries are generated through a simple PCR step without other complicated and inefficient steps such as primary ligation and/or T/A tailing described in published MeDIP-seq procedures. Because pA-Tn5 attaches the adaptor only to the 5′ end of each strand covalently, another adaptor is used to replace the first adaptor at the 3′ end following tagmentation. These modifications allow the generation of high-quality MeDIP-seq libraries from 100 ng tumor DNA in less than two days. Importantly, this method allows the measurement of DNA methylation at both Watson and Crick strands separately. Therefore, ssg-MeDIP-Seq can measure both DNA methylation density and hemi-methylated regions at the same time.

Following DNA replication, symmetrically methylated CpG will become hemi-methylated as the newly synthesized DNA strand is unmethylated, but will become fully methylated during S phase to maintain DNA methylation patterns. Recently, it has been shown that about 10% of 3 million CpGs sites are hemi-methylated and these hemi-methylated CpGs remain hemi-methylated following DNA replication, indicating that DNA hemi-methylation is a novel epigenetic mark. 260,055 and 325,866 HMR regions were identified in 8 liver tumor samples and 8 adjacent controls tissues, which are about 10-15% of the total potential methylated blocks used for analysis. 12,592 differentially hemi-methylated regions (DHMRs) were also identified by comparing HMRs in liver tumor DNA to DNA of adjacent non-tumor tissues. Interestingly, these DHMRs are enriched at genes involved in metabolism. Therefore, it is possible that these liver specific DHMRs regulate the expression of genes involved in metabolism in liver cancer. Previously, it has been shown that HMRs are also important for gene regulation. The majority of liver tumor DNA DMRs and DHMRs do not overlap. These results suggest that liver cancer DHMRs identified in this study can serve as biomarkers for liver tumor DNA samples independent of DMRs.

In contrast to large dsDNA fragments for genomic DNA, cfDNAs are a mixture of dsDNA, ssDNA, and damaged DNA, which are similar to ancient DNA samples. The disclosed sscf-MeDIP-Seq procedures were developed with modifications that allow the inclusion of double stranded, single-stranded, and damaged cfDNA for methylation analysis, and as such the generation of MeDIP-seq libraries from cfDNA purified from 300-500 μl plasma samples, with as little as 30 μl plasma for some samples. Therefore, this method requires much less plasma for analyzing cfDNA methylomes compared to the published cfDNA methylome analysis. Importantly, the sscf-MeDIP-Seq method can also measure cfDNA methylation on Watson and Crick strands separately, which makes it possible to analyze cfDNA hemi-methylation. Like liver tumor DNA DMRs and DHMRs, most liver cancer cfDNA DMRs do not overlap with cfDNA DHMRs. These results indicate that liver cancer cfDNA DMRs and cfDNA DHMRs are also independent biomarkers. Indeed, by analyzing cfDNA methylomes of 221 plasma samples from three groups of individuals, machine-learning models trained with both cfDNA DMRs and DHMRs as inputs yield better performance for the prediction of these three groups of samples in the validation cohort than models trained with cfDNA DMRs or DHMRs alone. These results indicate that DHMRs are indeed independent biomarkers for the detection of samples from individuals without cancer, or with brain or liver cancer. Recently, it has been shown that genome-wide cfDNA methylome analysis, which relies on DMRs, gives rise to improved cancer detection than other methods including mutations and cfDNA pan features.

### Materials and Methods

### Biospecimens

Hepatocellular cancer patients' samples were from an IRB-approved, hospital-based prospective study conducted at Columbia University Irving Medical Center (CUIMC) that recruited liver cancer patients (>18 years older) from October 2008 to July 2014. Brain cancer patients' samples were collected as part of an IRB-approved protocol to collect, bank and distribute de-identified samples from brain tumor patients at CUIMC. Subjects without cancer were recruited from advertisements around CUIMC also with IRB approval. All subjects provided blood samples which were rapidly centrifuged to obtain plasma which was aliquoted and frozen at −80° C. until use. Basic epidemiologic variables were obtained by a structured questionnaire while clinical information on patients was obtained from medical records. Written informed consent was obtained from all participants and this research project was approved by the Columbia University Institutional Review Board.

### Protein, Antibody and Reagents

Purification of pA-Tn5 and pA-Tn5-oligo complex assembly used for analysis of methylation of tumor DNA were as described previously (Li, Z., et al., Nat Protoc 16, 5739. (2021) and Li, Z. M. et al., Sci Adv 6, doi: ARTN cabb5820. (2020)). Antibodies against 5-mC (33D3) were purchased from Diagenode (C15200081). Mouse IgG used to bridge antibodies against 5-mC and pA-TN5 was purchased from Active Motif (53017), and tRNA was purchased from Sigma (R1753)

### Preparation of Genomic DNA

Genomic DNA was extracted from frozen tumor and adjacent tissues by standard proteinase K and RNase treatment followed by phenol and chloroform extraction. Tagmentation of genomic DNA was performed as previously described with minor modifications (Li, Z., et al., Nat Protoc 16, 5739, (2021) and Li, Z. M. et al., Sci Adv 6, doi: ARTN cabb5820, (2020)). In brief, 100 ng of DNA and 1.5 μl of pA-Tn5-AA complex were mixed in the Tagmentation buffer (5 mM TAPS-NaOH pH8.5, 5 mM MaCl2, 10% DMF), and were incubated in 37° C. with gentle shaking for 30 min. DNA was then purified by CHIP DNA clean kit (Zymo 11-379C), and oligo replacement and GAP repair followed the same procedures as described (Li, Z., et al., Nat Protoc 16, 5739, (2021) and Li. Z. M. et al., Sci Adv 6, doi: ARTN cabb5820, (2020)). The processed DNA was then subjected to immunoprecipitation using antibodies against 5-mC described below.

### MeDIP-Seq Data Analysis

MeDIP-Seq libraries were sequenced using a paired-end method on Illumina Nextseq 500/550 or NOVA-seq platforms Adaptor sequences of all raw reads were removed by Cutadapt and reads <10 nt were removed. Reads passed through these cleanup steps were then mapped to the human reference genome (hg19) by Bowtie2. Duplicate reads were removed using Sambamba software. Read coverage in a bin of 1 bp was calculated from filtered bam files by deepTools2 and then normalized with total number of filtered reads into reads per million (RPM).

Protein coding gene annotation was downloaded from GENCODE (v28) and the CpG islands annotation was downloaded from UCSC Table Browser. Protein coding genes were then classified into genes with and without CpG islands based on the overlap with their promoters ([−3 kb, 3 kb] surrounding TSS). Normalized reads density (RPM) of MeDIP-Seq was used to calculate from transcription start sites (TSS) to transcription termination sites (TTS) for each class of genes respectively by deepTools2.

Differentially Methylated Region (DMR) Identification for Genomic DNA and Plasma cfDNA

Recently, it has been shown that 2,002,724 blocks each with at least four CpG dinucleotides can monitor DNA methylation from 205 tissues across multiple conditions. Therefore, 2,002,724 blocks with at least four CpG dinucleotides were used to identify DMRs and DHMRs. To identify DMR of genomic DNA of liver tumor tissues and adjacent non-tumor tissues, ssg-MeDIP-seq datasets were compared from eight liver tumor samples to each corresponding adjacent non-tumor tissues, and 11,930 hyper-methylated DMRs and 12,974 hypo-methylated DMRs were identified by QSEA with a cutoff of p<0.01 and log 2 (fold change)>1. For comparison, TCGA LIHC methylation datasets of Illumina 450K CpG array were downloaded. Methylation level β values of 50 liver tumor samples were compared with their corresponding adjacent samples, and 10,362 hypermethylated DMRs and 46,969 hypomethylated DMRs were identified using the T-test with a cutoff of Bonferroni adjusted p<0.05. To estimate the significance of overlaps between liver cancer DNA DMRs identified in this study and DMRs identified using TCGA datasets, 100 permutations were performed by bedtools with the command “bedtools shuffle—incl regulation.bed”. 5207 concordantly hyper-methylated and 1472 concordantly hypo-methylated DMRs between the liver cancer DMRs found in this study and TCGA liver tumor samples using 450K methylation arrays were identified. The observed number of overlapping DMR (5207 and 1472) was compared with the null hypothesis distribution generated from corresponding 100 permutations and standard normalized to Z score (P value is calculated by the null distribution in a one-sided way). Both of the concordant hyper-methylated and hypo-methylated DMRs showed significantly (p=0 for 5207 hyper-methylated DMRs and p=0 for 1472 hypo-methylated DMRs, respectively) higher enrichment than random permutation distributions (FIG. 14D).

To identify cfDNA DMRs, cfDNAs sscf-MeDIP-Seq datasets were compared from 10 liver cancer patients with those from 10 control individuals without cancer using QSEA with a cutoff of p<0.01 and log 2 (fold change)>1. To evaluate whether these cfDNA DMRs were also found in liver tumor DNA DMRs, the overlap between cfDNA DMRs were compared with tumor DNA DMRs identified using ssg-MeDIP-seq in this study. Similarly, discordantly methylated regions (hyper-methylated in one setting vs hypo-methylated in another setting) between cfDNA DMR and DMRs on tumor DNAs from this study were also compared. Subsequently, the observed numbers of concordantly and discordantly overlapped DMRs were compared with the null distributions generated from the corresponding 100 random permutations. The observed numbers of overlapping DMRs were normalized to Z score by null distributions and p values were calculated in a one-sided way to evaluate the significance of overlap.

Differentially Hemi-Methylated Region (DHMR) Identification for Genomic DNA and Plasma cfDNA

The same 2,002,724 blocks from Loyfer et al., (Nature 613, 355-364, (2023)). were also used to identify hemi-methylated regions (HMRs) and differentially hemi-methylated regions (DHMR). Briefly, hemi-methylation level at each block was calculated as “bias”

$$= \frac{Watson - Crick}{Watson + Crick} ,$$

which ranges from −1 to 1. Watson and Crick represent ssg-MeDIP-Seq or sscf-MeDIP-Seq sequence reads of Watson and Crick strand, respectively. HMRs were identified using a cutoff of the absolute bias greater than 0.3. To identify liver tumor DNA DHMR, cight ssg-MeDIP-Seq datasets from liver tumor were compared to their adjacent non-tumor tissues (FIG. 15D). To identify cfDNA DHMRs for detailed analysis, cfDNA from 10 individuals with liver tumor and 10 control individuals without liver cancer were compared (FIG. 16F). T-test was used on each DNA methylation block with the cutoffs of p<0.01 and delta bias >0.3 and minimum bias at each block >0.3. Using this cutoff, 12, 612 liver tumor DNA DHMRs (FIG. 15D) and 17, 226 liver tumor cfDNA DHMRs were identified (FIG. 16F).

### Machine Learning Models

To detect and classify tumors by cfDNA methylomes, a regularized regression model of Lasso and Elastic-Net Regularized Generalized Linear Model (GLMnet) was used as the final model, and two other machine learning models were also tested, Deep Neural Network (DNN) and Random Forest (RF). To evaluate the performance of machine learning models, the receiver operating characteristic (ROC) curve was plotted in sensitivity against (1-specificity) for each class, where sensitivity=(true positives/total positives) and specificity=(true negatives/total negatives). The area under ROC curve (AUROC) were calculated for comparison by “PROC” package in R.

For GLMnet model, the elastic net penalty is controlled by a, bridging the gap between lasso regression (α=1) and ridge regression (α=0). In this study, α and λ were tuned over a grid of values to optimize the model from 0 to 1 in increments of 0.1, and the family function was set to “glmnet” for regression. For random forest model, the number of subset features for random selection was tuned over a grid of values by from 2 to the squared root of total number of features (DMRs or DHMRs), and 1000 trees were generated in each round. Model training was performed using 10-fold cross-validation and applied by “caret” package in R. For the Deep Neural Network, the models consisted of an input layer, an output layer of three nodes for the predicted probability of each of the three types, and two hidden layers with 64 and 32 nodes. To process the input signals, the activation function for hidden layers were linear functions; and the output layer was the “softmax” function for multinomial classification. The L2 penalty was set to 0.1 for regularization of the hidden layers to reduce the risk of overfitting. The DNN models by “Keras” package were applied in R.

cfDNA DMRs or DHMR are Associated with Patient Survival

Cancer specific DMRs and DHMRs were annotated to their closest genes within 20 kb. The RNA expression in RSEM value (RNA-Seq by Expectation Maximization) and patient's clinical data were downloaded from TCGA-LIHC project (371 liver cancer patient samples) and TCGA-GBM project (156 brain cancer patients). Based on the median RNA expression of each of these nearby genes in these cohorts of patient samples, liver or brain cancer samples were separated into two groups, high and low expression. The cox proportional hazards model was performed for each of the nearby genes to evaluate hazard ratio on patients' survival. The genes whose expression in these cancer samples is associated with patient survival were chosen for further analysis.

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

### EMBODIMENTS

Embodiment 1. A method of preparing a DNA library from isolated DNA, the method comprising:
incubating the isolated DNA with a fusion protein comprising protein A and a Tn5 transposase (pA-Tn5) to thereby generate DNA fragments, and isolating the DNA fragments to form the DNA library.

Embodiment 2. The method of embodiment 1, wherein the DNA library contains DNA fragments that are about 100 nucleotides to about 300 nucleotides in length.

Embodiment 3. The method of embodiment 1 or 2, wherein about 100 ng of isolated DNA is incubated with the pA-Tn5.

Embodiment 4. The method of any one of the previous embodiments, wherein the pA-Tn5 attaches a first tag to the 5′ end of the DNA fragments.

Embodiment 5. The method of embodiment 4, further comprising ligating a second tag to the 3′ end of the DNA fragments through oligo-replacement.

Embodiment 6. The method of any one of the previous embodiments, wherein the isolated DNA is from a cell.

Embodiment 7. The method of any one of the previous embodiments, wherein the method does not use an antibody or other DNA-targeting molecule to target the pA-Tn5 to the DNA.

Embodiment 8. The method of any one of the previous embodiments, further comprising amplifying one or more of the DNA fragments from the DNA library with polymerase chain reaction (PCR).

Embodiment 9. The method of embodiment 8, further comprising sequencing the amplified DNA fragments.

Embodiment 10. A method of identifying a modification of interest in isolated DNA, the method comprising:
incubating the isolated DNA with pA-Tn5 to thereby generate DNA fragments;
isolating DNA fragments having the modification of interest; and
identifying the modification of interest from the DNA fragments.

Embodiment 11. The method of embodiment 10, wherein the isolating comprises immunoprecipitating the DNA fragments having the modification of interest with an antibody.

Embodiment 12. The method of embodiment 11, wherein the antibody is a methylation-specific antibody.

Embodiment 13. The method of embodiment 12, wherein the methylation-specific antibody is an anti-5mC antibody.

Embodiment 14. The method of any one of embodiments 10-13, wherein the identifying comprises amplifying immunoprecipitated DNA fragments with polymerase chain reaction (PCR).

Embodiment 15. The method of any one of embodiments 10-14, wherein the identifying comprises sequencing immunoprecipitated DNA fragments.

Embodiment 16. The method of any one of embodiments 10-15, wherein the pA-Tn5 attaches a first tag to the 5′ end of the DNA fragments.

Embodiment 17. The method of embodiment 16, further comprising ligating a second tag to the 3′ end of the DNA fragments through oligo-replacement.

Embodiment 18. The method of any one of embodiments 11-17, further comprising denaturing the DNA fragments into single stranded DNA fragments prior to the immunoprecipitating.

Embodiment 19. The method of any one of embodiments 10-18, wherein the DNA fragments are about 100 nucleotides to about 300 nucleotides in length.

Embodiment 20. The method of any one of embodiments 10-19, wherein about 100 ng of isolated DNA is incubated with the pA-Tn5.

Embodiment 21. The method of any one of embodiments 10-20, wherein the isolated DNA is from a cell.

Embodiment 22. The method of any one of embodiments 10-21, wherein the method does not use an antibody or other DNA-targeting molecule to target the pA-Tn5 to the DNA.

Embodiment 23. A kit comprising:
pA-Tn5, and
instructions for use in performing the methods of any one of embodiments 1-22.

Embodiment 24. The kit of embodiment 23, further comprising one or more oligonucleotides.

Embodiment 25. The kit of embodiment 24, wherein the one or more oligonucleotides comprise oligonucleotides adapted to serve as a 3′ tag.

Embodiment 26. The kits of embodiment 24 or 25, wherein the one or more oligonucleotides comprise oligonucleotide primers.

Embodiment 27. The kit of any one of embodiments 23-26, further comprising an antibody that binds to a DNA modification.

Embodiment 28. The kit of embodiment 27, wherein the antibody is a methylation-specific antibody.

Embodiment 29. The kit of embodiment 28, wherein the methylation-specific antibody is an anti-5mC antibody.

Embodiment 30. A method of diagnosing cancer in an individual, the method comprising:
incubating DNA obtained from the individual with pA-Tn5 to thereby generate DNA fragments;
isolating DNA fragments having a modification of interest; and
identifying the position of the modification of interest from the DNA fragments, thereby diagnosing cancer.

Embodiment 31. The method of embodiment 30, wherein the isolating comprises immunoprecipitating the DNA fragments having the modification of interest with an antibody.

Embodiment 32. The method of embodiment 31, wherein the antibody is a methylation-specific antibody.

Embodiment 33. The method of embodiment 32, wherein the methylation-specific antibody is a 5mC antibody.

Embodiment 34. The method of any one of embodiments 30-33, wherein the identifying comprises amplifying immunoprecipitated DNA fragments with polymerase chain reaction (PCR).

Embodiment 35. The method of any one of embodiments 30-34, wherein the identifying comprises sequencing immunoprecipitated DNA fragments.

Embodiment 36. The method of any one of embodiments 30-35, wherein the pA-Tn5 attaches a first tag to the 5′ end of the DNA fragments.

Embodiment 37. The method of embodiment 36, further comprising ligating a second tag to the 3′ end of the DNA fragments through oligo-replacement.

Embodiment 38. The method of any one of embodiments 31-37, further comprising denaturing the DNA fragments into single stranded DNA fragments prior to immunoprecipitating.

Embodiment 39. The method of any one of embodiments 30-38, wherein the DNA fragments are about 100 nucleotides to about 300 nucleotides in length.

Embodiment 40. The method of any one of embodiments 30-39, wherein about 100 ng of DNA is incubated with the pA-Tn5.

Embodiment 41. The method of any one of embodiments 30-40, wherein the DNA is obtained from a cell, plasma, or blood.

Embodiment 42. The method of any one of embodiments 30-41, wherein the method does not use an antibody or other DNA-targeting molecule to target the pA-Tn5 to the DNA.

What is claimed:

1. A method of preparing a DNA library from isolated DNA, the method comprising:
incubating the isolated DNA with a fusion protein comprising protein A and a Tn5 transposase (pA-Tn5) to thereby generate DNA fragments, and
isolating the DNA fragments to form the DNA library.

2. The method of claim 1, wherein the DNA library contains DNA fragments that are about 100 nucleotides to about 300 nucleotides in length.

3. The method of claim 1, wherein about 100 ng of isolated DNA is incubated with the pA-Tn5.

4. The method of claim 1, wherein the pA-Tn5 attaches a first tag to the 5′ end of the DNA fragments.

5. The method of claim 4, further comprising ligating a second tag to the 3′ end of the DNA fragments through oligo-replacement.

6. The method of claim 1, wherein the isolated DNA is from a cell.

7. The method of claim 1, wherein the method does not use an antibody or other DNA-targeting molecule to target the pA-Tn5 to the DNA.

8. The method of claim 1, further comprising amplifying one or more of the DNA fragments from the DNA library with polymerase chain reaction (PCR).

9. The method of claim 8, further comprising sequencing the amplified DNA fragments.

10. A method of identifying a modification of interest in isolated DNA, the method comprising:
incubating the isolated DNA with pA-Tn5 to thereby generate DNA fragments;
isolating DNA fragments having the modification of interest; and
identifying the modification of interest from the DNA fragments.

11. The method of claim 10, wherein the isolating comprises immunoprecipitating the DNA fragments having the modification of interest with an antibody.

12. The method of claim 11, wherein the antibody is a methylation-specific antibody.

13. The method of claim 12, wherein the methylation-specific antibody is an anti-5mC antibody.

14. The method of claim 10, wherein the identifying comprises amplifying immunoprecipitated DNA fragments with polymerase chain reaction (PCR).

15. The method of claim 10, wherein the pA-Tn5 attaches a first tag to the 5′ end of the DNA fragments.

16. The method of claim 15, further comprising ligating a second tag to the 3′ end of the DNA fragments through oligo-replacement.

17. The method of claim 11, further comprising denaturing the DNA fragments into single stranded DNA fragments prior to the immunoprecipitating.

18. The method of claim 10, wherein the DNA fragments are about 100 nucleotides to about 300 nucleotides in length.

19. A method of diagnosing cancer in an individual, the method comprising:
incubating DNA obtained from the individual with pA-Tn5 to thereby generate DNA fragments;
isolating DNA fragments having a modification of interest; and
identifying the position of the modification of interest from the DNA fragments, thereby diagnosing cancer.

20. The method of claim 19, wherein the isolating comprises immunoprecipitating the DNA fragments having the modification of interest with an antibody.
